(19) United States
(12) Patent Application Publication
(10) Pub. No.: US 2010/0209413 A1
(43) Pub. Date: Aug. 19, 2010

(54) SERPINE1 POLYMORPHISMS ARE PREDICTIVE OF RESPONSE TO ACTIVATED PROTEIN C ADMINISTRATION AND RISK OF DEATH

(76) Inventors: Keith R. Walley, North Vancouver (CA); James R. Russell, Vancouver (CA); Asim Sarosh Siddiqui, San Francisco, CA (US); Anthony Gordon, London (GB); Mark D. Williams, Franklin, IN (US); William Louis Macias, Indianapolis, IN (US); Sandra Close Kirkwood, Fortville, IN (US)

Correspondence Address: BROWDY AND NEIMARK, P.L.L.C.;624 NINTH STREET, NW, SUITE 300, WASHINGTON, DC, 20001-5303, US

(21) Appl. No.: 12/295,232

(22) PCT Filed: Feb. 18, 2008
(86) PCT No.: PCT/CA08/00305

§ 371 Date: Mar. 22, 2010

Related U.S. Application Data
(60) Provisional application No. 60/901,672, filed on Feb. 16, 2007.
(60) Provisional application No. 60/907,188, filed on Mar. 23, 2007.

(51) Int. Cl. A61K 38/48; A61P 29/00; C12Q 1/68; C07H 21/00; C07K 2/00; C40B 40/06; C40B 40/10

(52) U.S. Cl. 424/94.64; 435/6; 536/23.1; 530/300; 506/16; 506/18

(57) ABSTRACT

Methods, oligonucleotides arrays etc. for treating inflammatory conditions and of predicting subject outcome based on polymorphisms in SERPINE1 and/or PROC, alone or in combination, wherein the method of treatment includes administering to the subject an anti-inflammatory agent or an anti-coagulant agent, wherein said subject is determined to have an improved response genotype or combination.

### BACKGROUND

Recent studies have demonstrated a relationship between genotype and response to pharmacological therapeutics (i.e. pharmacogenomics). Genentech's HERCEPTIN® was not effective in its overall Phase III trial but was shown to be of therapeutic benefit in a genetic subset of patients with human epidermal growth factor receptor 2 (HER2)-positive metastatic breast cancer. Similarly, Novartis' GLEEVEC® is only indicated for the subset of chronic myeloid leukemia patients who carry a reciprocal translocation between chromosomes 9 and 22 (i.e. the Philadelphia chromosome).

The septic inflammatory response involves complex cross-talk within and between the inflammation, coagulation and apoptosis pathways. Homeostatic imbalance of these and other counter-regulatory pathways can lead to altered clinical outcome in subjects with inflammatory conditions such as severe sepsis. Naturally-occurring genetic variation in human populations is one mechanism that can induce such a response. Furthermore, the genotype of an individual has been demonstrated to predict clinical outcome with respect to various inflammatory and infectious phenotypes (ARCAROLI J et al. Shock (2005) 24(4):300-12; SUTHERLAND A M et al. Crit Care Med (2005) 33(3):638-44; WATANABE E et al. J Trauma (2005) 59(5):1181-9; GORDON A C et al. Shock (2006) 25(1):88-93).

Serpin Peptidase Inhibitor, Clade E, member 1 (SERPINE1) gene is approximately 11.9 kb in length and located at chromosome 7q21-22 (http://genome.ucsc.edu). In its protein form, SERPINE1 is known as Human Plasminogen Activator Inhibitor protein (PAI-1), is 402 amino acids in length and is expressed primarily in liver, smooth muscle cells, adipocytes and platelets; it is also secreted into the plasma (BINDER B R et al. News Physiol Sci (2002) 17:56-61). Two SERPINE1 mRNA transcripts have been described that vary by approximately 1 kb in the length of their 3′ UTR (FATTAL P G and BILLADELLO J J Nucleic Acids Res (1993) 21(6):1463-1466). The reference gene sequence for Homo Sapiens SERPINE1 is annotated in GenBank under accession number NM_{—}000602.1 (GI:10835158). PAI-1 and SERPINE1 are used interchangeably throughout this application to refer to both the gene and its protein product.

The differential expression of SERPINE1 protein is observed to be correlated with a wide spectrum of inflammatory phenotypes including systemic inflammatory response syndrome (SIRS; GARCIA-FERNANDEZ N et al. Nephron (2002) 92(1):97-104), sepsis or septic shock (HERMANS P W et al. Lancet (1999) 354(9178):556-60; WESTENDORP R G J et al. Lancet (1999) 354:561-563), cardiovascular disease (FUJITA H et al. Circ Res (2006) 98(5):626-34; ZAK I et al. Clin Chem Acta (2005)362(1-2):110-18), ischemic stroke (SMITH A et al. Circulation 2005 112(20)3080-7), type 2 diabetes (MEIGS J B et al. Obesity (2006) 14(5):753-8), metabolic syndrome (PALOMO I et al. In J Mol Med (2006):18(5):969-74) as well as other inflammatory conditions induced by cytokine expression (DONG J et al. Arterioscler Thromb Vasc Biol (2005) 25(5):1078-84). Increased SERPINE1 levels are also associated with poor outcome in metastatic breast cancer (LEISSNER P et al. BMC Cancer 2006 31(6):216).

DAWSON et al. (J Biol Chem (1993) 268(15):10739-45) identified a 1 base pair (bp) insertion/deletion polymorphism at position −675 of the SERPINE1 promoter sequence which corresponds to position 837 of NM_{—}000602.1 (GI:10835158). This polymorphism is commonly referred to as 4G/5G and is associated with increased SERPINE1 levels (DAWSON S J et al. (1993); DAWSON S J et al. Arterioscler Thromb (1991) 11(1):183-90). The 4G allele of this single nucleotide polymorphism (SNP) is associated with increased risk of deep venous thrombosis (SEGUI R et al. Br J Haem (2000) 111(1):183-190), stroke (HINDORFF L et al. J Cardiovascular Risk (2002) 9(2):131-7), acute myocardial infarction (BOEKHOLDT S M et al. Circulation (2001) 104(25):3063-8; ERIKSSON P et al. PNAS (1995) 92(6):1851-5), late lumen loss after coronary artery stent placement (ORTLEPPG J R et al. Clin Cardiol (2001) 24(9):585-91) and sudden cardiac death (ANVARI A et al. Thrombosis Research (2001) 103(2):103-7; MIKKELSSON J et al. Thromb Haemost (2000) 84(1):78-82). In critically ill subjects, the 4G allele is also associated with decreased survival in cases of severe trauma (MENGES T et al. Lancet (2001) 357(9262):1096-7) and in cases of sepsis and septic shock caused by Neisseria meningitidis (HERMANS P W et al. Lancet (1999) 354(9178):556-60; WESTENDORP R G J et al. Lancet (1999) 354:561-563). The PAI-1 4G/4G genotype has also been associated with adverse patient outcomes (MENGES T et al. (2001); HERMANS P W et al. (1999); WESTENDORP R G J et al. (1999) ENDLER G et al. Br J Haem (2000) 110(2):469-71; GARDEMANN A et al. Thromb Haemost (1999) 82(3):1121-6; HOOPER W C et al. Thromb Res (2000) 99(3):223-30, JONES K et al. Eur J Vasc Endovasc Surg (2002) 23(5):421-5; HARALAMBOUS E et al. Crit Care Med (2003) 31(12):2788-93; and ROEST M et al. Circulation (2000) 101(1):67-70). The 4G/4G genotype of SERPINE1 is also associated with increased SERPINE1 levels in patients with acute lung injury (RUSSELL JA Crit Care Med (2003) 31(4):S243-S247).

The human Protein C gene (PROC) maps to chromosome 2q13-q14. The reference Homo sapiens PROC gene sequence is listed in GenBank under accession number NM 000312 (GI:109389366). PROC encodes a precursor protein consisting of 461 amino acids. Protein C is synthesized primarily in the liver and secreted into the plasma where it exists in its inactive form until it is cleaved by the thrombin: thrombomodulin complex. Activated Protein C (APC) modulates the coagulation cascade by inactivating coagulation factor Va (WALKER F J. et al. Biochim Biophys Acta (1979) 571(2):333-42) and coagulation factor VIIIa (FULCHER C A. et al. Blood (1984) 63(2):486-9). APC also attenuates the synthesis of plasminogen activator inhibitor type 1 (SERPINE1) (VAN HINSBERGH V W. et al. Blood (1985) 65(2):444-51).

APC demonstrates anti-inflammatory activity through binding to the Protein C Receptor (PROCR) to activate the Factor 2 Receptor (F2R or PAR1; RIEWALD M. et al. Science (2002) 296(5574):1880-2). F2R is a G protein-coupled receptor whose activation decreases downstream NFκB signaling and subsequent TNFα, IL1β, and IL6 expression (GREY S T. et al. Journal of Immunology (1994) 153(8):3664-72; HANCOCK W W. et al. Transplantation (1995) 60(12):1525-32; and MURAKAMI K. et al. American Journal of Physiology (1997) 272(2 Pt 1):L197-202). APC also decreases neutrophil adhesion to endothelial cells, decreases neutrophil chemotaxis and decreases apoptosis of endothelial cells and neurons (GRINNELL B W. et al. Glycobiology (1994) 4(2):221-5; JOYCE D E. et al. J Biol Chem (2001) 276(14):11199-203; STURN D H. et al. Blood (2003) 102(4):1499-505; and LIU D. et al. Nat Med (2004) 10(12):1379-83). Accordingly, APC has been implicated as having a central role in the pathophysiology of the systemic inflammatory response syndrome and the inflammatory sequelae arising from sepsis.

Decreased plasma levels of protein C are observed in association with the inflammatory response arising from sepsis, major surgery, or shock (GRIFFIN J H. et al. Blood (1982) 60(1):261-4; BLAMEY S L. et al. Thromb Haemost (1985) 54(3):622-5; TAYLOR F B. et al. Journal of Clinical Investigation (1987) 79(3):918-25; HESSELVIK J F. et al. Thromb Haemost (1991) 65(2):126-9; FUNVANDRAAT K. et al. Thromb Haemost (1995) 73(1):15-20; and FAUST S N. et al. N Engl J Med (2001) 345(6):408-16) and is related to poor outcome (LORENTE J A. et al. Chest (1993) 103(5):1536-42; VERVLOET M G. et al. Semin Thromb Hemost (1998) 24(1):33-44; FISHER C J. Jr. and YAN S B. Crit Care Med (2000) 28(9 Suppl):S49-56; YAN S B. and DHAINAUT J F. Crit Care Med (2001) 29(7 Suppl):S69-74; and LAY A J. et al. Blood (2006; Epub ahead of print). The expression of endothelial cell proteins such as thrombomodulin and protein C receptor (PROCR) is also impaired by pro-inflammatory cytokines and thus may also serve as a mechanism by which Protein C function is abrogated (STEARNS-KUROSAWA D J. et al. Proceedings of the National Academy of Sciences of the United States of America (1996) 93(19):10212-6).

Therapeutic agents for severe sepsis often target one or more of the pathways intrinsic to inflammation and infection. In particular, XIGRIS™ (drotrecogin alfa (activated), activated protein C, APC) having anti-inflammatory, anti-coagulant, pro-fibrinolytic and anti-apoptotic activity, has been observed to decrease 28-day mortality in both experimental sepsis models (LAY A J et al. Blood (2006; Epub ahead of print) and in the Phase III PROWESS severe sepsis trial (BERNARD G R. et al. New England Journal of Medicine (2001) 344(10):699-709; MACIAS W L et al. Crit Care (2005) 9(Suppl4):S38-45).

Several international applications disclose PROC and/or SERPINE 1 polymorphisms in association with inflammatory conditions. For example, WO05087789; WO03100090; and WO04083457.

### SUMMARY

This invention is based in part on the surprising discovery that certain single nucleotide polymorphisms (SNPs) from the SERPINE1 and PROC genes are predictive or indicative of the responsiveness or non-responsiveness of a subject having an inflammatory condition to treatment of the inflammatory condition with an anti-inflammatory agent or an anti-coagulant agent, based upon the subject having a particular SERPINE1 and PROC genotype described herein.

This invention is based, in part, on the identification of a particular nucleotide (allele) or genotype at the site of a given SNP or combination(s) of SNPs that may be associated with an increased likelihood of responsiveness or non-responsiveness to treatment of an inflammatory condition with an anti-inflammatory agent or an anti-coagulant agent in a subject having an inflammatory condition. Genotypes that are associated with responsiveness to an anti-inflammatory agent or an anti-coagulant agent are referred to herein as “improved response genotype(s)” (IRG; for a genotype at a single SNP), or “improved response genotype combination(s)” (IRGC; for genotypes at a combination(s) of SNPs). Alternatively, genotypes that are associated with non-responsiveness to an anti-inflammatory agent or an anti-coagulant agent are referred to herein as a “non-response genotype(s)” (NRG; for a genotype at a single SNP) or “non-response genotype combination(s)” (NRGC; for genotypes at a combination(s) of SNPs). As illustrated herein, subjects having an IRG or IRGC are more likely to have an improved response to, and benefit from, an anti-inflammatory agent or an anti-coagulant agent. Subjects having a NRG or NRGC are less likely to respond to, or benefit from, the same anti-inflammatory agent or anti-coagulant agent.

This invention is also based, in part, on the surprising discovery that SNPs from SERPINE1 and PROC alone or in combination(s) are useful in predicting whether or not a subject is more or less likely to have a serious adverse event from the administration of an anti-inflammatory agent or an anti-coagulant agent. Furthermore, the invention is based, in part, on the surprising result that the subjects who are generally less likely to have a serious adverse event following the administration of an anti-inflammatory agent or an anti-coagulant agent are subjects having an IRG or IRGC, and that the subjects who are generally more likely to have a serious adverse event following the administration of an anti-inflammatory agent or an anti-coagulant agent are subjects having an NRG or NRGC. Furthermore, there are provided herein, SNPs in linkage disequilibrium (LD) to SERPINE1 and PROC SNPs, also useful in predicting the response a subject with an inflammatory condition will have to treatment with an anti-inflammatory agent or an anti-coagulant agent.

This invention also is based in part on the discovery that certain genotypes at SNPs in SERPINE1 and PROC, alone or in combination(s), are predictive or indicative of subject outcome, wherein subject outcome is the ability of the subject to recover from an inflammatory condition in the absence of treatment with an anti-inflammatory agent or anti-coagulant agent, based on having a particular SERPINE1 or PROC genotype described herein as compared to a subject not having that genotype. In general, IRG and IRGC genotypes are associated with a reduced likelihood of recovery, and NRG and NRGC genotypes are associated with an increased likelihood of recovery, in the absence of treatment with an anti-inflammatory agent or an anti-coagulant agent.

In some embodiments of the invention, the SERPINE1 SNP is selected from rs7242; rs2070682; rs11178; rs2227706; and rs2227684; or a polymorphic marker in linkage disequilibrium thereto. In some embodiments, the PROC SNP is rs2069912; or a polymorphic marker in linkage disequilibrium thereto.

The invention also provides for “mixed response genotype combination(s)” (MRGC), wherein for a combination(s) of two SNPs, there is a response allele at one polymorphism site, but not at the other. In general, MRGC are associated with outcomes that are intermediate between IRGC and NRCG.

By way of illustration, and not limiting the generality of the forgoing, in one embodiment of the invention, a genotype combination(s) of two SNPs, rs2069912 in PROC and rs7242 in SERPINE1, is used to predict various subject outcomes. The responsive alleles are T for rs7242 and C for rs2069912, as shown in the Examples. The classification of genotypes in these SNPs into improved response genotype combinations (IRGC), mixed response genotype combinations (MRGC) and non-response genotype combination(s) (NRGC) is summarized below,
IRGC for (rs7242/rs2069912): TT/CC; GT/CC; TT/CT; and GT/CT.
MRGC for (rs7242/rs2069912): GG/CC; GG/CT; TT/TT; and GT/TT.
NRGC for (rs7242/rs2069912): GG/TT.

For example, a subject having an IRGC genotype would have at least one responsive allele in each of the genes. For example, a subject having a MRGC genotype would have at least one responsive allele in one gene but not the other. For example, a subject having a NRGC genotype would not have any responsive allele in either gene.

Furthermore, various SNPs from SERPINE1 and PROC and SNPs in linkage disequilibrium (LD) thereto are provided which are useful for subject screening, as an indication of subject outcome, or for prognosis for recovery from an inflammatory condition. There are also provided herein SNPs from SERPINE1 and PROC and SNPs in linkage disequilibrium (LD) thereto, which are also useful in predicting the response a subject's with an inflammatory condition will have to treatment with an anti-inflammatory agent or an anti-coagulant agent.

The method may further include selective administration of an anti-inflammatory agent or an anti-coagulant agent; wherein a subject has one or more improved response genotypes(s), improved response genotype combinations, or mixed response genotype combinations. The method may further include selectively not administering the anti-inflammatory agent or the anti-coagulant agent; wherein a subject does not have one or more improved response genotype(s) or improved response genotype combinations, or mixed response genotype combinations.

In accordance with one aspect of the invention, methods are provided for identifying a subject having one or more improved response genotype(s), the method including determining a genotype of the subject at one or more polymorphic sites, wherein the genotype may be indicative of the subject's response to an anti-inflammatory agent or an anti-coagulant agent, wherein the polymorphic site(s) are selected from one or more of the following: rs7242; rs2070682; rs11178; rs2227706; and rs2227684; or one or more polymorphic sites in linkage disequilibrium thereto. The method may further include determining a genotype of said subject at rs2069912 or one or more polymorphic sites in linkage disequilibrium thereto. The method may further include obtaining polymorphism sequence information for the subject. The genotype may be determined using a nucleic acid sample from the subject. The method may further include obtaining the nucleic acid sample from the subject. The method may further include selective administration of an anti-inflammatory agent or an anti-coagulant agent; wherein a subject has one or more improved response genotype(s) or improved response genotype combinations or mixed response genotype combinations. The method may further include selectively not administering the anti-inflammatory agent or the anti-coagulant agent; wherein a subject does not have one or more improved response genotype(s) or improved response genotype combinations. The method may further include selectively not administering the anti-inflammatory agent or the anti-coagulant agent, wherein a subject has one or more non-response genotype(s) or non-response genotype combination(s). The method may further include selective administration of an anti-inflammatory agent or an anti-coagulant agent; wherein a subject has one or more mixed response genotype combination(s).

In accordance with further aspects of the invention, methods are provided for identifying a subject having one or more reduced serious adverse event genotype(s) or one or more serious adverse event genotype combination(s), the method including determining a genotype of said subject at one or more polymorphic sites, wherein said genotype is respectively indicative of the subject's reduced likelihood of or increased likelihood of having a serious adverse event in response to the administration of an anti-inflammatory agent or an anti-coagulant agent, wherein the polymorphic site(s) are selected from one or more of the following: rs2069912; rs7242; rs2070682; rs11178; rs2227706; and rs2227684; one or more polymorphic sites in linkage disequilibrium thereto; and a combination(s) thereof. The method may further include determining a genotype of said subject at rs2069912 or one or more polymorphic sites in linkage disequilibrium thereto. The method may further include obtaining polymorphism sequence information for the subject. The genotype may be determined using a nucleic acid sample from the subject. The method may further include obtaining the nucleic acid sample from the subject. The method may further include selectively not administering the anti-inflammatory agent or the anti-coagulant agent; wherein a subject has one or more serious adverse event or serious adverse event genotype combination(s). The method may further include selective administration of an anti-inflammatory agent or an anti-coagulant agent; wherein a subject has one or more mixed response genotype combination(s). In some embodiments, the serious adverse events may be bleeding, non-bleeding or thrombotic in nature. The serious adverse event genotype(s) may be selected from one or more of the following: rs2069912 TT; rs7242 GG; rs2070682 CC; rs11178 CC; rs2227706 AA; rs2227684AA; one or more polymorphic sites in linkage disequilibrium thereto; and a combination thereof. The serious adverse event genotype combination(s) may be selected from one or more of the following: rs7242 GG/rs2069912 TT; rs2070682 CC/rs2069912 TT; rs11178 CC/rs2069912 TT; rs2227706 AA/rs2069912 TT; rs2227684 AA/rs2069912 TT; and one or more polymorphic sites in linkage disequilibrium thereto.

In accordance with further aspects of the invention, methods are provided for selecting a group of subjects for determining the efficacy of a candidate drug known or suspected of being useful for the treatment of an inflammatory condition; the method including determining a genotype at one or more of the following polymorphic sites: rs7242; rs2070682; rs11178; rs2227706; and rs2227684; or one or more polymorphic sites in linkage disequilibrium thereto; wherein the genotype is indicative of the subject's response to the candidate drug and sorting subjects based on their genotype. The method may further include; administering the candidate drug to the subjects or a subset of subjects and determining each subject's ability to recover from the inflammatory condition. The method may further include comparing subject response to the candidate drug based on genotype of the subject.

In accordance with further aspects of the invention, methods are provided for treating an inflammatory condition in a subject in need thereof; the method including administering to the subject an anti-inflammatory agent or an anti-coagulant agent; wherein the subject is determined to have an improved response genotype in one or more of the following sites: rs7242; rs2070682; rs11178; rs2227706; and rs2227684; or one or more polymorphic sites in linkage disequilibrium thereto.

In accordance with further aspects of the invention, methods are provided for selecting a subject for the treatment of an inflammatory condition with an anti-inflammatory agent or an anti-coagulant agent; including the step of identifying a subject having an improved response genotype in one or more of the following sites: rs7242; rs2070682; rs11178; rs2227706; and rs2227684; or one or more polymorphic sites in linkage disequilibrium thereto; wherein the identification of a subject with the improved response genotype is predictive of increased responsiveness to the treatment of the inflammatory condition with the anti-inflammatory agent or the anti-coagulant agent.

In accordance with further aspects of the invention, methods are provided for obtaining a prognosis for a subject having, or at risk of developing, an inflammatory condition, the method including determining a genotype of said subject which includes one or more polymorphic sites in the subject's SERPINE1 and PROC sequences or a combination(s) thereof, wherein said genotype is indicative of an ability of the subject to recover from the inflammatory condition. The method may further involve determination of the genotype for one or more polymorphic sites in SERPINE1 and PROC sequences for the subject. The genotypes of the SERPINE1 and PROC sequences may be taken alone or in combination(s).

The method may further include determining a genotype of said subject at rs2069912 or one or more polymorphic sites in linkage disequilibrium thereto.

In accordance with another aspect of the invention, there is provided a use of an anti-inflammatory agent or an anti-coagulant agent in the manufacture of a medicament for the treatment of an inflammatory condition; wherein the subjects treated are determined to have an improved response genotype selected from one or more of the following: rs7242; rs2070682; rs11178; rs2227706; and rs2227684; or one or more polymorphic sites in linkage disequilibrium thereto.

In accordance with another aspect of the invention, there is provided a use of an anti-inflammatory agent or an anti-coagulant agent in the manufacture of a medicament for the treatment of an inflammatory condition in a subset of subjects; wherein the subset of subjects are determined to have an improved response genotype at one or more of: rs7242; rs2070682; rs11178; rs2227706; and rs2227684; or one or more polymorphic sites in linkage disequilibrium thereto.

The use may further include determining a genotype of said subject at rs2069912 or one or more polymorphic sites in linkage disequilibrium thereto.

In accordance with another aspect of the invention, there is provided a method of treating an inflammatory condition in a subject in need thereof, the method including administering to the subject an anti-inflammatory agent or an anti-coagulant agent, wherein said subject is determined to have a reduced serious adverse event genotype in one or more of the following sites: rs2069912; rs7242; rs2070682; rs11178; rs2227706; and rs2227684; one or more polymorphic sites in linkage disequilibrium thereto; and combinations thereof.

In accordance with another aspect of the invention, there is provided a method of selecting a subject for the treatment of an inflammatory condition with an anti-inflammatory agent or an anti-coagulant agent, including the step of identifying a subject having a reduced serious adverse event genotype in one or more of the following sites: rs2069912; rs7242; rs2070682; rs11178; rs2227706; and rs2227684; one or more polymorphic sites in linkage disequilibrium thereto; and combinations thereof, wherein the identification of a subject with the reduced serious adverse event genotype is predictive of increased responsiveness to the treatment of the inflammatory condition with the anti-inflammatory agent or the anti-coagulant agent.

In accordance with another aspect of the invention, there is provided a use of an anti-inflammatory agent or an anti-coagulant agent in the manufacture of a medicament for the treatment of an inflammatory condition, wherein the subjects treated are determined to have a reduced serious adverse event genotype selected from one or more of the following: rs2069912; rs7242; rs2070682; rs11178; rs2227706; and rs2227684; one or more polymorphic sites in linkage disequilibrium thereto; and combinations thereof.

In accordance with another aspect of the invention, there is provided a use of an anti-inflammatory agent or an anti-coagulant agent in the manufacture of a medicament for the treatment of an inflammatory condition in a subset of subjects, wherein the subset of subjects have an reduced serious adverse event genotype at one or more of: rs2069912; rs7242; rs2070682; rs11178; rs2227706; and rs2227684; one or more polymorphic sites in linkage disequilibrium thereto; and combinations thereof.

The method or use may further include determining the subject's APACHE II score as an assessment of subject risk. The method or use may further or alternatively include determining the number of organ system failures for the subject as an assessment of subject risk. The method or use may further or alternatively include determining the type of organ system failures for the subject as an assessment of subject risk. The subject's APACHE II score may be indicative of an increased risk when ≧25. 2 or more organ system failures may be indicative of increased subject risk. The type of organ system failures may be indicative of increased subject risk. Alternatively, the genotype determination may be used to select who to treat (for example based on IRG, NRG, IRGC, MRGC or NRGC) and protein C level or SERPINE1 or PROC/SERPINE1 ratio may be used to decide the dose and/or duration of treatment with an anti-inflammatory agent or an anti-coagulant agent.

In accordance with further aspects of the invention, a commercial package is provided containing; as active pharmaceutical ingredient, a protein C or protein C like compound, together with instructions for its use for the curative or prophylactic treatment of an inflammatory condition in a subject; wherein the subject treated is determined to have an improved response genotype selected from the following: rs7242; rs2070682; rs11178; rs2227706; and rs2227684; or one or more polymorphic sites in linkage disequilibrium thereto. The subject treated may also have an improved response genotype at rs2069912 or one or more polymorphic sites in linkage disequilibrium thereto. The subject may also have one or more improved response genotypes, improved response genotype combinations, mixed response genotype combinations, or adverse event genotypes, as set out herein.

In accordance with further aspects of the invention, a method is provided for identifying a subject having one or more risk genotype(s); the method including determining a genotype of said subject at one or more polymorphic sites; wherein said genotype is indicative of the subject's ability to recover from an inflammatory condition; wherein the polymorphic site(s) are selected from one or more of the following: rs7242; rs2070682; rs11178; rs2227706; and rs2227684; or one or more polymorphic sites in linkage disequilibrium thereto. The method may further include determining a genotype of said subject at rs2069912 or one or more polymorphic sites in linkage disequilibrium thereto.

In accordance with another aspect of the invention, there is provided a kit for determining a genotype at a defined nucleotide position within a polymorphic site in a protein C or SERPINE1 sequence in a subject to predict a subject's response to an anti-inflammatory agent or an anti-coagulant agent, the kit including: a restriction enzyme capable of distinguishing alternate nucleotides at the polymorphic site; or a labeled oligonucleotide having sufficient complementary to the polymorphic site so as to be capable of hybridizing distinctively to said alternate. The kit may further include an oligonucleotide or a set of oligonucleotides operable to amplify a region including the polymorphic site. The kit may further include a polymerization agent. The kit may further include instructions for using the kit to determine genotype.

The anti-inflammatory agent or the anti-coagulant agent may be selected from any one or more of the following: activated protein C or protein C like compound; protein S or a protein S like drug; a factor Xa inhibitor such as tissue factor pathway inhibitor (TFPI) (e.g. TIFACOGIN™-alpha (Chiron) and the like) or a monoclonal antibody against tissue factor (TF); or a serine protease inhibitor (for example antithrombin III); platelet activating factor hydrolase; PAF-AH enzyme analogues; tissue plasminogen activator (tPA); heparin; thrombomodulin; or recombinant human thrombomodulins, including various derivatives and forms of thrombomodulin, such as soluble thrombomodulin (for example, SOLULIN™). The anti-inflammatory agent or the anti-coagulant agent may be activated protein C or protein C like compound. The activated protein C or protein C like compound may be drotrecogin alfa (activated).

In accordance with another aspect of the invention, methods are provided for treatment of an inflammatory condition in an eligible subject by administering a treatment option, such as a anti-inflammatory agent or the anti-coagulant agent, after first determining if a subject is an eligible subject on the basis of the genetic sequence information or genotype information disclosed herein. Where the method of treatment of an inflammatory condition in an eligible subject may comprise the following: a) determining if a subject is an eligible subject on the basis of the presence or absence of one or more polymorphic sites in the SERPINE1 sequence and may further include the presence or absence of polymorphisms in the PROC sequence wherein said genotype is indicative of the subject's ability to recover from an inflammatory condition b) administering anti-inflammatory agent or the anti-coagulant agent to the eligible subject. More specifically, the method of treatment of an inflammatory condition in an eligible subject may comprise: a) determining if a subject is an eligible subject on the basis of the presence or absence of one or more polymorphic sites; wherein said genotype is indicative of the subject's ability to recover from an inflammatory condition; wherein the polymorphic site(s) are selected from one or more of the following: rs7242; rs2070682; rs11178; rs2227706; and rs2227684; or one or more polymorphic sites in linkage disequilibrium thereto. The method may further include determining a genotype of said subject at rs2069912 or one or more polymorphic sites in linkage disequilibrium thereto and b) administering a anti-inflammatory agent or the anti-coagulant agent selected from among activated protein C (e.g. XIGRIS™-drotrecogin alfa-recombinant human activated protein C (Eli Lilly)), protein S or a protein S like drug; a factor Xa inhibitor such as tissue factor pathway inhibitor (TFPI) (e.g. TIFACOGIN™-alpha (Chiron) and the like) or a monoclonal antibody against tissue factor (TF); or a serine protease inhibitor (for example antithrombin III); platelet activating factor hydrolase; PAF-AH enzyme analogues; tissue plasminogen activator (tPA); heparin; thrombomodulin; or recombinant human thrombomodulins, including various derivatives and forms of thrombomodulin, such as soluble thrombomodulin (for example, SOLULIN™). Furthermore, the anti-inflammatory agent or the anti-coagulant agent may be activated protein C and/or a derivative thereof (including glycosylation mutants), alone or in combination(s) or in combination(s) with other therapeutic agents as described herein. An improved response to a therapeutic agent may include an improvement subsequent to administration of the therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR >1.5], renal and/or hepatic).

In accordance with another aspect of the invention, methods are provided for treating an inflammatory condition in a subject in need thereof, the method including administering to the subject a protein C or protein C like compound, wherein the subject is determined to have an improved response genotype in one or more of the following sites: rs7242; rs2070682; rs11178; rs2227706; and rs2227684; or one or more polymorphic sites in linkage disequilibrium thereto.

In accordance with another aspect of the invention, methods are provided for increasing likelihood of effectiveness of a protein C treatment or protein C like compound treatment, the method including administering an inflammatory condition treating dose of the protein C or protein C like compound to a subject, wherein the subject is determined to have an improved response genotype in one or more of the following sites: rs7242; rs2070682; rs11178; rs2227706; and rs2227684; or one or more polymorphic sites in linkage disequilibrium thereto.

The method may further include determining a genotype of the subject at rs2069912 or one or more polymorphic sites in linkage disequilibrium thereto. The inflammatory condition may be selected from: SIRS; severe sepsis; sepsis; and septic shock. The inflammatory condition may be severe sepsis. The protein C or protein C like compound may be drotrecogin alfa (activated). The subject's improved response genotype may be determined for rs7242 and rs2069912. The subject's improved response genotype may be selected from the following IRGs or IRGCs: rs7242 GT; rs7242 TT; rs2070682 CT; rs2070682 TT; rs11178 CT; rs11178 TT; rs2227706 AG; rs2227706 GG; rs2227684AG; rs2227684 GG; rs7242 GT/rs2069912 CC; rs7242 GT/rs2069912 CT; rs7242 TT/rs2069912 CT; and rs7242 TT/rs2069912 CC. The method may further include determining the subject's APACHE II score as an assessment of subject risk. The subject's APACHE II score may be indicative of an increased risk when ≧25.

In accordance with another aspect of the invention, methods are provided for selecting subjects for non-treatment of an inflammatory condition in a subject in need thereof, the method including selectively not administering to the subject a protein C or protein C like compound, wherein the subject is determined to have an non-response genotype in one or more of the following sites: rs7242; rs2070682; rs11178; rs2227706; and rs2227684; or one or more polymorphic sites in linkage disequilibrium thereto.

The method may further include determining a genotype of the subject at rs2069912 or one or more polymorphic sites in linkage disequilibrium thereto. The inflammatory condition may be selected from: SIRS; severe sepsis; sepsis; and septic shock. The inflammatory condition may be severe sepsis. The protein C or protein C like compound may be drotrecogin alfa (activated). The subject's non-response genotype may be determined for rs7242 and rs2069912. The subject's non-response genotype may be selected from the following NRGs or NRGCs: rs7242 GG; rs2070682 CC; rs11178 CC; rs2227706 AA; rs2227684AA; rs7242 GG/rs2069912 TT; rs2070682 CC/rs2069912 TT; rs11178 CC/rs2069912 TT; rs2227706 AA/rs2069912 TT; rs2227684 AA/rs2069912 TT. The method may further include determining the subject's APACHE II score as an assessment of subject risk. The subject's APACHE II score may be indicative of an increased risk when ≧25.

In accordance with another aspect of the invention, there is provided a use of a protein C or protein C like compound in the treatment of an inflammatory condition, the use including administering to the subject, wherein the subject is determined to have an improved response genotype in one or more of the following sites: rs7242; rs2070682; rs11178; rs2227706; and rs2227684; or one or more polymorphic sites in linkage disequilibrium thereto.

In accordance with another aspect of the invention, there is provided a use of a protein C or protein C like compound in the manufacture of a medicament for the treatment of an inflammatory condition, wherein the subjects treated are determined to have an improved response genotype in one or more of the following sites: rs7242; rs2070682; rs11178; rs2227706; and rs2227684; or one or more polymorphic sites in linkage disequilibrium thereto.

The method may further include determining a genotype of the subject at rs2069912 or one or more polymorphic sites in linkage disequilibrium thereto. The inflammatory condition may be selected from: SIRS; severe sepsis; sepsis; and septic shock. The inflammatory condition may be severe sepsis. The protein C or protein C like compound may be drotrecogin alfa (activated). The subject's improved response genotype may be determined for rs7242 and rs2069912. The subject's IRG(s) or IRGC(s) or MRGC(s) may be selected from the following: rs7242 GT; rs7242 TT; rs2070682 CT; rs2070682 TT; rs11178 CT; rs11178 TT; rs2227706 AG; rs2227706 GG; rs2227684AG; rs2227684 GG; rs7242 GT/rs2069912 CC; rs7242 GT/rs2069912 CT; rs7242 GT/rs2069912 TT; rs7242 GG/rs2069912 CC; rs7242 TT/rs2069912 TT; rs7242 GG/rs2069912 CT; rs7242 TT/rs2069912 CT; rs7242 TT/rs2069912 CC; rs2070682 CT/rs2069912 CC; rs2070682 CT/rs2069912 CT; rs2070682 CT/rs2069912 TT; rs2070682 CC/rs2069912 CC; rs2070682 CC/rs2069912 CT; rs2070682 TT/rs2069912 TT; rs2070682 TT/rs2069912 CT; rs2070682 TT/rs2069912 CC; rs11178 CT/rs2069912 CC; rs11178 CT/rs2069912 CT; rs11178 CT/rs2069912 TT; rs11178 CC/rs2069912 CC; rs11178 CC/rs2069912 CT; rs11178 TT/rs2069912 TT; rs11178 TT/rs2069912 CT; rs11178 TT/rs2069912 CC; rs2227706 AG/rs2069912 CC; rs2227706 AG/rs2069912 CT; rs2227706 AG/rs2069912 TT; rs2227706 AA/rs2069912 CC; rs2227706 AA/rs2069912 CT; rs2227706 GG/rs2069912 TT; rs2227706 GG/rs2069912 CT; rs2227706 GG/rs2069912 CC; rs2227684 AG/rs2069912 CC; rs2227684 AG/rs2069912 CT; rs2227684 AG/rs2069912 TT; rs2227684 AA/rs2069912 CC; rs2227684 GG/rs2069912 TT; rs2227684 AA/rs2069912 CT; rs2227684 GG/rs2069912 CT; and rs2227684 GG/rs2069912 CC; or one or more polymorphic sites in linkage disequilibrium thereto. The method may further include determining the subject's APACHE II score as an assessment of subject risk. The subject's APACHE II score may be indicative of an increased risk when >25.

In accordance with another aspect of the invention, methods are provided for treatment of an inflammatory condition in an eligible subject comprising administering an anti-inflammatory agent or an anti-coagulant agent to an eligible subject. The eligible subject may be a subject having one or more polymorphic sites; wherein said genotype is indicative of the subject's ability to recover from an inflammatory condition; wherein the polymorphic site(s) are selected from one or more of the following: rs7242; rs2070682; rs11178; rs2227706; and rs2227684; or one or more polymorphic sites in linkage disequilibrium thereto. The method may further include a) determining a genotype of said subject at rs2069912 or one or more polymorphic sites in linkage disequilibrium thereto; b) administering anti-inflammatory agent or the anti-coagulant agent selected from among activated protein C (e.g. XIGRIS™-drotrecogin alfa-recombinant human activated protein C (Eli Lilly)), protein S or a protein S like drug; a factor Xa inhibitor such as tissue factor pathway inhibitor (TFPI) (e.g. TIFACOGIN™-alpha (Chiron) and the like) or a monoclonal antibody against tissue factor (TF); or a serine protease inhibitor (for example antithrombin III); platelet activating factor hydrolase; PAF-AH enzyme analogues; tissue plasminogen activator (tPA); heparin; or thrombomodulin; or recombinant human thrombomodulins, including various derivatives and forms of thrombomodulin, such as soluble thrombomodulin (for example SOLULIN™). Those skilled in the art are familiar with the dosage and administration of these and other treatment options. To determine a subject's eligibility, the presence or absence of polymorphisms in the SERPINE1 sequence and may further include the presence or absence of polymorphisms in the PROC sequence, may be determined as described herein.

Activated protein C (e.g. XIGRIS™ drotrecogin alfa-recombinant human activated protein C (Eli Lilly)), protein S or a protein S like drug; a factor Xa inhibitor such as tissue factor pathway inhibitor (TFPI) (e.g. TIFACOGIN™-alpha (Chiron) and the like) or a monoclonal antibody against tissue factor (TF); or a serine protease inhibitor (for example antithrombin III); platelet activating factor hydrolase; PAF-AH enzyme analogues; tissue plasminogen activator (tPA); heparin; thrombomodulin; recombinant human thrombomodulins (including various derivatives and forms of thrombomodulin, such as soluble thrombomodulin (for example SOLULIN™)) or other anti-inflammatory or anticoagulant therapeutic agents, may be useful in the manufacture of a medicament for the therapeutic treatment of an inflammatory condition in a subject having one or more of the polymorphisms in SERPINE1 and may further include the presence or absence of polymorphisms in the PROC sequence that are associated with decreased likelihood of recovery from an inflammatory condition. Furthermore these therapeutic agents may be useful in the preparation of an anti-sepsis agent in ready-to-use drug form for treating or preventing sepsis in a subject having one or more of the polymorphisms in SERPINE1 and may further include the presence or absence of polymorphisms in the PROC sequence that are associated with decreased likelihood of recovery from an inflammatory condition.

The improved response genotype(s) may be selected from one or more of the following: rs7242 GT; rs7242 TT; rs2070682 CT; rs2070682 TT; rs11178 CT; rs11178 TT; rs2227706 AG; rs2227706 GG; rs2227684AG; and rs2227684 GG; or one or more polymorphic sites in linkage disequilibrium thereto. The improved response genotype may alternatively be selected from one or more of the following combinations: rs7242 GT/rs2069912 CC; rs7242 GT/rs2069912 CT; rs7242 TT/rs2069912 CT; rs7242 TT/rs2069912 CC; rs2070682 CT/rs2069912 CC; rs2070682 CT/rs2069912 CT; rs2070682 TT/rs2069912 CT; rs2070682 TT/rs2069912 CC; rs11178 CT/rs2069912 CC; rs11178 CT/rs2069912 CT; rs11178 TT/rs2069912 CT; rs11178 TT/rs2069912 CC; rs2227706 AG/rs2069912 CC; rs2227706 AG/rs2069912 CT; rs2227706 GG/rs2069912 CT; rs2227706 GG/rs2069912 CC; rs2227684 AG/rs2069912 CC; rs2227684 AG/rs2069912 CT; rs2227684 GG/rs2069912 CT; and rs2227684 GG/rs2069912 CC; or one or more polymorphic sites in linkage disequilibrium thereto. The one or more polymorphic sites in linkage disequilibrium thereto may be selected from one or more of the polymorphic sites listed in TABLE 1B.

The reduced serious adverse event genotype(s) or combination(s) thereof may be selected from one or more of the following: rs2069912 CT; rs2069912 CC; rs7242 GT; rs7242 TT; rs2070682 CT; rs2070682 TT; rs11178 CT; rs11178 TT; rs2227706 AG; rs2227706 GG; rs2227684AG; rs2227684 GG; rs7242 GT/rs2069912 CC; rs7242 GT/rs2069912 CT; rs7242 GT/rs2069912 TT; rs7242 GG/rs2069912 CC; rs7242 TT/rs2069912 TT; rs7242 TT/rs2069912 CT; rs7242 TT/rs2069912 CC; rs7242 GG/rs2069912 CT; rs2070682 CT/rs2069912 CC; rs2070682 CT/rs2069912 CT; rs2070682 CT/rs2069912 TT; rs2070682 CC/rs2069912 CC; rs2070682 TT/rs2069912 TT; rs2070682 TT/rs2069912 CT; rs2070682 TT/rs2069912 CC; rs2070682 CC/rs2069912 CT; rs11178 CT/rs2069912 CC; rs11178 CT/rs2069912 CT; rs11178 CT/rs2069912 TT; rs11178 CC/rs2069912 CC; rs11178 TT/rs2069912 TT; rs11178 TT/rs2069912 CT; rs11178 TT/rs2069912 CC; rs11178 CC/rs2069912 CT; rs2227706 AG/rs2069912 CC; rs2227706 AG/rs2069912 CT; rs2227706 AG/rs2069912 TT; rs2227706 AA/rs2069912 CC; rs2227706 GG/rs2069912 TT; rs2227706 GG/rs2069912 CT; rs2227706 GG/rs2069912 CC; rs2227706 AA/rs2069912 CT; rs2227684 AG/rs2069912 CC; rs2227684 AG/rs2069912 CT; rs2227684 AG/rs2069912 TT; rs2227684 AA/rs2069912 CC; rs2227684 GG/rs2069912 TT; rs2227684 GG/rs2069912 CT; and rs2227684 GG/rs2069912 CC; rs2227684 AA/rs2069912 CT; or one or more polymorphic sites in linkage disequilibrium thereto.

The one or more polymorphic sites in linkage disequilibrium thereto may be selected from one or more of the polymorphic sites listed in TABLE 1B. The genotype may be determined using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data.

The subject may be critically ill with an inflammatory condition. The inflammatory condition may be selected from the group including: severe sepsis; sepsis; septicemia; pneumonia; septic shock; systemic inflammatory response syndrome (SIRS); Acute Respiratory Distress Syndrome (ARDS); acute lung injury; aspiration pneumonitis; infection; pancreatitis; bacteremia; peritonitis; abdominal abscess; inflammation due to trauma; inflammation due to surgery; chronic inflammatory disease; ischemia; ischemia-reperfusion injury of an organ or tissue; tissue damage due to disease; tissue damage due to chemotherapy or radiotherapy; and reactions to ingested; inhaled; infused; injected; or delivered substances; glomerulonephritis; bowel infection; opportunistic infections; and for subjects undergoing major surgery or dialysis; subjects who are immunocompromised; subjects on immunosuppressive agents; subjects with HIV/AIDS; subjects with suspected endocarditis; subjects with fever; subjects with fever of unknown origin; subjects with cystic fibrosis; subjects with diabetes mellitus; subjects with chronic renal failure; subjects with acute renal failure; oliguria; subjects with acute renal dysfunction; glomerulo-nephritis; interstitial-nephritis; acute tubular necrosis (ATN); subjects; subjects with bronchiectasis; subjects with chronic obstructive lung disease; chronic bronchitis; emphysema; or asthma; subjects with febrile neutropenia; subjects with meningitis; subjects with septic arthritis; subjects with urinary tract infection; subjects with necrotizing fasciitis; subjects with other suspected Group A streptococcus infection; subjects who have had a splenectomy; subjects with recurrent or suspected enterococcus infection; other medical and surgical conditions associated with increased risk of infection; Gram positive sepsis; Gram negative sepsis; culture negative sepsis; fungal sepsis; meningococcemia; post-pump syndrome; cardiac stun syndrome; myocardial infarction; stroke; congestive heart failure; hepatitis; epiglottitis; E. coli 0157:H7; malaria; gas gangrene; toxic shock syndrome; pre-eclampsia; eclampsia; HELLP syndrome; mycobacterial tuberculosis; Pneumocystis carinii pneumonia; Leishmaniasis; hemolytic uremic syndrome/thrombotic thrombocytopenic purpura; Dengue hemorrhagic fever; pelvic inflammatory disease; Legionella; Lyme disease; Influenza A; Epstein-Barr virus; encephalitis; inflammatory diseases and autoimmunity including Rheumatoid arthritis; osteoarthritis; progressive systemic sclerosis; systemic lupus erythematosus; inflammatory bowel disease; idiopathic pulmonary fibrosis; sarcoidosis; hypersensitivity pneumonitis; systemic vasculitis; Wegener's granulomatosis; transplants including heart; liver; lung kidney bone marrow; graft-versus-host disease; transplant rejection; sickle cell anemia; nephrotic syndrome; toxicity of agents such as OKT3; cytokine therapy; cirrhosis; disseminated intravascular coagulation (DIC); cardiogenic shock; and acute kidney injury. The inflammatory condition may be selected from: SIRS; severe sepsis; sepsis; and septic shock. The inflammatory condition may be severe sepsis.

The anti-inflammatory agent or the anti-coagulant agent may be a protein C or a protein C like compound. The protein C or protein C like compound may be drotrecogin alfa (activated).

In accordance with further aspects of the invention, two or more oligonucleotides or analogs thereof (for example locked nucleic acids) or peptide nucleic acids of about 10 to about 400 nucleotides are provided that hybridize specifically to a sequence contained in a human target sequence; a complementary sequence of the target sequence or RNA equivalent of the target sequence and wherein the oligonucleotides or peptide nucleic acids are operable in determining the presence or absence of two or more improved response genotype(s) in the target sequence selected from of the following polymorphic sites: rs7242; rs2070682; rs11178; rs2227706; rs2227684 and rs2069912 or one or more polymorphic sites in linkage disequilibrium thereto.

In accordance with further aspects of the invention, two or more oligonucleotides or peptide nucleic acids are provided which may be selected from the group consisting of: (a) an oligonucleotide or peptide nucleic acid that hybridizes under high stringency conditions to a nucleic acid molecule comprising SEQ ID NO:1 having a G at position 301 but not to a nucleic acid molecule comprising SEQ ID NO:1 having a T at position 301; (b) an oligonucleotide or peptide nucleic acid that hybridizes under high stringency conditions to a nucleic acid molecule comprising SEQ ID NO:1 having a T at position 301 but not to a nucleic acid molecule comprising SEQ ID NO:1 having a G at position 301; (c) an oligonucleotide or peptide nucleic acid that hybridizes under high stringency conditions to a nucleic acid molecule comprising SEQ ID NO:2 having a C at position 201 but not to a nucleic acid molecule comprising SEQ ID NO:2 having a T at position 201; (d) an oligonucleotide or peptide nucleic acid that hybridizes under high stringency conditions to a nucleic acid molecule comprising SEQ ID NO:2 having an T at position 201 but not to a nucleic acid molecule comprising SEQ ID NO:2 having a C at position 201; (e) an oligonucleotide or peptide nucleic acid that hybridizes under high stringency conditions to a nucleic acid molecule comprising SEQ ID NO:3 having a C at position 301 but not to a nucleic acid molecule comprising SEQ ID NO:3 having a T at position 301; (f) an oligonucleotide or peptide nucleic acid that hybridizes under high stringency conditions to a nucleic acid molecule comprising SEQ ID NO:3 having a T at position 301 but not to a nucleic acid molecule comprising SEQ ID NO:3 having a C at position 301; (g) an oligonucleotide or peptide nucleic acid that hybridizes under high stringency conditions to a nucleic acid molecule comprising SEQ ID NO:4 having a G at position 301 but not to a nucleic acid molecule comprising SEQ ID NO:4 having a C at position 301; (h) an oligonucleotide or peptide nucleic acid that hybridizes under high stringency conditions to a nucleic acid molecule comprising SEQ ID NO:4 having a C at position 301 but not to a nucleic acid molecule comprising SEQ ID NO:4 having a G at position 301; (i) an oligonucleotide or peptide nucleic acid that hybridizes under high stringency conditions to a nucleic acid molecule comprising SEQ ID NO:5 having a C at position 301 but not to a nucleic acid molecule comprising SEQ ID NO:5 having a T at position 301; (j) an oligonucleotide or peptide nucleic acid that hybridizes under high stringency conditions to a nucleic acid molecule comprising SEQ ID NO:5 having a T at position 301 but not to a nucleic acid molecule comprising SEQ ID NO:5 having a C at position 301; (k) an oligonucleotide or peptide nucleic acid that hybridizes under high stringency conditions to a nucleic acid molecule comprising SEQ ID NO:6 having a C at position 301 but not to a nucleic acid molecule comprising SEQ ID NO:6 having a T at position 301; (l) an oligonucleotide or peptide nucleic acid that hybridizes under high stringency conditions to a nucleic acid molecule comprising SEQ ID NO:6 having a T at position 301 but not to a nucleic acid molecule comprising SEQ ID NO:6 having a C at position 301; (m) an oligonucleotide or peptide nucleic acid that hybridizes under high stringency conditions to a nucleic acid molecule comprising SEQ ID NO:7 having a G at position 468 but not to a nucleic acid molecule comprising SEQ ID NO:7 having an A at position 468; (n) an oligonucleotide or peptide nucleic acid that hybridizes under high stringency conditions to a nucleic acid molecule comprising SEQ ID NO:7 having an A at position 468 but not to a nucleic acid molecule comprising SEQ ID NO:7 having a G at position 468; (o) an oligonucleotide or peptide nucleic acid that hybridizes under high stringency conditions to a nucleic acid molecule comprising SEQ ID NO:8 having a C at position 709 but not to a nucleic acid molecule comprising SEQ ID NO:8 having a T at position 709; (p) an oligonucleotide or peptide nucleic acid that hybridizes under high stringency conditions to a nucleic acid molecule comprising SEQ ID NO:8 having a T at position 709 but not to a nucleic acid molecule comprising SEQ ID NO:8 having a C at position 709; (q) an oligonucleotide or peptide nucleic acid that hybridizes under high stringency conditions to a nucleic acid molecule comprising SEQ ID NO:9 having a G at position 301 but not to a nucleic acid molecule comprising SEQ ID NO:9 having an A at position 301; (r) an oligonucleotide or peptide nucleic acid that hybridizes under high stringency conditions to a nucleic acid molecule comprising SEQ ID NO:9 having an A at position 301 but not to a nucleic acid molecule comprising SEQ ID NO:9 having a G at position 301; (s) an oligonucleotide or peptide nucleic acid that hybridizes under high stringency conditions to a nucleic acid molecule comprising SEQ ID NO:10 having an A at position 301 but not to a nucleic acid molecule comprising SEQ ID NO:10 having a G at position 301; (t) an oligonucleotide or peptide nucleic acid that hybridizes under high stringency conditions to a nucleic acid molecule comprising SEQ ID NO:10 having a G at position 301 but not to a nucleic acid molecule comprising SEQ ID NO:10 having an A at position 301; (u) an oligonucleotide or peptide nucleic acid that hybridizes under high stringency conditions to a nucleic acid molecule comprising SEQ ID NO:11 having a T at position 301 but not to a nucleic acid molecule comprising SEQ ID NO:11 having a C at position 301; (v) an oligonucleotide or peptide nucleic acid that hybridizes under high stringency conditions to a nucleic acid molecule comprising SEQ ID NO:11 having a C at position 301 but not to a nucleic acid molecule comprising SEQ ID NO:11 having a T at position 301; (w) an oligonucleotide or peptide nucleic acid that hybridizes under high stringency conditions to a nucleic acid molecule comprising SEQ ID NO:12 having a C at position 256 but not to a nucleic acid molecule comprising SEQ ID NO:12 having a T at position 256; (x) an oligonucleotide or peptide nucleic acid that hybridizes under high stringency conditions to a nucleic acid molecule comprising SEQ ID NO:12 having a T at position 256 but not to a nucleic acid molecule comprising SEQ ID NO:12 having a C at position 256; (y) an oligonucleotide or peptide nucleic acid that hybridizes under high stringency conditions to a nucleic acid molecule comprising SEQ ID NO:13 having a G at position 201 but not to a nucleic acid molecule comprising SEQ ID NO:13 having an A at position 201; (z) an oligonucleotide or peptide nucleic acid that hybridizes under high stringency conditions to a nucleic acid molecule comprising SEQ ID NO:13 having an A at position 201 but not to a nucleic acid molecule comprising SEQ ID NO:13 having a G at position 201; (aa) an oligonucleotide or peptide nucleic acid that hybridizes under high stringency conditions to a nucleic acid molecule comprising SEQ ID NO:14 having a G at position 201 but not to a nucleic acid molecule comprising SEQ ID NO:14 having a C at position 201; (bb) an oligonucleotide or peptide nucleic acid that hybridizes under high stringency conditions to a nucleic acid molecule comprising SEQ ID NO:14 having a C at position 201 but not to a nucleic acid molecule comprising SEQ ID NO:14 having a G at position 201; (cc) an oligonucleotide or peptide nucleic acid that hybridizes under high stringency conditions to a nucleic acid molecule comprising SEQ ID NO:15 having a C at position 501 but not to a nucleic acid molecule comprising SEQ ID NO:15 having a T at position 501; (dd) an oligonucleotide or peptide nucleic acid that hybridizes under high stringency conditions to a nucleic acid molecule comprising SEQ ID NO:15 having a T at position 501 but not to a nucleic acid molecule comprising SEQ ID NO:15 having a C at position 501; (ee) an oligonucleotide or peptide nucleic acid that hybridizes under high stringency conditions to a nucleic acid molecule comprising SEQ ID NO:16 having a C at position 201 but not to a nucleic acid molecule comprising SEQ ID NO:16 having a T at position 201; (ff) an oligonucleotide or peptide nucleic acid that hybridizes under high stringency conditions to a nucleic acid molecule comprising SEQ ID NO:16 having a T at position 201 but not to a nucleic acid molecule comprising SEQ ID NO:16 having a C at position 201; (gg) an oligonucleotide or peptide nucleic acid that hybridizes under high stringency conditions to a nucleic acid molecule comprising SEQ ID NO:17 having a G at position 301 but not to a nucleic acid molecule comprising SEQ ID NO:17 having an A at position 301; (hh) an oligonucleotide or peptide nucleic acid that hybridizes under high stringency conditions to a nucleic acid molecule comprising SEQ ID NO:17 having an A at position 301 but not to a nucleic acid molecule comprising SEQ ID NO:17 having a G at position 301; (ii) an oligonucleotide or peptide nucleic acid that hybridizes under high stringency conditions to a nucleic acid molecule comprising SEQ ID NO:18 having a G at position 980 but not to a nucleic acid molecule comprising SEQ ID NO:18 having a T at position 980; (jj) an oligonucleotide or peptide nucleic acid that hybridizes under high stringency conditions to a nucleic acid molecule comprising SEQ ID NO:18 having a T at position 980 but not to a nucleic acid molecule comprising SEQ ID NO:18 having a G at position 980; (kk) an oligonucleotide or peptide nucleic acid that hybridizes under high stringency conditions to a nucleic acid molecule comprising SEQ ID NO:19 having a C at position 301 but not to a nucleic acid molecule comprising SEQ ID NO:19 having a G at position 301; (ll) an oligonucleotide or peptide nucleic acid that hybridizes under high stringency conditions to a nucleic acid molecule comprising SEQ ID NO:19 having a G at position 301 but not to a nucleic acid molecule comprising SEQ ID NO:19 having a C at position 301; (mm) an oligonucleotide or peptide nucleic acid that hybridizes under high stringency conditions to a nucleic acid molecule comprising SEQ ID NO:20 having a G at position 301 but not to a nucleic acid molecule comprising SEQ ID NO:20 having an A at position 301; (nn) an oligonucleotide or peptide nucleic acid that hybridizes under high stringency conditions to a nucleic acid molecule comprising SEQ ID NO:20 having an A at position 301 but not to a nucleic acid molecule comprising SEQ ID NO:20 having a G at position 301; (oo) an oligonucleotide or peptide nucleic acid that hybridizes under high stringency conditions to a nucleic acid molecule comprising SEQ ID NO:21 having an A at position 301 but not to a nucleic acid molecule comprising SEQ ID NO:21 having a G at position 301; (pp) an oligonucleotide or peptide nucleic acid that hybridizes under high stringency conditions to a nucleic acid molecule comprising SEQ ID NO:21 having a G at position 301 but not to a nucleic acid molecule comprising SEQ ID NO:21 having an A at position 301; (qq) an oligonucleotide or peptide nucleic acid that hybridizes under high stringency conditions to a nucleic acid molecule comprising SEQ ID NO:22 having an A at position 301 but not to a nucleic acid molecule comprising SEQ ID NO:22 having a G at position 301; (rr) an oligonucleotide or peptide nucleic acid that hybridizes under high stringency conditions to a nucleic acid molecule comprising SEQ ID NO:22 having a G at position 301 but not to a nucleic acid molecule comprising SEQ ID NO:22 having an A at position 301; (ss) an oligonucleotide or peptide nucleic acid that hybridizes under high stringency conditions to a nucleic acid molecule comprising SEQ ID NO:23 having a C at position 301 but not to a nucleic acid molecule comprising SEQ ID NO:23 having a T at position 301; (tt) an oligonucleotide or peptide nucleic acid that hybridizes under high stringency conditions to a nucleic acid molecule comprising SEQ ID NO:23 having a T at position 301 but not to a nucleic acid molecule comprising SEQ ID NO:23 having a C at position 301; (uu) an oligonucleotide or peptide nucleic acid capable of hybridizing under high stringency conditions to a nucleic acid molecule comprising a first allele for a given polymorphism selected from the polymorphisms listed in TABLE 1D but not capable of hybridizing under high stringency conditions to a nucleic acid molecule comprising a second allele for the given polymorphism selected from the polymorphisms listed in TABLE 1D; and (vv) an oligonucleotide or peptide nucleic acid capable of hybridizing under high stringency conditions to a nucleic acid molecule comprising the second allele for a given polymorphism selected from the polymorphisms listed in TABLE 1D but not capable of hybridizing under high stringency conditions to a nucleic acid molecule comprising the first allele for the given polymorphism selected from the polymorphisms listed in TABLE 1D.

In accordance with further aspects of the invention, an array of oligonucleotides or peptide nucleic acids attached to a solid support is provided; the array comprising two or more of the oligonucleotides or peptide nucleic acids set out herein.

In accordance with further aspects of the invention, a composition is provided including an addressable collection of two or more oligonucleotides or peptide nucleic acids; the two or more oligonucleotides or peptide nucleic acids consisting essentially of two or more nucleic acid molecules set out in SEQ ID NO:1-23 or compliments; fragments; variants; or analogs thereof.

The oligonucleotides or peptide nucleic acids may further include one or more of the following: a detectable label; a quencher; a mobility modifier; a contiguous non-target sequence situated 5′ or 3′ to the target sequence or 5′ and 3′ to the target sequence.

The one or more polymorphic sites in linkage disequilibrium thereto is selected from one or more of the polymorphic sites listed in TABLE 1B.

The oligonucleotides or peptide nucleic acids may further include one or more of the following: a detectable label; a quencher; a mobility modifier; a contiguous non-target sequence situated 5′ or 3′ to the target sequence or 5′ and 3′ to the target sequence. The oligonucleotides or peptide nucleic acids may alternatively be of about 10 to about 400 nucleotides, about 15 to about 300 nucleotides. The oligonucleotides or peptide nucleic acids may alternatively be of about 20 to about 200 nucleotides, about 25 to about 100 nucleotides. The oligonucleotides or peptide nucleic acids may alternatively be of about 20 to about 80 nucleotides, about 25 to about 50 nucleotides.

Oligonucleotides or peptide nucleic acids; arrays; addressable collections of oligonucleotides or peptide nucleic acids and a computer readable medium comprising a plurality of digitally encoded genotype correlations are provided as described herein. There may be two or more oligonucleotides or peptide nucleic acids. Alternatively; there may be three or more oligonucleotides or peptide nucleic acids; four or more oligonucleotides or peptide nucleic acids or five or more oligonucleotides or peptide nucleic acids; or six or more oligonucleotides or peptide nucleic acids; or seven or more oligonucleotides or peptide nucleic acids; or eight or more oligonucleotides or peptide nucleic acids; or nine or more oligonucleotides or peptide nucleic acids or ten or more oligonucleotides or peptide nucleic acids.

Sequence variations may be assigned to a gene if mapped within 2 kb or more of an mRNA sequence feature. In particular; such a sequence may extend many kilobases (kb) from a SERPINE1 or PROC gene and into neighbouring genes; where the LD within a region is strong.

### BRIEF DESCRIPTION OF THE DRAWINGS

FIG. 1.1.1 shows a plot of mean survival (N_{survived}/Nₜₒₜₐₗ) by SERPINE1 rs7242 genotype for XIGRIS™-treated and placebo-treated subjects in the PROWESS study (All subjects).

FIG. 1.1.2a shows a plot of mean survival (N_{survived}/Nₜₒₜₐₗ) by SERPINE1 rs7242 genotype for XIGRIS™-treated and placebo-treated subjects in the PROWESS study (All subjects APACHE II ≧25).

FIG. 1.1.2b shows a plot of PAI-I levels by rs7242 genotype (mean and 95% confidence interval) for placebo-treated subjects in the PROWESS study (All subjects APACHE II ≧25).

FIG. 1.1.2c shows a plot of PAI-I levels by rs7242 genotype (mean and 95% confidence interval) for XIGRIS™-treated subjects in the PROWESS study (All subjects APACHE II ≧25).

FIG. 1.1.2d shows a plot of PC levels by rs7242 genotype (mean and 95% confidence interval) for Placebo-treated subjects in the PROWESS study (All subjects APACHE II ≧25).

FIG. 1.1.2e shows a plot of PC levels by rs7242 genotype (mean and 95% confidence interval) for XIGRIS™-treated subjects in the PROWESS study (All subjects APACHE II ≧25).

FIG. 1.2.1 shows a plot of mean survival (N_{survived}/Nₜₒₜₐₗ) by SERPINE1 rs7242 genotype for XIGRIS™-treated and control subjects in the SPH severe sepsis cohort.

FIG. 1.2.2 shows a plot of mean survival (N_{survived}/Nₜₒₜₐₗ) by SERPINE1 rs2070682 genotype for XIGRIS™-treated and control subjects in the SPH severe sepsis cohort.

FIG. 2.1.1 shows a plot of mean survival (N_{survived}/Nₜₒₜₐₗ) by SERPINE1 rs2227684 genotype for XIGRIS™-treated and placebo-treated subjects in the PROWESS study (All subjects).

FIG. 2.2.1 shows a plot of mean survival (N_{survived}/Nₜₒₜₐₗ) by SERPINE1 rs11178 genotype for XIGRIS™-treated and placebo-treated subjects in the PROWESS study (All subjects).

FIG. 2.3.1 shows a plot of mean survival (N_{survived}/Nₜₒₜₐₗ) by SERPINE1 rs2227706 genotype for XIGRIS™-treated and placebo-treated subjects in the PROWESS study (All subjects).

FIG. 3.1.1 shows a plot of PAI-I levels by rs7242/rs2069912 combined genotype (mean) for Placebo-treated subjects in the PROWESS study (All subjects APACHE II ≧25).

FIG. 3.1.2 shows a plot of PAM levels by rs7242/rs2069912 combined genotype (mean) for XIGRIS™-treated subjects in the PROWESS study (All subjects APACHE II ≧25).

FIG. 3.1.3. shows a plot of mortality by rs7242/rs2069912 combined genotype of matched control and XIGRIS™-treated patients subjects in the SPH cohort. The numbers within the bars represent the number of subjects within the group.

FIG. 3.1.4. shows a plot of mortality by rs7242/rs2069912 combined genotype of placebo- and XIGRIS™-treated patients subjects with APACHE II ≧25 in the PROWESS cohort study. The numbers within the bars represent the number of subjects within the group.

FIG. 4.3.2 shows in Panel (A) a plot of the mean ratio of PAI-1/PROC protein levels over days 1-5, in Panel (B) a plot of 28-day mortality, and in Panel (C) a plot of the distribution of serious adverse events; all by combined rs7242/rs2069912 genotype, for Placebo-treated (left) and XIGRIS™-treated (right) subjects in the PROWESS study (All subjects APACHE II ≧25). Error bars represent standard error.

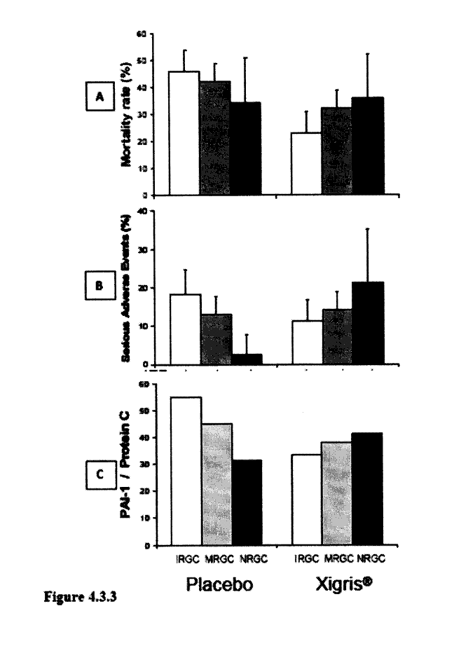

### DETAILED DESCRIPTION

### 1. Definitions

In the description that follows, a number of terms are used extensively, the following definitions are provided to facilitate understanding of the invention.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). A “Nucleotide” is generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, while a nucleotide represented by the symbol D may be either A or G or T, while a nucleotide represented by the symbol B may be either G or C or T, and a nucleotide represented by the symbol H may be either A or C or T.

A “polymorphic site” or “polymorphism site” or “polymorphism” or “single nucleotide polymorphism site” (SNP site) or single nucleotide polymorphism” (SNP) as used herein is the locus or position with in a given sequence at which divergence occurs. A “polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, introns or untranslated regions) of genes. Polymorphisms may occur at a single nucleotide site (SNPs) or may involve an insertion or deletion as described herein.

A “risk genotype” or “risk allele” as used herein refers to an allelic variant (genotype) at one or more polymorphic sites within the SERPINE1 and PROC gene sequences described herein as being indicative of a decreased likelihood of recovery from an inflammatory condition or an increased risk of having a poor outcome. The risk genotype may be determined for either the haploid genotype or diploid genotype, provided that at least one copy of a risk allele is present. Risk genotype may be an indication of an increased risk of not recovering from an inflammatory condition. Subjects having one copy (heterozygotes) or two copies (homozygotes) of the risk allele are considered to have the “risk genotype” even though the degree to which the subjects risk of not recovering from an inflammatory condition may increase, depending on whether the subject is a homozygote rather than a heterozygote as shown herein.

A “decreased risk genotype” as used herein refers to an allelic variant (genotype) at one or more polymorphic sites within the SERPINE1 and PROC gene sequences described herein as being indicative of an increased likelihood of recovery from an inflammatory condition or a decreased risk of having a poor outcome. The decreased risk genotype may be determined for either the haploid genotype or diploid genotype, provided that at least one copy of a risk allele is present. Decreased risk genotype may be an indication of an increased likelihood of recovering from an inflammatory condition. As described herein subjects having two copies (homozygotes) of the decreased risk allele are considered to have the “decreased risk genotype” (for example rs7242 GG).

An “improved response genotype” (IRG) or improved response polymorphic variant reduced adverse response genotype as used herein refers to an allelic variant or genotype at one or more polymorphic sites from one or both of serpin peptidase inhibitor, Glade E, member 1 (SERPINE1), and Protein C (PROC) as described herein as being predictive of a subject's increased likelihood of survival or of an improved survival prognosis in response to treatment with an anti-inflammatory agent or an anti-coagulant agent, or a polymorphic site in linkage disequilibrium thereto or a reduction in serious adverse events or adverse events in response to treatment with an anti-inflammatory agent or an anti-coagulant agent as described herein.

An “non-response genotype” (NRG) or non-response polymorphic variant or adverse response genotype as used herein refers to an allelic variant or genotype at one or more polymorphic sites from one or both of the serpin peptidase inhibitor, clade E, member 1 (SERPINE1), and Protein C (PROC) as described herein as being predictive of a subject's decreased likelihood of survival or an reduced survival prognosis in response to treatment with an anti-inflammatory agent or an anti-coagulant agent, or a polymorphic site in linkage disequilibrium thereto or an increase in serious adverse events or adverse events in response to treatment with an anti-inflammatory agent or an anti-coagulant agent as described herein.

An “Improved Response Genotype Combination” (IRGC) as used herein refers to an allelic variant or genotype at one or more polymorphic sites selected from SERPINE1 and PROC or a polymorphic site in linkage disequilibrium thereto as described herein, wherein the genotype combination(s) is predictive of subjects who have an increased likelihood of survival or an improved survival prognosis in response to treatment with an anti-inflammatory agent or an anti-coagulant agent or a reduction in serious adverse events or adverse events in response to treatment with an anti-inflammatory agent or an anti-coagulant agent as described herein. An IRGC may be selected from one or more of the following: rs7242 GT/rs2069912 CC; rs7242 GT/rs2069912 CT; rs7242 TT/rs2069912 CC; rs7242 TT/rs2069912 CT; rs2070682 CT/rs2069912 CC; rs2070682 CT/rs2069912 CT; rs2070682 TT/rs2069912 CC; rs2070682 TT/rs2069912 CT; rs11178 CT/rs2069912 CC; rs11178 CT/rs2069912 CT; rs11178 TT/rs2069912 CC; rs11178 TT/rs2069912 CT; rs2227706 AG/rs2069912 CC; rs2227706 GG/rs2069912 CT; rs2227706 AG/rs2069912 CT; rs2227706 GG/rs2069912 CC; rs2227684 AG/rs2069912 CC; rs2227684 GG/rs2069912 CT; rs2227684 AG/rs2069912 CT; rs2227684 GG/rs2069912 CC; or one or more polymorphic sites in linkage disequilibrium thereto.

An “Non Response Genotype Combination” (NRGC) as used herein refers to an allelic variant or genotype at one or more polymorphic sites from one or both of SERPINE1 and PROC or a polymorphic site in linkage disequilibrium thereto as described herein, wherein the genotype combination(s) is predictive of subjects that are non responders to treatment with an anti-inflammatory agent or an anti-coagulant agent or an increase in serious adverse events or adverse events in response to treatment with an anti-inflammatory agent or an anti-coagulant agent as described herein. An NRGC may be selected from one or more of the following: rs7242 GG/rs2069912 TT; rs2070682 CC/rs2069912 TT; rs11178 CC/rs2069912 TT; rs2227706 AA/rs2069912 TT; rs2227684 AA/rs2069912 TT; or one or more polymorphic sites in linkage disequilibrium thereto.

An “Mixed Response Genotype Combination” (MRGC) as used herein refers to an allelic variant or genotype at one or more polymorphic sites from one or both of SERPINE1 and PROC or a polymorphic site in linkage disequilibrium thereto as described herein. An MRGC may be selected from one or more of the following: rs7242 GT/rs2069912 TT; rs7242 GG/rs2069912 CC; rs7242 GG/rs2069912 CT; rs7242 TT/rs2069912 TT; rs2070682 CT/rs2069912 TT; rs2070682 CC/rs2069912 CC; rs2070682 CC/rs2069912 CT; rs2070682 TT/rs2069912 TT; rs11178 CT/rs2069912 TT; rs11178 CC/rs2069912 CC; rs11178 CC/rs2069912 CT; rs11178 TT/rs2069912 TT; rs2227706 AG/rs2069912 TT; rs2227706 AA/rs2069912 CC; rs2227706 AA/rs2069912 CT; rs2227706 GG/rs2069912 TT; rs2227684 AG/rs2069912 TT; rs2227684 AA/rs2069912 CC; rs2227684 AA/rs2069912 CT; rs2227684 GG/rs2069912 TT; or one or more polymorphic sites in linkage disequilibrium thereto and may represent an increased likelihood of survival or an improved survival prognosis.

A “clade” is a group of haplotypes that are closely related phylogenetically. For example, if haplotypes are displayed on a phylogenetic (evolutionary) tree a clade includes all haplotypes contained within the same branch.

As used herein “haplotype” is a set of alleles of closely linked loci on a chromosome that tend to be inherited together. Such allele sets occur in patterns, which are called haplotypes. Accordingly, a specific SNP or other polymorphism allele at one SNP site is often associated with a specific SNP or other polymorphism allele at a nearby second SNP site or other polymorphism site. When this occurs, the two SNPs or other polymorphisms are said to be in LD because the two SNPs or other polymorphisms are not just randomly associated (i.e. in linkage equilibrium).

In general, the detection of nucleic acids in a sample depends on the technique of specific nucleic acid hybridization in which the oligonucleotide is annealed under conditions of a stringency sufficient to distinguish a single nucleotide mismatch to nucleic acids in the sample, and the successfully annealed oligonucleotides are subsequently detected (see for example Spiegelman, S., Scientific American, Vol. 210, p. 48 (1964)). The specificity depends on the conditions used for hybridization, the oligonucleotide length, base composition and position of mismatches (if any). The term “high stringency” hybridization as used herein refers to conditions that provide for specificity with relatively short probes as is known in the art and is relied upon for the success of numerous techniques routinely performed by molecular biologists and is relied upon for the success of numerous techniques routinely performed by molecular biologists, such as high-stringency PCR, DNA sequencing, single strand conformational polymorphism analysis, and in situ hybridization. In contrast to Northern and Southern hybridizations, these aforementioned techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

“Oligonucleotides” as used herein are variable length nucleic acids, which may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI D A. et al. Proc Nat Acad Sci (1995) 92(17):7912-5; MCGALL G. et al. PNAS (1996) 93(24):13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired. As used herein, “oligonucleotides” also includes various analogs that are commonly used in the art, including oligonucleotides synthesized with modified nucleic acids, such as locked nucleic acids (LNA) (as described in, for example, U.S. Pat. No. 6,268,490), and also oligonucleotides having modified backbones.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

An “addressable collection” as used herein is a combination(s) of nucleic acid molecules or peptide nucleic acids capable of being detected by, for example, the use of hybridization techniques or by any other means of detection known to those of ordinary skill in the art. A DNA microarray would be considered an example of an “addressable collection”.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination(s). “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like SNPs, are often useful in tracking meiotic recombination events as positional markers on chromosomes.

The pattern of a set of markers along a chromosome is referred to as a “Haplotype”. Accordingly, groups of alleles on the same small chromosomal segment tend to be transmitted together. Haplotypes along a given segment of a chromosome are generally transmitted to progeny together unless there has been a recombination event. Absent a recombination event, haplotypes can be treated as alleles at a single highly polymorphic locus for mapping.

Furthermore, the preferential occurrence of a disease gene in association with specific alleles of linked markers, such as SNPs or other polymorphisms, is called “Linkage Disequilibrium” (LD). This sort of disequilibrium generally implies that most of the disease chromosomes carry the same mutation and the markers being tested are relatively close to the disease gene(s).

For example, in SNP-based association analysis and LD mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs may be tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For an LD based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs) and short tandem repeats (STRs).

In population genetics linkage disequilibrium refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as position 201 of SEQ ID NO:1 (see Akey, J. et al. Eur J Hum Genet (2001) 9:291-300; and Zhang, K. et al. (2002). Am J Hum Genet. 71:1386-1394). This viewpoint is further substantiated by Khoury et al. ((1993). Fundamentals of Genetic Epidemiology. New York: Oxford University Press at p. 160) who state, “Whenever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for r²≧0.5) are potentially useful in predicting the identity of an allele of interest (i.e. associated with the condition of interest). A high degree of linkage disequilibrium may be represented by an absolute value for r²≧0.6. Alternatively, a high degree of linkage disequilibrium may be represented by an absolute value for r²≧0.7 or by an absolute value for r²≧0.8. Additionally, a high degree of linkage disequilibrium may be represented by an absolute value for r²≧0.85 or by an absolute value for r²≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably. For example, in the population from which the tagged SNPs were identified from the SNP identified by rs7242 is in “linkage disequilibrium” with the SNP identified by rs11178, whereby when the genotype of by rs7242 is G the genotype of rs11178 is C. Similarly, when the genotype of by rs7242 is T the genotype of rs11178 is T. Accordingly, the determination of the genotype at by rs7242 will provide the identity of the genotype at rs11178 or any other locus in “linkage disequilibrium” therewith. Particularly, where such a locus is has a high degree of linkage disequilibrium thereto.

LD is useful for genotype-phenotype association studies. For example, if a specific allele at one SNP site (e.g. “A”) is the cause of a specific clinical outcome (e.g. call this clinical outcome “B”) in a genetic association study then, by mathematical inference, any SNP (e.g. “C”) which is in significant LD with the first SNP, will show some degree of association with the clinical outcome. That is, if A is associated (˜) with B, i.e. A˜B and C˜A then it follows that C˜B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on LD between A and C.

Until the mechanism underlying the genetic contribution to a specific clinical outcome is fully understood, LD helps identify potential candidate causal SNPs and also helps identify a range of SNPs that may be clinically useful for prognosis of clinical outcome or of treatment effect. If one SNP within a gene is found to be associated with a specific clinical outcome, then other SNPs in LD will also have some degree of association and therefore some degree of prognostic usefulness. By way of prophetic example, if multiple polymorphisms were tested for individual association with an improved response to XIGRIS™ administration in our SIRS/sepsis/septic shock cohort of ICU subjects, wherein the multiple polymorphisms had a range of LD with SERPINE1 polymorphism rs7242 and it was assumed that rs7242 was the causal polymorphism, and we were to order the polymorphisms by the degree of LD with rs7242, we would expect to find that polymorphisms with high degrees of LD with rs7242 would also have a high degree of association with this specific clinical outcome. As LD decreased, we would expect the degree of association of the polymorphism with an improved response XIGRIS™ receptor agonist administration to also decrease. Accordingly, logic dictates that if A˜B and C˜A, then C˜B. That is, any polymorphism, whether already discovered or as yet undiscovered, that is in LD with one of the improved response genotypes described herein will likely be a predictor of the same clinical outcomes that rs7242 is a predictor of. The similarity in prediction between this known or unknown polymorphism and rs7242 would depend on the degree of LD between such a polymorphism and rs7242.

Polymorphic sites have been identified as in SERPINE1 and PROC genes (see TABLE 1A). Furthermore, the polymorphisms in TABLE 1A are linked to (in LD with) numerous polymorphism as set out in TABLE 1B below and may also therefore be indicative of subject prognosis.

TABLE 1A
Polymorphisms in the SERPINE1 and PROC genes genotyped in a cohort of
critically ill Subjects with severe sepsis. Minor Allele Frequencies (MAFs) for
Caucasians were taken from the Seattle SNPs PGA (http://pga.gs.washington.edu/).

| | | | March 2006 | | |
|---|---|---|---|---|---|
| | | | Chromosomal | | Minor |
| Polymorphism Name | | | position | Minor | Allele |
| (RSID Alleles) | Official Gene Name | rs# | (Build 36) | allele | Frequency |
| SERPINE1 rs7242 G/T | serpin peptidase | rs7242 | 100568165 | G | 0.326 |
| | inhibitor, clade E, | | | | |
| | member 1 | | | | |
| | (SERPINE1) | | | | |
| PROC rs2069912 C/T | Protein C (PROC) | rs2069912 | 127894661 | C | 0.261 |

TABLE 1B
Polymorphisms in linkage disequilibrium with those listed in TABLE 1A above,
as identified using the Haploview program (BARRETT JC. et al. Bioinformatics (2005)
21(2): 263-5 (http://www.broad.mit.edu/mpg/haploview/)) and the LD function in the Genetics
Package in R (R Core Development Group, 2005 - R Development Core Team (www.R-project.org).
Linkage Disequilibrium between markers was defined using r² whereby all
SNPs available on Hapmap.org (phase II) (cohort H), all SNPs genotyped internally using the
Illumina Goldengate assay (cohort I) and all SNPs genotyped by the Seattle SNPs PGA on
http://pga.gs.washington.edu (cohort S) in our genes of interest were included. A minimum r²
of 0.5 was used as the cutoff to identify LD SNPs. The genes are identified, along with the
alleles, rs designation and the chromosomal position (March 2006 Build 36).

| | | | | | | | RSIDs of |
|---|---|---|---|---|---|---|---|
| | Tag | Tag | Polymorphism | | LD | Polymorphism | Polymorphism |
| Gene | SNP | Polymorphisms | RSID | Cohort | Allele | in LD | in LD |
| SERPINE1 | G | 100568165 | rs7242 | S | C | 100567804 | rs11178 |
| | | | | S | C | 100569464 | rs2227703 |
| | | | | S | A | 100570015 | rs2227706 |
| | | | | S | T | 100561236 | rs2227662 |
| | | | | S | G | 100561277 | rs2227673 |
| | | | | S | T | 100563022 | rs2227679 |
| | | | | S/H | A | 100563651 | rs2227684 |
| | | | | S/I/H | C | 100563987 | rs2070682 |
| | | | | S | G | 100565044 | rs2227686 |
| | | | | S | T | 100565047 | rs2227687 |
| | | | | H | C | 100574122 | rs757716 |
| | | | | H | T | 100570456 | rs13238709 |
| | | | | H | G | 100571842 | rs11560324 |
| PROC | C | 127894661 | rs2069912 | S | A | 127895796 | rs2069918 |
| | | | | S | T | 127898845 | rs1518759 |
| | | | | S | T | 127902158 | rs2069933 |
| | | | | S | T | 127897988 | rs971207 |
| | | | | S | A | 127896691 | rs973760 |
| | | | | S | A | 127894742 | rs2069914 |
| | | | | S | G | 127896308 | rs2069921 |
| | | | | I | G | 127894729 | rs2069913 |

It will be appreciated by a person of skill in the art that further linked polymorphic sites and combined polymorphic sites may be determined. A haplotype of the SERPINE1 and PROC genes can be created by assessing polymorphisms SERPINE1 and PROC—genes in normal subjects using a program that has an expectation maximization algorithm. A constructed haplotype of SERPINE1 and PROC genes may be used to find combinations of SNPs that are subjects using a program that has an expectation maximization algorithm. A constructed haplotype of SERPINE1 and PROC genes may be used to find combinations of SNPs that are in LD with the tag SNPs (tSNPs) identified herein. Accordingly, the haplotype of an individual could be determined by genotyping other SNPs or other polymorphisms that are in LD with the tSNPs identified herein. Single polymorphic sites or combined polymorphic sites in LD may also be genotyped for assessing subject response to XIGRIS™ treatment.

It will be appreciated by a person of skill in the art that the numerical designations of the positions of polymorphisms within a sequence are relative to the specific sequence. Also the same positions may be assigned different numerical designations depending on the way in which the sequence is numbered and the sequence chosen, as illustrated by the alternative numbering of the equivalent polymorphism (rs7242), whereby the same polymorphism identified G/T at position 2006 of the NM_{—}000602.1 (GI:10835158), which corresponds to position 301 of SEQ ID NO:1. Furthermore, sequence variations within the population, such as insertions or deletions, may change the relative position and subsequently the numerical designations of particular nucleotides at and around a polymorphic site.

Polymorphic sites in SEQ ID NO:1-2 and SEQ ID NO:3-23 are identified by their variant designation (i.e. M, W, Y, S, R, K, V, B, D, H or by “−” for a deletion, a “+” or “G” etc. for an insertion).

TABLE 1C below shows the flanking sequences for a SERPINE1 SNP and PROC SNP giving their ‘rs’ designations and corresponding SEQ ID NO designations. Each polymorphism is at position 301 within the flanking sequence, unless otherwise indicated, and identified in bold and underlined.

TABLE 1C

| | | SEQ | |
|---|---|---|---|
| | | ID | |
| GENE | SNP RSID | NO: | POLYMORPHIC AND FLANKING SEQUENCE |
| SERPINE1 | rs7242 | 1 | ACATTGCCATCACTCTTGTACTGCCTGCCACCGCGGAGGAGGCTGGTGAC |
| | | | AGGCCAAAGGCCAGTCGAACAAACACCCTTTCATCTCAGAGTCCACTGTG |
| | | | GCACTGGCCACCCCTCCCCAGTACAGGGGTGCTGCAGGTGGCAGAGTGAA |
| | | | TGTCCCCCATCATGTGGCCCAACTCTCCTGGCCTGGCCATCTCCCTCCCC |
| | | | AGAAACAGTGTGCATGGGTTATTTTGGAGTGTAGGTGACTTGTTTACTCA |
| | | | TTGAAGCAGATTTCTGCTTCCTTTTATTTTTATAGGAATAGAGGAAGAAA |
| | | | KGTCAGATGCGTGCCCAGCTCTTCACCCCCCAATCTCTTGGTGGGGAGGG |
| | | | GTGTACCTAAATATTTATCATATCCTTGCCCTTGAGTGCTTGTTAGAGAG |
| | | | AAAGAGAACTACTAAGGAAAATAATATTATTTAAACTCGCTCCTAGTGTT |
| | | | TCTTTGTGGTCTGTGTCACCGTATCTCAGGAAGTCCAGCCACTTGACTGG |
| | | | CACACACCCCTCCGGACATCCAGCGTGACGGAGCCCACACTGCCACCTTG |
| | | | TGGCCGCCTGAGACCCTCGCGCCCCCCGCGCCCCCCGCGCCCCTCTTTTT |
| | | | C |
| PROC | rs2069912 | 2 | CCCCTTTCCTGGTCTCCACAGCCAACGGGAGGAGGCCATGATTCTTGGGG |
| | pos = 201 | | AGGTCCGCAGGACACATGGGCCCCTAAAGCCACACCAGGCTGTTGGTTTC |
| | | | ATTTGTGCCTTTATAGAGCTGTTTATCTGCTTGGGACCTGCACCTCCACC |
| | | | CTTTCCCAAGGTGCCCTCAGCTCAGGCATACCCTCCTCTAGGATGCCTTT |
| | | | YCCCCCATCCCTTCTTGCTCACACCCCCAACTTGATCTCTCCCTCCTAAC |
| | | | TGTGCCCTGCACCCAAGACAGACACTTCACAGAGCCCAGGAGACACCTGG |
| | | | GGACCCTTCCTGGGTGATAGGTCTGTCTATCCTCCAGGTGTCCCTGCCCA |
| | | | AGGGGAGAAGCATGGGGAATACTTGGTTGGGGGAGGAGAGGAAGACTGGG |
| | | | G |

An “rs” prefix designates a SNP in the database is found at the NCBI SNP database (http://www.ncbi.nlm.nih.gov/entrez/query.fcgi?db=Snp). The “rs” numbers are the NCBI | rsSNP ID form.

TABLE 1D below shows the flanking sequences for a selection of SERPINE1 and PROC associated gene SNPs providing their rs designations and corresponding SEQ ID NO designations. Each polymorphism is at position 301 within the flanking sequence, unless otherwise indicated, and identified in bold and underlined.

TABLE 1D

| | | SEQ | |
|---|---|---|---|
| | | ID | |
| GENE | SNP RSID | NO: | POLYMORPHIC AND FLANKING SEQUENCE |
| SERPINE1 | rs11178 | 3 | TCCGAAGAAAAGAATTTTAGTGTTAATGACTCTTTCTGAAGGAAGAGAAG |
| | | | ACATTTGCCTTTTGTTAAAAGATGGTAAACCAGATCTGTCTCCAAGACCT |
| | | | TGCCCTCTCCTTGGAGGACCTTTAGGTCAAACTCCCTAGTCTCCACCTGA |
| | | | GACCCTGGGAGAGAAGTTTGAAGCACAACTCCCTTAAGGTCTCCAAACCA |
| | | | GACGGTGACGCCTGCGGGACCATCTGGGGCACCTGCTTCCACCCGTCTCT |
| | | | CTGCCCACTCGGGTCTGCAGACCTGGTTCCCACTGAGGCCCTTTGCAGGA |
| | | | YGGAACTACGGGGCTTACAGGAGCTTTTGTGTGCCTGGTAGAAACTATTT |
| | | | CTGTTCCAGTCACATTGCCATCACTCTTGTACTGCCTGCCACCGCGGAGG |
| | | | AGGCTGGTGACAGGCCAAAGGCCAGTGGAAGAAACACCCTTTCATCTCAG |
| | | | AGTCCACTGTGGCACTGGCCACCCCTCCCCAGTACAGGGGTGCTGCAGGT |
| | | | GGCAGAGTGAATGTCCCCCATCATGTGGCCCAACTCTCCTGGCCTGGCCA |
| | | | TCTCCCTCCCCAGAAACAGTGTGCATGGGTTATTTTGGAGTGTAGGTGAC |
| | | | T |
| SERPINE1 | rs757716 | 4 | TTTGGAAGGCCGAGGCAAGTGAATCACCTGAGTCCAGGAGTTCGAGACCA |
| | | | GCCTGGGCAACATGGCAAAACCTCGTCTCCACAAAAAAAAATACAAAAAT |
| | | | GAGCTGGGCGTGGTGGCGGGCGCCAGTAGTCCCAGCTACTTTGGAGGCTG |
| | | | AGGTGGGAGGATTGCCTGAGCCAAGGAGGTCGAGGTTGCAGTGAGCCAAG |
| | | | ATCACGCCACTACACTCCAGCCTGGGGTGACAGAGCAAGACCCCGGCTCA |
| | | | AAAAAAAAAAAAAAAAAGTGCTGCCCTCACGTGCCAGTCATCAGCAGCTT |
| | | | STGTGTTGCCACCAGCCGCGGGGCCAGCCTGAACACTTGCTTCCTGAACC |
| | | | AGGTCTTTGATCTCCTCGCCACCAGAGGTCGCTTTCGCTCCAGTGGAAAG |
| | | | CTCTTCCCTGAAGCAAACCAGCTGTGGCTTCCACCAAAATCTTGCCAAGT |
| | | | TGGAACTCCATTCAATTTAACGAGTATTTATTTAAAAGCTGACCTTTAGA |
| | | | CTGGGTACTGTGAAGCATTAAAAAGAAAGTGTCAGCTATTCTTAATAGCA |
| | | | GATATCTAATGACAAGGGACATCAAAAAGCTGGGGAAAAAAAGCTATGCT |
| | | | G |
| SERPINE1 | rs2070682 | 5 | TCTTCACAGCTGAGTTCACCACGCCCGATGGCCATTACTACGACATCCTG |
| | | | GAACTGCCCTACCACGGGGACACCCTCAGCATGTTCATTGCTGCCCCTTA |
| | | | TGAAAAAGAGGTGCCTCTCTCTGCCCTCACCAACATTCTGAGTGCCCAGC |
| | | | TCATCAGCCACTGGAAAGGCAACATGACCAGGCTGCCCCGCCTCCTGGTT |
| | | | CTGCCCAAGTAAGCCACCCCGCTATCTCCCCGACCTACCAACCCCTCTCT |
| | | | CCTGGCTCCCTAAAGTCACCCCCCCCAGGTTGAATTTCCCAGATCTGTGA |
| | | | YGCTTGCAGGACATGCATGTGTGGGAGGCTGATGGGAAACTGTGGCCTGG |
| | | | GTTTGATTATGAGTCTTGCAATCATCCCTCCCCCTGTTTCTGCTGGAGGG |
| | | | CAGGGGACAGCTCTTCCTGACCACACCCCCACATTGACTATCCCCAGAAT |
| | | | ACCCAGCAAAAGCCCCCAAAAGGAGAGTCAGAGAAATGAGGGAGGTGGGG |
| | | | GCCCAATCAGTCCACATCTACTTAGGGTCGCCCCATCAGCACTTCCATCC |
| | | | CCAACCCTTTCAAGTCAACATCCAAACAAAAGAAATCACTTCCAAGGACG |
| | | | G |
| SERPINE1 | rs2227662 | 6 | TTGAGTCCAGCAGTTCAAGGCTGCAGTGAGCTATGTTTGCACCACCACAC |
| | | | TCCAGCCTGAGTCACAGAACAAGACCTCATCTCTAAAAAACAAACAAAAA |
| | | | CCAAATCCACATATCCTAAAAAATGCTCCTTTTCAGCATTCTCTTCTCTA |
| | | | TGGACAAAGGGCTGGATGCTTTAAGAACCAAATCTTAGGCTGGGCACGGT |
| | | | GGCTCACGCCTCTAATCCTAGCACTTTGAGAGGCCAAGGCGGGCAGATTG |
| | | | CCTGAGCACAGGAGTTCGAGACCAGCCTGGCCAACATGGTGAAACCCTGT |
| | | | YTCTGTCAAAAATACAAAAAATTAGCCAGGTGTGTTGGCGCGTGCCTATA |
| | | | ATCCCAGCTGCTCCGGAGGATGAGGTTCAAAGAATCACTTGAACCCGGGA |
| | | | GGCAGAGGCTGCAGTGAGCTGAGATCATGCCACTGCACTCCAGCCTGGGT |
| | | | GACAGAGCAAGACTTTGTCTCCAAAAAAAGGAACTAGACGGGTTCATTTA |
| | | | AACCCCTGACTGCAGCCCTTTGACATACATCCAATTGAGGACTGGGGACT |
| | | | CCGGGAAACATCTAAAAGGCTTAAAAACTTTGTCTAACTTCAGCCGGGCA |
| | | | T |
| SERPINE1 | rs2227673 | 7 | CCAAGCAGGAGACATCAGGATAATGGGAACAGAAGACAGGAGGTTTATCC |
| | pos = 468 | | CATGAAGGATGAAGAAGCTGAAATCCAGAGATTCCCTCAGGGCCACATTT |
| | | | GTCCACCTGACTCCAGGGTCTCATCTTCGTGTGTTGCTAGTGTGATTACC |
| | | | TGGGGATGAGAAATCCTGCTGGGGGAGTTGAGGTTAAGAGGATGAGGACT |
| | | | CCAGGTGCTGTGGCTCACGCCTGTAATCCCAGCACTTTGGGAGGCCAAGG |
| | | | CAGGTGGATCAGGAGTTTGAGGTCAGGAGTTTGAGACCAGCCTGGCCAAC |
| | | | ATGGTGAAACCCTGTCTCTACTAAAAATGCAAAAATTAGCCAGGTGTGGT |
| | | | GGCAGGCGCCTGTAATCCCAGCTACTCGGGAGGCTGAGGCAGGAGAATCA |
| | | | CTTGAGCCCGGGAGGTGGAGGTTGCAGTGAGCCGAACGAAATTGAGCCAC |
| | | | TTCACCCCAGCCTGGGCRAAAGAGTGAAATTCCATTCAAAAAAAAAAAAA |
| | | | AAAAAAAAAGGATGAGGACTGGGATGAACTGGTGGCTGGGTGTGGGGAAA |
| | | | ATGGAAGTGAAGGAAGGCCAAAAGAGACAGAGAAGGCCTGGCGCGGCGAC |
| | | | TCACGCCTATAATCCCAGCACTTTGGGAGGCTGAGAAGGGGGATTGCTTG |
| | | | AGGCCAGAAGTTGAATACCAGTCTGGGCAGCATAGCAAGACCCTGCCTCT |
| | | | ACAAAAAAAAAATTTTTTTTAATTAGCCAGGCTTGGTGACATGCATCTGT |
| | | | AGTCTACTCAAGAAGCTGAGGTGAGGCCAGGCACGGTGGCTCACCCCTGT |
| | | | ATTCCCAGCACTTTGGGAGGTCAACCCCGGTGGATGACCTGAGGTCAGGA |
| | | | GTTCAAGACCAGCCTGGCCAACATGGTGAAACCCCATCTGTATAAAAATA |
| | | | CAAAAATTAGCTGGGCATGATAGCAGGTGCCTGTAATTCCAGCTACTCAG |
| | | | GAGGCTGAGGTGGGAGAA |
| SERPINE1 | rs2227679 | 8 | CAGAGATTCCCTCAGGGCCACATTTGTCCACCTCACTCCAGGGTCTCATC |
| | pos = 709 | | TTCGTGTGTTGCTAGTGTGATTACCTGGGGATGAGAAATCCTGCTGGGGG |
| | | | AGTTGAGGTTAAGAGGATGAGGACTCCAGGTGCTGTGGCTCACGCCTGTA |
| | | | ATCCCAGCACTTTGGGAGGCCAAGGCAGGTGGATCAGGAGTTTGAGGTCA |
| | | | GGAGTTTGAGACCAGCCTGGCCAACATGGTGAAACCCTGTCTCTACTAAA |
| | | | AATGCAAAAATTAGCCAGGTGTGGTGGCAGGCGCCTGTAATCCCAGCTAC |
| | | | TCGGGAGGCTGAGGCAGGAGAATCACTTGAGCCCGGGAGGTGGAGGTTGC |
| | | | AGTGAGCCGAACGAAATTGAGCCACTTCACCCCAGCCTGGGCAAAAGAGT |
| | | | GAAATTCCATTCAAAAAAAAAAAAAAAAAAAAAAGGATGAGGACTGGGAT |
| | | | GAACTGGTGGCTGGGTGTGGGGAAAATGGAAGTGAAGGAAGGCCAAAAGA |
| | | | GACAGAGAAGGCCTGGCGCGGCGACTCACGCCTATAATCCCAGCACTTTG |
| | | | GGAGGCTGAGAAGGGGGATTGCTTGAGGCCAGAAGTTGAATACCAGTCTG |
| | | | GGCAGCATAGCAAGACCCTGCCTCTACAAAAAAAAAATTTTTTTTAATTA |
| | | | GCCAGCCTTGGTGACATGCATCTGTAGTCTACTCAAGAAGCTGAGGTGAG |
| | | | GCCAGGCAYGGTGGCTCACGCCTGTATTCCCAGCACTTTGGGAGGTCAAG |
| | | | GCGGGTGGATGACCTGAGGTCAGGAGTTCAAGACCAGCCTGGCCAACATG |
| | | | GTGAAACCCCATCTGTATAAAAATACAAAAATTAGCTGGGCATGATAGCA |
| | | | GGTGCCTGTAATTCCAGCTACTCAGGAGGCTGAGGTGGGAGAATCTATTG |
| | | | AACCCGGGAGGGGGAGGTTGCAGTGAOCCGAGATCATGCCATTGCACTCC |
| | | | AGCCTGGGCGACAGAGTGAGACTCCTTCTCAAAACAAACAAACAAACAAA |
| | | | CAAACAAAATACAGAAGCTCAGGCGGGAGCAACATTTGAACCGGATTCGG |
| | | | AGGCTGCAGTGAGCTATGATTGCACCACTGCGCTCCAGTCTGTGTGACAG |
| | | | TGAGACCCTGTCTCTTACACACACACACACACACACACACATGCACACAC |
| | | | ACAGACAGAGAGAAATTAGAAGATACTGAATTGGCAGAAGAGAACGGAAA |
| | | | TAGAAATTAAAATACTGAATAGGGGAGCAGTGAACAGGGGATACCCAAAA |
| | | | GCCAAGAGCGAGAGAGAGCCTGGCTTCCAGAAATAGTGGAGAACCCAGGA |
| | | | GAACTAGGTGAAAACCCAGTGCTGGGTTGCCATCAGCGAGAGCTGGAGCC |
| | | | A |
| SERPINE1 | rs2227684 | 9 | CGGATTCGGAGGCTGCAGTGAGCTATGATTGCACCACTGCGCTCCAGTCT |
| | | | GTGTGACAGTGAGACCCTGTCTCTTACACACACACACACACACACACACA |
| | | | CACATGCACACACACAGAGAGAGAGAAATTAGAAGATACTGAATTGGCAG |
| | | | AAGAGAAGGGAAATAGAAATTAAAATACTGAATAGGGGAGCAGTGAACAG |
| | | | GGGATACCCAAAAGCCAAGAGCGAGAGAGAGCCTGGCTTCCAGAAATAGT |
| | | | GGAGAAGCCAGGAGAACTAGGTGAAAACCCAGTGCTGGGTTGCCATCAGC |
| | | | RAGAGCTGGAGCCATTTCCAACGAACCATCTTGTCGTCTTCACAGCTGAG |
| | | | TTCACCACGCCCGATGGCCATTACTACGACATCCTGGAACTGCCCTACCA |
| | | | CGGGGACACCCTCAGCATGTTCATTGCTCCCCCTTATGAAAAAGAGGTGC |
| | | | CTCTCTCTGCCCTCACCAACATTCTGAGTGCCCAGCTCATCAGCCACTGG |
| | | | AAAGGCAACATGACCAGGCTGCCCCGCCTCCTGGTTCTGCCCAAGTAAGC |
| | | | CACCCCGCTATCTCCCCGACCTACCAACCCCTCTCTCCTGGCTCCCTAAA |
| | | | G |
| SERPINE1 | 2227686 | 10 | CAGGCTGAATAAAATTATGCTGAAACTACTGTCTTATTTGAGGAAAGTAA |
| | | | TTAGTCATAGGTGGGAGGGGGTGGGGAGATTGCAGAAGAATGTTCATGAA |
| | | | TATTAGGATTTTCAGCTCTAAGGGGGGACTTTGTAAACAGCTTTACAAGA |
| | | | AGAACCAGGCCGGCTGGGTGTGGTGGCTCATGCCTGTAATCTCAGCATTT |
| | | | GGGGAGGCCAAGGCGGGCGGATCACTTGAGGTCAGGAGTTTGAGACCAGC |
| | | | CTGGCCAACATGGTGAAACCCTGTCTCTATTAAAAATACAAAAATTAGCC |
| | | | RGCCGTGGTAGCGAGCGCCTATGATCCCAGCTACTCCGGAGGCTGAGGCC |
| | | | AGAGAATCACATGAACCTGGGAGGTGGAGGCTGCAGTGAGCCGAGATCAC |
| | | | GCCACTGCACTCCAGCCTGGGGGACAGAGCAAGAATCTGTTTCAAAAAAA |
| | | | AAAAAAGAAAAATAGGAAGGAAGGAAGGAAAGGAAAGGAAAGAAGAGAGA |
| | | | GAGAAAGAAAGAGAGAGAAGAAAGAAAGAAAGAAAGAAAGAAAGAAAGAA |
| | | | AGAAAGAAAGAAAGAAACAAAGAAAAAGAAAGGAAAGAAAGAACGAACGA |
| | | | A |
| SERPINE1 | rs2227687 | 11 | GCTGAATAAAATTATGCTGAAACTACTGTCTTATTTGAGGAAAGTAATTA |
| | | | GTCATAGGTGGGAGGGGGTGGGGAGATTGCAGAAGAATGTTCATGAATAT |
| | | | TAGGATTTTCAGCTCTAAGGGGGGACTTTGTAAACAGCTTTACAAGAAGA |
| | | | ACCAGGCCGGCTGGGTGTGGTGGCTCATGCCTGTAATCTCAGCATTTGGG |
| | | | GAGGCCAAGGCGGGCGCATCACTTGAGGTCAGGAGTTTGAGACCAGCCTG |
| | | | GCCAACATGGTGAAACCCTGTCTCTATTAAAAATACAAAAATTAGCCAGC |
| | | | YGTGGTAGCGAGCGCCTATGATCCCAGCTACTCCGCAGGCTGAGGCCAGA |
| | | | GAATCACATGAACCTGGGAGGTGGAGGCTGCAGTGAGCCGAGATCACGCC |
| | | | ACTGCACTCCAGCCTGGGGGACAGAGCAAGAATCTGTTTCAAAAAAAAAA |
| | | | AAAGAAAAATAGGAAGGAAGGAAGGAAAGGAAAGGAAAGAAGAGAGAGAG |
| | | | AAAGAAAGAGAGAGAAGAAAGAAAGAAAGAAAGAAAGAAAGAAAGAAACA |
| | | | AAGAAAGAAAGAAAGAAAGAAAAAGAAAGGAAAGAAAGAACGAACGAACC |
| | | | A |
| SERPINE1 | rs2227703 | 12 | AAAAATGTTTCAAAAAAATAATAAAATAAATAAATACGAAGAATATGTCA |
| | pos = 256 | | GGACAGTCACTGCCTTCACCTTCTCCATTTCACACCGGTGGTACAAGAAA |
| | | | TCAGAAGCCTAGCCCAGGTGTGGTGGTTCATGCCTGTAATCCCAGCACTT |
| | | | TGGGAAGCCGAGGTGGGTGGATCACCTAAGGTCAGGAGTTTGAGACCAGC |
| | | | CTGGACAACATGGTGAAACCCCGTCTCTACTAAAAATACAAAAATTAGCC |
| | | | GGGCGYGGTGGCTGGCGCCTGTAATCCCAGCTACTCGGGAGGCTGAGGCA |
| | | | GGAGAATCACTTGAAGCCAGGAGGCAGAGGTTGCACTGAGCTGAGATTGC |
| | | | ACCACTGAACTCCAGGCTGGGTGGCAGAGCGAGACTCCCTCTCAAAAAAC |
| | | | AACAACTACAAAGACAACAACAAACCCAGAATCAAAATCCTGTTGGTCCA |
| | | | TAGACCTCATGGGTGGAAGAGACCTTCCTACATCCAGGTTGGCCCAACAT |
| | | | GGGGGAGTCCA |
| SERPINE1 | rs2227706 | 13 | ATAAACTTTCCACTTATGCAGATGGGCCTGCTCGTAAGTCACTGTCACTG |
| | pos = 201 | | TGGGTTCCCAACTCTCTTCATGACACTTCCTTCCAGCACCAAATGCTTCC |
| | | | CACCCCTCTACTCCCACTCCCCATTCTTCAAACCCAGCTCAAGTTCCAGT |
| | | | TCCTCCACCTAGGACTTCCCATGGATCCAGCCAATATCACTCTCAGGTCC |
| | | | RGCGCAGTGGCCCACGCCTGTAATCTCAGCACTTTGGGAGGCCGGGGCAG |
| | | | GAAGATTGCTTGAGGCCAGGAGTTTCAGACCAGCCTGGACAACATAGTGA |
| | | | GACTCTTCCTCTAAGAAAAGAAAGAGAGAAAGAGAGAGAGAAAGACAGAG |
| | | | GAAAGAGAGAGAAAAATAAAGAAAGAGAGAAAGAAACAAAGAAAAAGAAA |
| | | | AAGAAAGAAAGAACGAAAGAAAGAAAGAAAGAAAGAAAGAAAGAAAGAAA |
| | | | GAAAGAAAGAAAGAAAGAAAGAAAGAAAGAAAGAAAGAAAAAGAAAGGAA |
| | | | GGAAGGAAGGAAAGGAGGGAGGGAGGGAGGGAAGGAAGGAAGGAAGGAAG |
| | | | GAAGGGGATCAAAACATCATCACTCTCAACTCGACACTGACTGAGTTTTC |
| | | | TTCTCCCTGGTCTGTAACAGTGCTTGGATTCTCGAGTGTTCCTTAGCTTT |
| | | | GTGTGT |
| SERPINE1 | rs11560324 | 14 | AACAAGATCTGCCTCCAGCTACCGTTATTGTACATTTCTTGTAAACCTCA |
| | pos = 201 | | AACAGTGGAATTATTTATCTGCTCATCTGTGGGCAAAGAGTATGAATGTT |
| | | | GTAAATCCCTTAATGGTAAAAATATTCAGTCAAAAGCAAATTTGTCAAGG |
| | | | AGACACCATCTTTTTTTGAAATCATTTTTTTAGGTGGTTTCTCAAGTAAG |
| | | | STTTGTTGAAATAATAGTACCAAGCGGCTGGGCGTGGTGGCTCAGGCCTG |
| | | | TAATCCCAGCACTTTAGGAGGCCAAGGTGGGCGGATCACCTGAGGTCAGG |
| | | | AGTTTGAGACCAGCCTGGCCAACAAGGTGAAACCCTGTCTCTACTAAAAA |
| | | | TACAAAAATTAGGCCGGGTGTGGTGGCTCACACCTGTAATCCCAGCACTT |
| | | | TGGGAGGCCAAGGCGGGTGGATCACGAGGTCAGGAGTTCAAGACCAGCCT |
| | | | GGCCAGGTTGGTGAAACCCCGTCTCCACTAAAAACACACAAAAAATTAAC |
| | | | GAGGCGCAGTGGCGGGTGCCTGTAGTCCCAGCTACTCAGGAGGCTGAGGC |
| | | | AGGAGAATGGCGTGAAACCAGGAGGCAGAGCTTGCAACGAGCCGACATCA |
| | | | CGCCACTGGACTCCAGCCTGGGCAACAGAGTGAGACTCCGTCTCAAAAAA |
| | | | AAAAAAAAAAAAAAAAGTAGCAAGCACTCAGGAAAACTGAGACACAGAGA |
| | | | GGTTCAGTAACTTGATCTCATTATTATCATTATTTTAGAGAGGGGGTCTC |
| | | | TGTTGTTTAGGCTGGAGTGCAGTGGTGCAATCACAGCTCACTGCAGCCTT |
| | | | GAACTGCTGGGCTCAAACAATCCTGTCTCAGCCTCCCAAGCAGCTGGGAC |
| | | | TACAGGTGCGCACCACTCACTACCCAGCTAATTCGTTTTCTTTCTTTTTT |
| | | | TTTTTTTTTTAAAAAAAAGTCTCGCTCTGTCACCCATGCTGGAATGCAAT |
| | | | GGCTTGATCTCTCTGCTCACTACAACCTCCGCCTCCCGGGTTCAAGAGAT |
| | | | TCTCCTTTCTCAGCCTCCTGAGTAGCTGGGATTACAGGCATGTGCCACCA |
| | | | TGCCCGGTTAATTTTTGTATTTTTAGTAGAGATAGGGTTTTGCCATGTTG |
| | | | GCCAGGCTGGTCTCGAGCTTCTGACCTCAGGTAATCCACCTGCCTCAGCC |
| | | | TCCCAAAGTGCTGAGATTACAGGTGTGAGCCACTGCACCTGGCCCCAGCT |
| | | | AATTTTTTAATTGTATTTTTTGTAGACACAGGGTCTCACTATGTTGTCCA |
| | | | AGCTGGTCTCGAACTCCTGGGCTCAGCCTCCCAAAGTGCTGGGATTACAG |
| | | | GCGTGACCCACTGCACCCGGCCCCAGCTAATTTTTTAATTTTATTTTTTG |
| | | | TAGAGACAGGGTCTCACTATGTTGTTCAAGCTGGTCTTGAACTCTCGGGC |
| | | | TCAGCCTCCCAAAGTGCTGGCATTACAAGCATGAGCTACTGTGCCCAGCT |
| | | | GCTGCTATCATCAT |
| SERPINE1 | rs13238709 | 15 | AGTTCCAGTTCCTCCACCTAGGACTTCCCATGGATCCAGCCAATATCACT |
| | pos = 501 | | CTCAGGTCCGGCGCAGTGGCCCACGCCTGTAATCTCAGCACTTTGGGAGG |
| | | | CCGGGGCAGGAAGATTGCTTGACGCCAGGAGTTTCAGACCAGCCTGGACA |
| | | | ACATAGTGAGACTCTTCCTCTAAGAAAAGAAAGAGAGAAAGAGAGAGAGA |
| | | | AAGACAGAGGAAAGAGAGAGAAAAATAAAGAAAGAGAGAAAGAAAGAAAG |
| | | | AAAAAGAAAAAGAAAGAAAGAACGAAAGAAAGAAAGAAAGAAAGAAAGAA |
| | | | AGAAAGAAAGAAAGAAAGAAAGAAAGAAAGAAAGAAAGAAAGAAAGAAAA |
| | | | AGAAAGGAAGGAAGGAAGGAAAGGAGGGAGGGAGGGAGGGAAGGAAGGAA |
| | | | GGAAGGAAGGAAGGGGATCAAAACATCATCACTCTCAACTCGACACTGAC |
| | | | TGAGTTTTCTTCTCCCTGCTCTGTAACAGTGCTTGGATTCTCGAGTGTTC |
| | | | YTTAGCTTTGTGTGTGTGTGTCTTACCCTCCCCAAGCCCATCAAGGTATC |
| | | | AGGTTTCTTGAAAACAAGGCTCTCTTGTTATATATACTCTAGCATCTTCT |
| | | | TGAAAATGGCTTCCTGAAAAGTCTTTTATTATCTTTCTCCAGCATCTCGA |
| | | | AGGCTTTTGGCAGAGGGCACACTTCCCTCCATTAGTTTCTGTTCAAATAT |
| | | | T |
| PROC | rs971207 | 16 | CGTGCAGCGTCCTCCTCCATGTAGCCTGGCTGCGTTTTTCTCTGACGTTG |
| | pos = 201 | | TCCGGCGTGCATCGCATTTCCCTCTTTACCCCCTTGCTTCCTTGAGCAGA |
| | | | GAACAGAATCCCGATTCTGCCTTCTTCTATATTTTCCTTTTTATGCATTT |
| | | | TAATCAAATTTATATATGTATGAAACTTTAAAAATCAGAGTTTTACAACT |
| | | | YTTACATTTCAGCATGCTGTTCCTTGGCATGGGTCCTTTTTTCATTCATT |
| | | | TTCATTAAAAGGTGGACCCTTTTAATGTGGAAATTCCTATCTTCTGCCTC |
| | | | TAGGGACATTTATCACTTATTTCTTCTACAATCTCCCCTTTACTTCCTCT |
| | | | ATTTTCTCTTTCTGGACCTCCCATTATTCAGACCTCTTTCCTCTAGTTTT |
| | | | ATTGTCTCTTCTATTTCCCATCTCTTTGACTTTGTGTTTTCTTTCAGGGA |
| | | | ACTTTCTTTTTTTTCTTTTTTTTTGAGATGGAGTTTCACTCTTGTTGTCC |
| | | | CAGGCTGGAGTGCAATGACGTGATCTCAGCTCACCACAACCTCCGCCTCC |
| | | | TGGATTCAAGCGATTCTCCTGCCGCAGCCTCCCGAGTAGCTGGGATTACA |
| | | | GGCATGCGCCACCACCCCCACCTAATTTTGTGTTTTTAGTAGAGAAGGGG |
| | | | TTTCTCCGTGTTGGTCAAGCTGGTCTTGAACTCCTGACCTCAGGTGATCC |
| | | | ACCTGCCTTGGCCTCCTAAAGTGCTGGGATTACAGCCGTGAGCCACCGCG |
| | | | CCCAGCCTCTTTCAGGGAACTTTCTACAACTTTATAATTCAATTCTTCTG |
| | | | CAGAAAAAAATTTTTGGCCAGGCTCAGTAGCTCAGACCAATAATTCCAGC |
| | | | ACTTTGAGAGGCTGAGGTGGGAGGATTGCTTGAGCTTGGGAGTTTGAGAC |
| | | | TAGCCTGGGCAACACAGTGAGACCCTGTCTCTATTTTTAAAAAAAGTAAA |
| | | | AAAAGATCTAAAAATTTAACTTTTTATTTTGAAATAATTAGATATTTCCA |
| | | | GGAAGCTGCAAAGAAATGCCTGGTGGGCCTGTTGCCCTGTGGGTTTCCTG |
| | | | CAAGGCCTTGGGAAGGCCCTGTCATTGGCACAACCCCAGATCGTGAGGGC |
| | | | TTTCCTTTTAGGCTGCTTTCTAAGAGGACTCCTCCAAGCTCTTGGAGGAT |
| | | | GGAAGACGCTCACCCATGGTGTTCGGCCCC |
| PROC | rs973760 | 17 | AGCAGGCCTTGGCTGGCCTCTCTGATGGAGCAGGCATCAGGCACAGGCCG |
| | | | TGGGTCTCAACGTGGGCTGGGTGGTCCTGGACCAGCAGCAGCCGCCGCAG |
| | | | CAGCAACCCTGGTACCTGGTTAGGAACGCAGACCCTCTGCCCCCATCCTC |
| | | | CCAACTCTGAAAAACACTGGCTTAGGGAAAGGCGCGATGCTCAGGGGTCC |
| | | | CCCAAAGCCCGCAGGCAGAGGGAGTGATGGGACTGGAAGGAGGCCGAGTG |
| | | | ACTTGGTGAGGGATTCGGGTCCCTTGCATGCCAGAGGCTGCTGTGGGAGC |
| | | | RGACAGTCGCGAGAGCAGCACTGCAGCTGCATGGGGAGAGGGTGTTGCTC |
| | | | CAGGGACGTGGGATGGAGGCTGGGCGCGGGCGGGTGGCGCTGGAGGGCGG |
| | | | GGGAGGGGCAGGGAGCACCAGCTCCTAGCAGCCAACGACCATCGGGCGTC |
| | | | GATCCCTGTTTGTCTGGAAGCCCTCCCCTCCCCTGCCCGCTCACCCCCTG |
| | | | CCCTGCCCCACCCGGGCGCCCCCCCTCCGCACACCGGCTGCAGGAGCCTG |
| | | | ACGCTGCCCGCTCTCTCCGCAGCTGGCCTTCTGGTCCAAGCACGTCGGTG |
| | | | A |
| PROC | rs1518759 | 18 | CCCCCTTGCTTCCTTGAGGAGAGAACAGAATCCCGATTCTGCCTTCTTCT |
| | pos = 980 | | ATATTTTCCTTTTTATGCATTTTAATCAAATTTATATATGTATGAAACTT |
| | | | TAAAAATCAGAGTTTTACAACTTTTACATTTCAGCATGCTGTTCCTTGGC |
| | | | ATGGGTCCTTTTTTCATTCATTTTCATTAAAAGGTGGACCCTTTTAATGT |
| | | | GGAAATTCCTATCTTCTGCCTCTAGGGACATTTATCACTTATTTCTTCTA |
| | | | CAATCTCCCCTTTACTTCCTCTATTTTCTCTTTCTGGACCTCCCATTATT |
| | | | CAGACCTCTTTCCTCTAGTTTTATTGTCTCTTCTATTTCCCATCTCTTTG |
| | | | ACTTTGTGTTTTCTTTCAGGGAACTTTCTTTTTTTTCTTTTTTTTTGAGA |
| | | | TGGAGTTTCACTCTTGTTGTCCCAGGCTGGAGTGCAATGACGTGATCTCA |
| | | | GCTCACCACAACCTCCGCCTCCTGGATTCAAGCGATTCTCCTGCCGCAGC |
| | | | CTCCCGAGTAGCTGGGATTACAGGCATGCCCCACCACGCCCAGCTAATTT |
| | | | TGTGTTTTTAGTAGAGAAGGGGTTTCTCCGTGTTGGTCAAGCTCGTCTTG |
| | | | AACTCCTGACCTCAGGTGATCCACCTGCCTTGGCCTCCTAAAGTGCTGGG |
| | | | ATTACAGGCCTGAGCCACCGCGCCCAGCCTCTTTCAGGGAACTTTCTACA |
| | | | ACTTTATAATTCAATTCTTCTGCAGAAAAAAATTTTTGGCCAGGCTCAGT |
| | | | AGCTCAGACCAATAATTCCAGCACTTTGAGAGGCTGAGGTGGGAGGATTG |
| | | | CTTGAGCTTGGGAGTTTGAGACTAGCCTGGGCAACACAGTGAGACCCTGT |
| | | | CTCTATTTTTAAAAAAAGTAAAAAAACATCTAAAAATTTAACTTTTTATT |
| | | | TTGAAATAATTAGATATTTCCAGGAAGCTGCAAAGAAATGCCTGGTCGGC |
| | | | CTGTTGGCCTGTGGGTTTCCTGCAACGCCKTGGGAAGGCCCTGTCATTGG |
| | | | CAGAACCCCAGATCGTGAGGGCTTTCCTTTTAGGCTGCTTTCTAAGAGGA |
| | | | CTCCTCCAACCTCTTGGAGGATGGAAGACGCTCACCCATGGTGTTCGGCC |
| | | | CCTCAGAGCAGGGTGGGGCAGGGGAGCTGGTGCCTGTGCAGGCTGTGGAC |
| | | | ATTTGCATGACTCCCTGTGGTCAGCTAAGA |
| PROC | rs2069913 | 19 | TGCACACTGGCCTCACGGCTGCCCTGCCCCAACCCCTTTCCTGGTCTCCA |
| | | | CAGCCAACGGGAGGAGGCCATGATTCTTGGGGAGGTCCGCAGGACACATG |
| | | | GGCCCCTAAAGCCACACCAGGCTGTTGGTTTCATTTCTGCCTTTATAGAG |
| | | | CTGTTTATCTGCTTGGGACCTGCACCTCCACCCTTTCCCAAGGTGCCCTC |
| | | | AGCTCAGGCATACCCTCCTCTAGGATGCCTTTTCCCCCATCCCTTCTTGC |
| | | | TCACACCCCCAACTTGATCTCTCCCTCCTAACTGTGCCCTGCACCCAACA |
| | | | SAGACACTTCACAGAGCCCAGGAGACACCTGGGGACCCTTCCTGGGTGAT |
| | | | AGGTCTGTCTATCCTCCAGGTGTCCCTGCCCAAGGGGAGAAGCATGGGGA |
| | | | ATACTTGGTTGGGGGAGGAGAGGAAGACTCGGGGGATGTGTCAAGATGGG |
| | | | GCTGCACGTGGTGTACTGGCAGAAGAGTGAGAGGATTTAACTTGGCAGCC |
| | | | TTTACAGCAGCAGCCAGGGCTTGAGTACTTATCTCTGGGCCAGGGACTGT |
| | | | ATTGGATGTTTTACATGACGGTCTCATCCCCATGTTTTTGGATGAGTAAA |
| | | | T |
| PROC | rs2069914 | 20 | CACGGCTGCCCTGCCCCAACCCCTTTCCTGGTCTCCACAGCCAACGGGAG |
| | | | GAGGCCATCATTCTTGGGGAGGTCCGCAGGACACATGGGCCCCTAAAGCC |
| | | | ACACCAGGCTGTTGGTTTCATTTGTGCCTTTATACAGCTGTTTATCTGCT |
| | | | TGGGACCTGCACCTCCACCCTTTCCCAAGGTGCCCTCAGCTCAGGCATAC |
| | | | CCTCCTCTAGGATGCCTTTTCCCCCATCCCTTCTTGCTCACACCCCCAAC |
| | | | TTGATCTCTCCCTCCTAACTGTGCCCTGCACCCAAGACAGACACTTCACA |
| | | | RAGCCCAGGAGACACCTCGGGACCCPTCCTGGGTCATAGGTCTGTCTATC |
| | | | CTCCAGGTGTCCCTGCCCAAGGGGAGAAGCATGGGGAATACTTGGTTGGG |
| | | | GGACGACAGGAAGACTGGGGGGATGTCTCAAGATGGGGCTCCACCTGGTG |
| | | | TACTGGCAGAAGACTGAGAGGATTTAACTTGGCACCCTTTACAGCAGCAG |
| | | | CCAGGGCTTGAGTACTTATCTCTGGGCCAGGGACTGTATTGGATGTTTTA |
| | | | CATGACCGTCTCATCCCCATGTTTTTCGATGAGTAAATTGAACCTTAGAA |
| | | | A |
| PROC | rs2069918 | 21 | GTAAGGCCACCATGGGTCCAGAGGATGAGGCTCAGGGGCGAGCTGGTAAC |
| | | | CAGCAGGGGCCTCGAGGAGCAGGTGGGGACTCAATGCTGAGGCCCTCTTA |
| | | | GGAGTTGTGGGGGTGGCTGAGTGGAGCGATTAGGATGCTGGCCCTATGAT |
| | | | GTCGGCCACGCACATGTGACTGCAAGAAACAGAATTCAGGAAGAAGCTCC |
| | | | AGGAAAGAGTGTGGGGTGACCCTAGGTGCGGACTCCCACCAGCCACAGTG |
| | | | TAGGTGGTTCAGTCCACCCTCCAGCCACTGCTGAGCACCACTGCCTCCCC |
| | | | RTCCCACCTCACAAAGAGGGGACCTAAAGACCACCCTGCTTCCACCCATG |
| | | | CCTCTGCTGATCAGGGTGTGTGTGTGACCGAAACTCACTTCTGTCCACAT |
| | | | AAAATCGCTCACTCTGTGCCTCACATCAAAGGGACAAAATCTGATTGTTC |
| | | | AGGGGGTCGGAAGACAGGGTCTGTGTCCTATTTGTCTAAGGGTCAGAGTC |
| | | | CTTTGGAGCCCCCAGAGTCCTGTGGACGTGGCCCTAGGTAGTAGGGTGAG |
| | | | CTTGGTAACGGGGCTGGCTTCCTGAGACAAGGCTCAGACCCGCTCTGTCC |
| | | | C |
| PROC | rs2069921 | 22 | CAGAGTCCTGTGGACGTGGCCCTAGGTAGTAGGGTGAGCTTGGTAACGGG |
| | | | GCTGGCTTCCTGAGACAAGGCTCAGACCCGCTCTGTCCCTGGGGATCGCT |
| | | | TCAGCCACTAGGACCTGAAAATTGTGCACGGCCTGGGCCCCCTTCCAAGG |
| | | | CATCCAGGGATGCTTTCCAGTGGAGGCTTTCAGGGCAGGAGACCCTCTGG |
| | | | CCTGCACCCTCTCTTGCCCTCAGCCTCCACCTCCTTGACTGGACCCCCAT |
| | | | CTGGACCTCCATCCCCACCACCTCTTTCCCCAGTGGCCTCCCTGGCAGAC |
| | | | RCCACAGTGACTTTCTGCAGGCACATATCTCATCACATCAAGTCCCCACC |
| | | | GTGCTCCCACCTCACCCATGGTCTCTCAGCCCCAGCAGGCCTTGGCTGGC |
| | | | CTCTCTGATGGAGCAGGCATCAGGCACAGGCCGTGGGTCTCAACGTGGGC |
| | | | TGGGTGGTCCTGGACCAGCAGCAGCCGCCGCAGCAGCAACCCTGGTACCT |
| | | | GGTTAGGAACGCAGACCCTCTGCCCCCATCCTCCCAACTCTGAAAAACAC |
| | | | TGGCTTAGGGAAAGGCGCGATGCTCAGGGGTCCCCCAAAGCCCGCAGGCA |
| | | | G |
| PROC | rs2069933 | 23 | CCCCCGTGGGGCTTGGCTTAGAATTCCCAGGTGCTCTTCCCAGGGAACCA |
| | | | TCAGTCTGGACTGAGAGGACCTTCTCTCTCAGGTGGGACCCGCCCCTGTC |
| | | | CTCCCTGGCAGTGCCGTGTTCTGGGGGTCCTCCTCTCTGGGTCTCACTGC |
| | | | CCCTGGGGTCTCTCCAGCTACCTTTGCTCCACGTTCCTTTGTGGCTCTGG |
| | | | TCTGTGTCTGGGGTTTCCAGGGGTCTCGGGCTTCCCTGCTGCCCATTCCT |
| | | | TCTCTGGTCTCACGGCTCCGTGACTCCTGAAAACCAACCAGCATCCTACC |
| | | | YCTTTGGGATTGACACCTGTTGGCCACTCCTTCTGGCAGGAAAAGTCACC |
| | | | GTTGATAGGGTTCCACGGCATAGACAGGTGGCTCCGCGCCAGTGCCTGGG |
| | | | ACGTGTGGGTGCACAGTCTCCGGGTGAACCTTCTTCAGGCCCTCTGCCCA |
| | | | GGCCTGCAGGGGCACAGCAGTGGGTGGGCCTCAGGAAAGTGCCACTGGGG |
| | | | AGAGGCTCCCCGCAGCCCACTCTGACTGTGCCCTCTGCCCTGCAGGAGAG |
| | | | TATGACCTGCGGCGCTGGGAGAAGTGGGAGCTGGACCTGGACATCAAGGA |
| | | | G |

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the SNP of interest.

A “genotype” is defined as the genetic constitution of an organism, usually in respect to one gene or a few genes or a region of a gene relevant to a particular context (i.e. the genetic loci responsible for a particular phenotype).

A “phenotype” is defined as the observable characters of an organism.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins” or “I”) of a nucleotide relative to a reference allele. Furthermore, a person of skill in the art would appreciate that an insertion or deletion within a given sequence could alter the relative position and therefore the position number of another polymorphism within the sequence. Furthermore, although an insertion or deletion may by some definitions not qualify as a SNP as it may involve the deletion of or insertion of more than a single nucleotide at a given position, as used herein such polymorphisms are also called SNPs as they generally result from an insertion or deletion at a single site within a given sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature >38° C. or <36° C., B) heart rate >90 beats per minute, C) respiratory rate >20 breaths per minute or the need for mechanical ventilation, and D) white blood cell count >12,000 per mm³ or <4,000 mm³. In the following description, the presence of two, three, or four of the “SIRS” criteria were scored each day over the 28 day observation period.

“Sepsis” is defined as the presence of at least two “SIRS” criteria and known or suspected source of infection. Severe sepsis is defined as sepsis plus one new organ failure by Brussels criteria or by the definition described in the PROWESS study (BERNARD G R et al. (2001) N Engl J Med 344(10):699-709).

Subject outcome or prognosis as used herein refers the ability of a subject to recover from an inflammatory condition and may be used to determine the efficacy of a treatment regimen, for example the administration of XIGRIS™. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELLP syndrome, mycobacterial tuberculosis, Pneumocystis carinii pneumonia, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis, disseminated intravascular coagulation (DIC), cardiogenic shock, and acute kidney injury.

Assessing subject outcome or prognosis may be accomplished by various methods. For Example, an “APACHE II” score is defined as Acute Physiology And Chronic Health Evaluation and herein was calculated on a daily basis from raw clinical and laboratory variables. Vincent et al. (VINCENT J L. FERREIRA F. MORENO R. Scoring systems for assessing organ dysfunction and survival. Critical Care Clinics. 16:353-366, 2000) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.” Furthermore, the criteria or indication for administering activated protein C (XIGRIS™-drotrecogin alfa (activated)) in the United States is an APACHE II score of ≧25. In Europe, the criteria or indication for administering activated protein C is an APACHE II score of ≧25 or 2 organ system failures.

“Protein C” or “protein C like compound” as used herein includes any protein C molecule, protein C derivative, protein C variant, protein C analog and any prodrug thereof, metabolite thereof, isomer thereof, combination(s) of isomers thereof, or pharmaceutical composition of any of the preceding including pharmaceutically acceptable salts thereof, wherein the “protein C” or “protein C like compound” has anti-inflammatory agent or the anti-coagulant activity in a subject. Protein C or protein C like compound(s) may be synthesized or purified. For example, Drotrecogin alfa (activated) is sold as XIGRIS™ by Eli Lilly and Company and has the same amino acid sequence as human plasma-derived Activated Protein C. Examples of derivatives, variants, analogs, or compositions etc. may be found in US patent applications: 20050176083; 20050143283; 20050095668; 20050059132; 20040028670; 20030207435; 20030027299; 20030022354; and 20030018175 and issued U.S. Pat. Nos. 6,933,367; 6,841,371; 6,815,533; 6,630,138; 6,630,137; 6,436,397; 6,395,270; 6,162,629; 6,159,468; 5,837,843; 5,453,373; 5,330,907; 5,766,921; 5,753,224; 5,516,650; and 5,358,932.

“Activated protein C” is also known as Drotrecogin alfa (activated) and is sold as XIGRIS™ by Eli Lilly and Company. Drotrecogin alfa (activated) is a serine protease glycoprotein of approximately 55 kilodalton molecular weight and having the same amino acid sequence as human plasma-derived Activated Protein C. The protein consists of a heavy chain and a light chain linked by a disulfide bond. XIGRIS™, Drotrecogin alfa (activated) is indicated for the reduction of mortality in adult subjects with severe sepsis (sepsis associated with acute organ dysfunction) who have a high risk of death (e.g., as determined by an APACHE II score of ≧25 or having 2 or more organ system failures).

XIGRIS™ is available in 5 mg and 20 mg single-use vials containing sterile, preservative-free, lyophilized drug. The vials contain 5.3 mg and 20.8 mg of drotrecogin alfa (activated), respectively. The 5 and 20 mg vials of XIGRIS™ also contain 40.3 and 158.1 mg of sodium chloride, 10.9 and 42.9 mg of sodium citrate, and 31.8 and 124.9 mg of sucrose, respectively.

XIGRIS™ is currently recommended for intravenous administration at an infusion rate of 24 mcg/kg/hr for a total duration of infusion of 96 hours. Dose adjustment based on clinical or laboratory parameters is currently not recommended. If the infusion is interrupted, it is currently recommended that when restarted the infusion rate should be 24 mcg/kg/hr. Dose escalation or bolus doses of drotrecogin alfa are currently not recommended. However, recommendations for the use of drotrecogin alfa may change and current recommendations are not intended to limit the present description of drotrecogin alfa. XIGRIS™ may be reconstituted with Sterile Water for Injection and further diluted with sterile normal saline injection. These solutions must be handled so as to minimize agitation of the solution (Product information. XIGRIS™, Drotrecogin alfa (activated), Eli Lilly and Company, November 2001).

Drotrecogin alfa (activated) is a recombinant form of human Activated Protein C, which may be produced using a human cell line expressing the complementary DNA for the inactive human Protein C zymogen, whereby the cells secrete protein into the fermentation medium. The protein may be enzymatically activated by cleavage with thrombin and subsequently purified. Methods, DNA compounds and vectors for producing recombinant activated human protein C are described in U.S. Pat. Nos. 4,775,624; 4,992,373; 5,196,322; 5,270,040; 5,270,178; 5,550,036; 5,618,714 all of which are incorporated herein by reference.

Treatment of sepsis using activated protein C in combination(s) with a bactericidal and endotoxin neutralizing agent is described in U.S. Pat. No. 6,436,397; methods for processing protein C is described in U.S. Pat. No. 6,162,629; protein C derivatives are described in U.S. Pat. Nos. 5,453,373 and 6,630,138; glycosylation mutants are described in U.S. Pat. No. 5,460,953; and Protein C formulations are described in U.S. Pat. Nos. 6,630,137, 6,436,397, 6,395,270 and 6,159,468, all of which are incorporated herein by reference.

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as described below. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO₂/FiO₂ ratio is less than 300 mmHg, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28-day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine or phenylephrine). Days alive and free of an International Normalized Ratio (INR) >1.5 are days that a person is alive and does not have an INR >1.5.

TABLE 2A
Brussels Organ Dysfunction Scoring System

| | ORGANS | | | | |
|---|---|---|---|---|---|
| | Free of Organ | | | | |
| | Dysfunction | | Clinically Significant Organ Dysfunction | | |
| | Normal | Mild | Moderate | Severe | Extreme |
| | DAF ORGAN DYSFUNCTION SCORE | | | | |
| | 1 | | 0 | | |
| Cardiovascular | >90 | ≦90 | ≦90 | ≦90 plus | ≦90 plus |
| Systolic BP | | Responsive to | Unresponsive to | pH ≦7.3 | pH ≦7.2 |
| (mmHg) | | fluid | fluid | | |
| Pulmonary | >400 | 400-301 | 300-201 | 200-101 | ≦100 |
| Pₐo₂/F_{I}o₂ (mmHg) | | | Acute lung injury | ARDS | Severe ARDS |
| Renal | <1.5 | 1.5-1.9 | 2.0-3.4 | 3.5-4.9 | ≧5.0 |
| Creatinine (mg/dL) | | | | | |
| Hepatic | <1.2 | 1.2-1.9 | 2.0-5.9 | 6.0-11.9 | ≧12 |
| Bilirubin (mg/dL) | | | | | |
| Hematologic | >120 | 120-81 | 80-51 | 50-21 | ≦20 |
| Platelets (×10⁵/mm³) | | | | | |
| Neurologic | 15 | 14-13 | 12-10 | 9-6 | ≦5 |
| (Glasgow Coma | | | | | |
| Score) | | | | | |

Round Table Conference on Clinical Trials for the Treatment of Sepsis Brussels, Mar. 12-14, 1994.

A clinical trial “adverse event” (AE) is defined as any undesirable experience, unanticipated benefit, or pregnancy that occurs after informed consent for the study has been obtained, without regard to the possibility of a causal relationship and without regard to treatment group assignment, even if no study drug has been taken.

A clinical trial “serious adverse event” (SAE) is defined as any untoward medical occurrence that was not a clinical outcome or was a clinical outcome but was believed by the investigator to be causally related to study drug infusion and resulted in any of the following: 1. Was life-threatening (Note: A life threatening event is one in which the patient was at risk of death at the time of the event. It does not refer to an event, which hypothetically might have caused death, if it were more severe.). 2. Required inpatient hospitalization or prolongation of existing Hospitalization. 3. Resulted in persistent or significant disability/incapacity. 4. Resulted in a congenital anomaly/birth defect. 5. Resulted in cancer. 6. Did not meet any of the serious criteria, but suggested a significant hazard, contraindication, side effect, or precaution as determined by the investigator.

We have found that genotyping a subject for a combination(s) of at least one SERPINE1 SNP and at least one PROC SNP is particularly useful in the prognostic classification of a subject for outcome from an inflammatory condition, and for responsiveness to treatment of the inflammatory condition with an anti-inflammatory or anti-coagulant agent. Genotyping may be determined for either the haploid genotype or diploid genotype, usually the diploid genotype. SNPs of interest include the specific SNPs of SERPINE1 and PROC described herein, as well as SNPs in linkage disequilibrium with the SNPs markers described herein,

In one embodiment, a subject sample e.g. a nucleic acid sample, is genotyped for (A) at least one SERPINE1 SNP or polymorphic site in linkage disequilibrium thereto, and (B) at least one PROC SNP or polymorphic site in linkage disequilibrium thereto. Specifically such genotypic combinations are shown herein to prognostically classify a patient as having increased (or decreased) likelihood of recovering from an inflammatory condition e.g. an inflammatory condition associated with bacterial infection. Specifically, such genotype combinations are also shown herein to prognostically classify a patient as having increased (or decreased) likelihood of responsiveness to the treatment of the inflammatory condition with an anti-inflammatory agent or an anti-coagulant agent. As mentioned above, these genotypic combinations are referred to as “improved response genotype combination(s),” IRGC and “non-response genotype combinations,” NRCG. A “mixed response genotype combination(s)” MRGC may be a genotype with a responsive allele from either SERPINE1 or PROC, but not both.

In accordance with a further embodiment, methods are provided for prognostic classification of a subject having an inflammatory condition according to the ability of the subject to respond to treatment of the inflammatory condition with an anti-inflammatory agent or an anti-coagulant agent, the method may include determining the genotype or genotyping the subject for at least one SERPINE1 SNP and at least one PROC SNP, or one or more polymorphic sites in linkage disequilibrium thereto; wherein the genotype thus obtained is indicative of the subject's ability to respond to treatment of the inflammatory condition with the anti-inflammatory agent or anti-coagulant agent.

Furthermore, methods are provided for prognostic classification of a subject having an inflammatory condition according to the ability of the subject to recover from the inflammatory condition, the method may include determining the genotype or genotyping the subject for at least one SERPINE1 SNP and at least one PROC SNP, or one or more polymorphic sites in linkage disequilibrium thereto; wherein the genotype thus obtained may be indicative of the subject's ability to respond to treatment of the inflammatory condition with the anti-inflammatory agent or anti-coagulant agent.

In some embodiments, the SERPINE1 SNP is rs7242; or a polymorphic site in linkage disequilibrium thereto, including rs11178; rs757716; rs2070682; rs2227662; rs2227673; rs2227679; rs2227684; rs2227686; rs2227687; rs2227703; rs2227706; rs11560324; and rs13238709.

In some embodiments, the PROC SNP is rs2069912; or a polymorphic site in linkage disequilibrium thereto, including rs971207; rs973760; rs1518759; rs2069913; rs2069914; rs2069918; rs2069921 and rs2069933.

The methods may further include classifying the subject as having an improved response genotype combination (IRGC), non-response genotype combination (NRGC) or a mixed response genotype combination (MRGC) as described herein.

A subject classified as having a NRGC may be further classified as having an increased risk of having an adverse event or a serious adverse event after treatment of the inflammatory condition with an anti-inflammatory agent or an anti-coagulant agent.

In some embodiments, genotyping may be performed by contacting a subject sample with two or more oligonucleotides selected from group consisting of: (I) an oligonucleotide that specifically hybridizes to a SERPINE1 SNP; and (II) an oligonucleotide that specifically hybridizes to a PROC SNP. Genotyping may further be performed by contacting a subject sample with at least two oligonucleotides that hybridize to each said SNP, wherein for each SNP a first oligonucleotide specifically hybridizes to one polymorphic variant at that SNP and a second oligonucleotide specifically hybridizes to another polymorphic variant at that SNP.

Oligonucleotide or oligonucleotides that specifically hybridize(s) to a SERPINE1 SNP include those that specifically hybridize to a polymorphic variants of SEQ ID NO:1; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; or SEQ ID NO:15. Oligonucleotide or oligonucleotides that specifically hybridize(s) to a PROC SNP include those that specifically hybridizes to a polymorphic variants of SEQ ID NO:2; SEQ ID NO:16; SEQ ID NO:17; SEQ ID NO:18; SEQ ID NO:19; SEQ ID NO:20; SEQ ID NO:21; SEQ ID NO:22 or SEQ ID NO:23.

The methods may further include: (a) selective administration to a subject of an anti-inflammatory agent or an anti-coagulant agent; wherein the subject has been classified as having one or more IRGC; (b) selective administration of an anti-inflammatory agent or an anti-coagulant agent to a subject; wherein the subject has been classified as having an IRGC or a MRGC; and (c) selectively not administering an anti-inflammatory agent or an anti-coagulant agent to a subject; wherein the subject has been classified as having a NRGC.

In some embodiments, the anti-inflammatory and/or anti-coagulant agent includes protein C, a protein-C like compound, an activated protein C, or drotecogin alfa (activated). The inflammatory conditions wherein the methods may be applied can be selected from SIRS; sepsis, severe sepsis; and septic shock.

The methods may further include determining the subject's APACHE II score as an assessment of subject risk, wherein the subject's APACHE II score is indicative of an increased risk when ≧25. The methods may further include determining the number of organ system failures for the subject as an assessment of subject risk, wherein 2 or more organ system failures are indicative of increased subject risk. The method may further include taking the subject's APACHE II score and/or the subject's number of organ failures into account in determining whether to selectively administer an anti-inflammatory agent or an anti-coagulant agent. The methods may further include measuring the level or concentration of PROC and/or PAI-1 proteins. The methods may further include determining the ratio of PAI-1/PROC protein levels in a sample from a subject, e.g. serum or plasma.

The two or more oligonucleotides or analogs thereof e.g. peptide nucleic acids, LNA, etc. may be selected from the group consisting of: (I) an oligonucleotide or analog thereof that specifically hybridizes to a SERPINE1 SNP; and (II) an oligonucleotide or analog thereof that specifically hybridizes to a PROC SNP. In some embodiments, the oligonucleotide of Group I specifically hybridizes to one of the provided polymorphic variants of SEQ ID NO:1; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; or SEQ ID NO:15. In some embodiments of the invention, the oligonucleotide of Group II specifically hybridizes to one of the provided polymorphic variants of SEQ ID NO:2; SEQ ID NO:16; SEQ ID NO:17; SEQ ID NO:18; SEQ ID NO:19; SEQ ID NO:20; SEQ ID NO:21; SEQ ID NO:22 or SEQ ID NO:23.

In one embodiment, illustrated herein, samples from subjects having an inflammatory condition, such as severe sepsis, were genotyped for SERPINE1 rs7242 and PROC 2069912. Some of the subjects were treated with activated Protein C (XIGRIS™), and some served as control subjects.

As further described in detail in Examples 3 and 4, broadly speaking, SERPINE1 rs7242/PROC rs2069912—IRGC subjects showed an increased likelihood of responding well to, and benefiting from, activated Protein C XIGRIS™, whereas SERPINE1 rs7242/PROC rs2069912—NRGC subjects did not. SERPINE1 rs7242/PROC rs2069912—MRGC subjects had an intermediate response. As further shown, SERPINE1 rs7242/PROC rs2069912-NRGC subjects had an increased likelihood of having a serious adverse event following the administration of activated Protein C compared to SERPINE1 rs7242/PROC rs2069912-IRGC and SERPINE1 rs7242/PROC rs2069912—MRGC subjects.

It will be appreciated that such genotype combinations may be useful for prognostically classifying subjects according to their ability to respond to an anti-inflammatory agent or an anti-coagulant agent, as well as their likelihood of having a severe adverse event following the administration of an anti-inflammatory agent or an anti-coagulant agent.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulonephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, cirrhosis, disseminated intravascular coagulation (DIC), cardiogenic shock, and acute kidney injury.

Once a subject is identified as being at risk for developing or having an inflammatory condition or is to be administered XIGRIS™, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material, particularly, genetic material containing the sequence or sequences of interest. Many methods are known in the art for collecting biological samples and extracting genetic material from those samples. Genetic material can be extracted from blood, tissue, hair and other biological material. There are many methods known to isolate DNA and RNA from biological material. Typically, DNA may be isolated from a biological sample when first the sample is lysed and then the DNA is separated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR), Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of a mutation in a SERPINE1/PROC gene sequence, as described in TABLES 1A-D. The sequence of interest may also include other mutations, or may also contain some of the sequence surrounding the mutation of interest.

Detection or determination of a nucleotide identity, or the presence of one or more single nucleotide polymorphism(s) (SNP typing), may be accomplished by any one of a number methods or assays known in the art. Many DNA typing methodologies are useful for use in the detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

Alternatively, an invasive cleavage method requires an oligonucleotide called an Invader™ probe and sequence-specific probes to anneal to the target DNA with an overlap of one nucleotide. When the sequence-specific probe is complementary to the polymorphic base, overlaps of the 3′ end of the invader oligonucleotide form a structure that is recognized and cleaved by a Flap endonuclease releasing the 5′ arm of the allele specific probe. 5′ exonuclease activity or TaqMann™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

The Illumina Golden Gate™ Assay uses a combined oligonucleotide ligation assay/allele-specific hybridization approach (SHEN R et al Mutat Res 2005573:70-82). The first series of steps involve the hybridization of three oligonucleotides to a set of specific target SNPs; two of these are fluorescently-labelled allele-specific oligonucleotides (ASOs) and the third a locus-specific oligonucleotide (LSO) binding 1-20 by downstream of the ASOs. A second series of steps involve the use of a stringent polymerase with high 3′ specificity that extends only oligonucleotides specifically matching an allele at a target SNP. The polymerase extends until it reaches the LSO. Locus-specificity is ensured by requiring the hybridization of both the ASO and LSO in order that extension can proceed. After PCR amplification with universal primers, these allele-specific oligonucleotide extension products are hybridized to an array which has 1536 discretely tagged addresses which match an address embedded in each LSO. Fluorescent signals produced by each hybridization product are detected by a bead array reader from which genotypes at each SNP locus are ascertained.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP system.

Mutation detection methods may include but are not limited to the following:

Restriction Fragment Length Polymorphism (RFLP) strategy—An RFLP gel-based analysis can be used to indicate the presence or absence of a specific mutation at polymorphic sites within a gene. Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. Finally, if both the higher molecular weight band and the two lower molecular weight bands are visible then the DNA sample contained both polymorphisms, and therefore the DNA sample, and by extension the subject providing the DNA sample, was heterozygous for this polymorphism;

For example the Maxam-Gilbert technique for sequencing (MAXAM A M. and GILBERT W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et al. (Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Q13 replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4):1760-1764). Other methods include those of Donis-Keller et al. (1977, Nucl. Acids Res. 4:2527-2538), SIMONCSITS A. et al. (Nature (1977) 269(5631):833-836), AXELROD V D. et al. (Nucl. Acids Res. (1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-terminator incorporation with fluorescent polarization-detection (TDI-FP) method is described by FREEMAN B D. et al. (J Mol Diagnostics (2002) 4(4):209-215) for large scale screening; Oligonucleotide ligation assay (OLA) is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2):123-9; IANNONE M A. et al. Cytometry (2000) 39(2):131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8.);

Polymerase proofreading methods are used to determine SNPs identities, as described in WO 0181631;

Detection of single base pair DNA mutations by enzyme-amplified electronic transduction is described in PATOLSKY F et al. Nat Biotech. (2001) 19(3):253-257;

Gene chip technologies are also known for single nucleotide polymorphism discrimination whereby numerous polymorphisms may be tested for simultaneously on a single array (EP 1120646 and GILLES P N. et al. Nat. Biotechnology (1999) 17(4):365-70);

Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43:1151-1158; LITTLE D P. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553).

Alternatively, a Single-Stranded Conformational Polymorphism (SSCP) assay or a Cleavase Fragment Length Polymorphism (CFLP) assay may be used to detect mutations as described herein.

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites.

Once the identity of a polymorphism(s) is determined or detected an indication may be obtained as to subject response to XIGRIS™ administration based on the genotype (the nucleotide at the position) of the polymorphism of interest. In the present invention, polymorphisms in SERPINE1/PROC gene sequences, are used to predict a subject's response to XIGRIS™ treatment. Methods for predicting a subject's response to XIGRIS™ treatment may be useful in making decisions regarding the administration of XIGRIS™.

Methods of treatment of an inflammatory condition in a subject having an improved response genotype in a SERPINE1/PROC gene are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, central nervous system, coagulation [INR >1.5], renal and/or hepatic).

As described above genetic sequence information or genotype information may be obtained from a subject wherein the sequence information contains one or more polymorphic sites in a SERPINE1/PROC gene sequence. Also, as previously described the sequence identity of one or more polymorphisms in a SERPINE1/PROC gene sequence of one or more subjects may then be detected or determined. Furthermore, subject response to administration of XIGRIS™ may be assessed as described above. For example, the APACHE II scoring system or the Brussels or SOFA scores may be used to assess a subject's response to treatment by comparing subject scores before and after treatment. Once subject response has been assessed, subject response may be correlated with the sequence identity of one or more polymorphism(s). The correlation of subject response may further include statistical analysis of subject outcome scores and polymorphism(s) for a number of subjects.

Methods of treatment of an inflammatory condition in a subject having one or more of the risk genotypes in SERPINE1 and PROC (or a SNP in linkage disequilibrium thereto) associated with improved response to a therapeutic agent are described herein. An improved response may include an improvement subsequent to administration of said therapeutic agent, whereby the subject has an increased likelihood of survival, reduced likelihood of organ damage or organ dysfunction (Brussels or SOFA scores), an improved APACHE II score, days alive and free of pressors, inotropes, and reduced systemic dysfunction (cardiovascular, respiratory, ventilation, central nervous system, coagulation [INR >1.5], renal and/or hepatic).

As described above genetic sequence information or genotype information may be obtained from a subject wherein the sequence information contains one or more single nucleotide polymorphic sites in SERPINE1 and PROC sequences. Also, as previously described the sequence identity of one or more single nucleotide polymorphisms in the SERPINE1 and PROC sequences of one or more subjects may then be detected or determined. Furthermore, subject outcome or prognosis may be assessed as described above, for example the APACHE II scoring system or the Brussels or SOFA scores may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis of subject outcome scores and polymorphism(s) for a number of subjects.

### 3. Analytical Methods

a. St. Paul's Hospital (SPH) Severe Sepsis Cohort

### Patient Cohort Selection

All subjects admitted to the ICU of St. Paul's Hospital (SPH) with SIRS were screened for study inclusion. SPH ICU is a mixed medical-surgical ICU in a tertiary care, university-affiliated teaching hospital. Subjects were included in the study if they met at least two out of four SIRS criteria: 1) fever (>38° C.) or hypothermia (<36° C.), 2) tachycardia (>90 beats/minute), 3) tachypnea (>20 breaths/minute), PaCO2 <32 mm Hg, or need for mechanical ventilation, and 4) leukocytosis (total leukocyte count >12,000 mm³) or leukopenia (<4,000 mm³). Subjects were included in the analysis if they met the diagnostic criteria for severe sepsis (SIRS criteria due to infection plus one new organ failure) on admission to the ICU. Subjects were excluded if blood could not be obtained for genotype analysis. Baseline characteristics (age, gender, admission APACHE II score (KNAUS W A. et al. Crit. Care Med. (1985) 13:818-829), together with medical vs. surgical diagnosis KNAUS W A. et al. Chest (1991) 100:1619-1636.) were recorded on admission to the ICU. The full cohort meeting these criteria included 1072 Caucasian subjects and 153 Asian subjects. XIGRIS™-treated subjects are defined as critically ill patients with severe sepsis, no XIGRIS™ contraindications and treated with XIGRIS™. Control subjects are critically ill patients who had severe sepsis (i.e. at least 2 of 4 SIRS criteria, known or suspected infection, and APACHE II ≧25), a platelet count >30,000/mm³, INR <3.0, bilirubin <20 mmol/L (i.e. no evidence of chronic hepatic dysfunction) and were not treated with XIGRIS™. Accordingly, the control group (i.e., untreated with XIGRIS™) is comparable to the XIGRIS™-treated group.

The Institutional Review Board at Providence Health Care and the University of British Columbia approved this study.

### Clinical Phenotype

The primary outcome variable evaluated in this study was 28-day mortality. Various organ dysfunctions were considered as secondary outcome variables. Baseline demographics recorded were age, gender, admission APACHE II score (KNAUS W A. et al. Crit Care Med (1985) 13:818-829), and medical or surgical diagnosis on admission to the ICU (based on the APACHE III diagnostic codes) (KNAUS W A. et al. Chest (1991) 100:1619-1636) (TABLE 2B).

TABLE 2B
Baseline characteristics key
Baseline Key

| | |
|---|---|
| AGE | Given In Years |
| GENDER | Percentage of Male Subjects |
| APACHE II | APACHE II score |
| % SURGICAL | The % of Subjects with a SURGICAL ICU admitting |
| | diagnosis |
| SEP.ADMIT | Sepsis upon admission |
| SEP.ANY | Sepsis anytime during admission |
| SS.ADMIT | Septic shock upon admission |
| SS.ANY | Septic shock anytime during admission |

After meeting the inclusion criteria, data were recorded for each 24-hour period (8 am to 8 am) for 28-days after ICU admission or until hospital discharge to evaluate organ dysfunction, the intensity of SIRS (Systemic Inflammatory Response Syndrome) and sepsis. Raw clinical and laboratory variables were recorded using the worst or most abnormal variable for each 24-hour period with the exception of Glasgow Coma Score, for which the best possible score for each 24-hour period was recorded. Missing data on the date of admission was assigned a normal value and missing data after day one was substituted by carrying forward the value from the previous day. When data collection for each patient was complete, all patient identifiers were removed from all records and the patient file was assigned a unique random number linked with the blood samples. The completed raw data file was used to calculate descriptive and severity of illness scores using standard definitions as described below.

Organ dysfunction was first evaluated at baseline and then daily using the Brussels score (SIBBALD W J. and VINCENT J L. Chest (1995) 107(2):522-7) (see TABLE 2A in General Methods Section). If the Brussels score was moderate, severe, or extreme dysfunction then organ dysfunction was recorded as present on that day. To correct for deaths during the observation period, we calculated the days alive and free of organ dysfunction (RUSSELL J A. et al. Crit Care Med (2000) 28(10):3405-11 and BERNARD G R. et al. Chest (1997) 112(1):164-72) (TABLE 2C). For example, the severity of cardiovascular dysfunction was assessed by measuring days alive and free of cardiovascular dysfunction over a 28-day observation period. Days alive and free of cardiovascular dysfunction was calculated as the number of days after inclusion that a patient was alive and free of cardiovascular dysfunction over 28-days. Thus, a lower score for days alive and free of cardiovascular dysfunction indicates more cardiovascular dysfunction. The reason that days alive and free of cardiovascular dysfunction is preferable to simply presence or absence of cardiovascular dysfunction is that severe sepsis has a high acute mortality so that early death (within 28-days) precludes calculation of the presence or absence of cardiovascular dysfunction in dead subjects. Organ dysfunction has been evaluated in this way in observational studies (RUSSELL J A. et al. Crit Care Med (2000) 28(10):3405-11) and in randomized controlled trials of new therapy in sepsis, acute respiratory distress syndrome (BERNARD G R. et al. N Engl J Med (1997) 336(13):912-8) and in critical care (HEBERT P C. et al. N Engl J Med (1999) 340(6):409-17).

To further evaluate cardiovascular, respiratory, and renal function we also recorded, during each 24-hour period, vasopressor support, mechanical ventilation, and renal support, respectively. Vasopressor use was defined as dopamine >5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis).

As a cumulative measure of the severity of SIRS, the presence of two, three or four of the SIRS criteria was scored each day over the 28-day observation period. SIRS was considered present when subjects met at least two of four SIRS criteria. The SIRS criteria were 1) fever (>38° C.) or hypothermia (<36° C.), 2) tachycardia (>90 beats/minute), 3) tachypnea (>20 breaths/minute), PaCO2 <32 mm Hg, or need for mechanical ventilation, and 4) leukocytosis (total leukocyte count >12,000/mm³) or leukopenia (<4,000/mm³).

TABLE 2C
Primary and Secondary Outcome Variables For ICU cohort and subsets
Survival and Days alive and free (DAF) of organ dysfunction Key

| | |
|---|---|
| SURVIVAL | 28-Day Survival |
| ALI.DAF | Days alive and free of acute Lung Injury |
| PRESS.DAF | Days alive and free of any vasopressors |
| PRESS2.DAF | Days alive and free of more than 2 g/μmin of |
| | vasopressors |
| PRESS5.DAF | Days alive and free of more than 5 μg/min of |
| | vasopressors |
| PRESS15.DAF | Days alive and free of more than 15 μg/min of |
| | vasopressors |
| INO.DAF | Days alive and free of inotropes |
| SIRS2.DAF | Days alive and free of 2 of 4 SIRS criteria |
| SIRS3.DAF | Days alive and free of 3 of 4 SIRS criteria |
| SIRS4.DAF | Days alive and free of 4 of 4 SIRS criteria |
| STER.DAF | Days alive and free of steroids |
| CVS.DAF | Days alive and free of cardiovascular dysfunction |
| RESP.DAF | Days alive and free of respiratory dysfunction |
| PF300.DAF | Days alive and free of PaO2/FiO2 less than 300 mmHg |
| VENT.DAF | Days alive and free of mechanical ventilators |
| CNS.DAF | Days alive and free of neurological dysfunction |
| COAG.DAF | Days alive and free of coagulation dysfunction |
| INR.DAF | Days alive and free of international normalized |
| | ratio >1.5 |
| ACRF.DAF | Days alive and free of acute renal failure |
| ANYREN.DAF | Days alive and free of any type of renal dysfunction |
| RENSUP.DAF | Days alive and free of renal support |
| ACHEP.DAF | Days alive and free of acute hepatic dysfunction |
| ANYHEP.DAF | Days alive and free of any type of hepatic dysfunction |
| AFFD.DAF | Days alive and free of acute failure |
| FFD.DAF | Days alive and free of acute or chronic failure |

### Selection of SNPs for Genotyping

Publicly available genotype data was queried from the International HapMap Project (www.hapmap.org) and Perlegen Sciences, Inc. (www.perlegen.com) to select a set of tag SNPs (tSNPs) in the SERPINE1 and PROC regions each having a minor allele frequency (MAF) greater than 0.05. These tSNPs were chosen using several statistical methods, including pairwise linkage disequilibrium (LD) measures (DEVLIN B. and RISCH N. Genomics (1995) 29:311-322), haplotype (STEPHENS M. et al. Am J Hum Genet. (2001) 68:978-989; and EXCOFFIER L. and SLATKIN M. Mol. Biol. Evol. (1995) 12(5):921-927) and haplotype block (HAWLEY M E. and KIDD K K. J. Heredity. (1995) 86:409-411) patterns, as well as phylogenetic (cladistic) distance metrics (HAWLEY M E. and KIDD K K. (1995)).

### Sample Analysis

### Sample Preparation

Discarded whole blood samples, stored at 4° C., were collected from the hospital laboratory. DNA was extracted from buffy coat using the QIAamp DNA Midi kit (Qiagen, Mississauga, ON, Canada). After extraction, the DNA samples were transferred to 1.5 mL cryotubes, bar coded and cross-referenced with a unique patient number and stored at −80° C.

### ABI Genotyping

Single nucleotide polymorphisms in SERPINE1 and PROC were genotyped using the 5′ nuclease, Taqman™ (Applied Biosystems; Foster City, Calif.) polymerase chain reaction (PCR) method.

### Illumina Genotyping

Single nucleotide polymorphisms in SERPINE1 and PROC were genotyped using the Illumina Golden Gate™ assay from 250 ng of DNA extracted from buffy coat. A list of these SNPs can be found labeled as cohort ‘I’ in TABLE 1B found in the General Methods section.

### Linkage Disequilibrium Analysis

Included in this patent are SNPs found to be associated with 28-day survival or response to XIGRIS™ as well as SNPs determined to be in LD with the former. LD SNPs were ascertained using either Haploview (BARRETT J C. et al. Bioinformatics (2005) 21(2):263-5 (http://www.broad.mit.edu/mpg/haploview/)) or the LD function in the Genetics Package in R (R Core Development Group, 2005-R Development Core Team (www.R-project.org). A R² threshold of 0.5 was required in order that a SNP be considered in LD with those claimed herein. All LD SNPs are shown in TABLE 1B.

b. Activated Protein C Worldwide Evaluation in Severe Sepsis (PROWESS) Cohort

This study was conducted as a double-blind, randomized, placebo-controlled multicentre trial (BERNARD G R et al. (2001) N Engl J Med 344(10):699-709). Study subjects with severe sepsis were enrolled and randomized to receive either placebo or XIGRIS™. Severe sepsis was defined as having a known or suspected infection at the time of screening, having at least three SIRS criteria and at least one new organ dysfunction. Baseline characteristics including demographic variables, preexisting conditions, organ dysfunction, disease severity and laboratory indices were evaluated within the 24 hours before the start of the infusion. Patient samples were anonymized using a one-way encryption process that effectively strips personal identifiers from the record. The study endpoint and primary outcome variable was defined prospectively as death from any cause and assessed for 28 days after the start of therapy. Days alive and free (DAF) of organ dysfunction were defined as secondary outcome variables and were scored using the Sepsis-related Organ Failure Assessment (SOFA) system. Two other organ dysfunctions were measured in this study in addition to those evaluated using SOFA criteria. These included DAF of vasopressors and DAF of mechanical ventilation. During each 24-hour period, DAF was scored for each organ dysfunction measure with a score of 1 being assigned if the patient was alive and free of organ dysfunction. A score of 0 was assigned if the patient developed organ dysfunction or died during that 24-hour period.

### Adverse Event Methodology

An adverse event was defined as any undesirable experience or unanticipated benefit including pregnancy that occurred after the patient received study drug regardless of its relationship to the study drug or treatment group assignment. During the pre-infusion period, study site personnel assessed each enrolled patient and noted the occurrence and nature of presenting and preexisting conditions. Throughout the study period, study site personnel reassessed the patient and noted any change in the presenting and preexisting conditions, and the occurrence and nature of any adverse events. Lack of drug effect is not an adverse event in clinical trials. The purpose of the clinical trial was to establish drug effect.

All adverse events were recorded from the initiation of study drug infusion and up to, but not after, 28 days (672 hours) following the initiation of study drug infusion. An adverse event occurring within the 28-day study period, which at a later time met the criteria of a serious adverse event, was required to be reported as a serious adverse event. If an adverse event worsened in severity after the 28-day study period and became known to the investigator, the investigator was required to report it.

Investigators determined relatedness of an event to study drug based on a temporal relationship to study drug infusion as well as if the event was unexpected or unexplained given the patient's clinical course, previous medical conditions, and concomitant medications. An event was recorded as “drug-related” if the investigator believed it was reasonably related to study drug.

Adverse events for this study were analyzed in a treatment-emergent manner. Treatment emergent adverse events, also called treatment-emergent signs and symptoms (TESS), are those events that occurred or worsened (if present at baseline) after the start of study drug administration. Since rhAPC may have antithrombotic and profibrinolytic properties, adverse events that were also considered bleeding events were assessed as a subset of all adverse events.

Treatment-emergent adverse events and serious adverse events were reported through Study Day 28. The treatment-emergent adverse events and serious adverse events that first occurred or were ongoing during the study drug infusion period were also assessed as a subset of all events occurring during the 28-day study period. The study drug infusion period for each patient was defined as the date of initiation of study drug administration to the date of last study drug discontinuation plus the next calendar day.

An event was classified as a treatment-emergent adverse event during the study drug infusion period if the following occurred: (1) the event was a new event with onset during the study drug infusion period and the event onset was on or before Study Day 6, or (2) the event was a preexisting condition (i.e., ongoing at the start of study drug infusion) that worsened in severity on or before Study Day 6.

An event was classified as a serious adverse event during the study drug infusion period if the following occurred: (1) the event was a new event with onset during the study drug infusion period, the event onset was on or before Study Day 6, and the event became serious at any time during the 28-day study period, or (2) the event was a preexisting condition (i.e., ongoing at the start of study drug infusion) that became serious at any time during the 28-day study period.

Actual adverse events recorded were bleeding events including anemia hemorrhage, cerebral hemorrhage, duodenal ulcer hemorrhage, esophageal hemorrhage, gastrointestinal hemorrhage, hemolysis, hemoperitoneum, hemoptysis, hemorrhagic colitis, hemothorax, lung hemorrhage, melena, muscle hemorrhage, rectal hemorrhage, rupture of spleen and thrombotic events including arterial thrombosis, cerebral arterial thrombosis, cerebral infarct, cerebrovascular accident, deep thrombophlebitis, embolus, myocardial infarct, pulmonary embolus, pulmonary thrombosis, and thrombosis.

### Protein Assay Methodology

For all study subjects, blood samples were drawn at pre-infusion (Day 0) and on study days 1-7, 14 and 28 for protein C (PC) measurements. PC levels were measured on a STA coagulation analyzer using the STA-Staclot Protein C kit (Diagnostica Stago, Asnieres-Sur-Seine, France). The statistical analysis of the association between PC levels and genotype was performed on a subset of subjects who had a PC measurement at baseline and genotype data available.

In the last 403 consecutively-enrolled patients, blood samples were drawn at pre-infusion (Day 0) and on study days 1, 2, 4 and 5 for PAI-1 measurements. PAI-1 levels were measured on citrated plasma samples using chromogenic activity assays on either STA or STA Compact coagulation analyzers (Diagnostica Stago Inc., Asnieres, France). The statistical analysis of the association between PAI-1 levels and genotype was undertaken on a subset of subjects with PAI-1 levels and available genotype data.

### Genotyping

DNA was extracted from blood spotted on Whatman FTA cards and genotyped for polymorphisms in PROC using the iPLEX platform (Sequenom Inc.). TABLE 2D contains the NCBI rs identifier numbers (rs ID), the chromosomal position of each SNP genotyped and the alleles observed. Patients included in the analysis for rs7242 are those that were successfully genotyped in both rs7242 and rs2069912. Patients included in the analysis for rs11178 are those that were successfully genotyped in both rs11178 and rs2069912. Patients included in the analysis for rs2227706 are those that were successfully genotyped in both rs2227706 and rs2069912. Patients included in the analysis for rs2227684 are those that were successfully genotyped in both rs2227684 and rs2069912. In a small number of patients, missing SNP data was imputed using the LD SNP using the method presented in “Missing Data Imputation, Classification, Prediction and Average Treatment Effect Estimation via Random Recursive Partitioning” (February 2006) IACUS, Stefano Maria and PORRO, Giuseppe (Available at SSRN: http://ssrn.com/abstract=905143).

TABLE 2D
SERPINE 1 and PROC SNPs genotyped in this study

| | chromosomal position (NCBI | |
|---|---|---|
| rsID | Build 36) | Observed Alleles |
| rs7242 | 100568165 | G/T |
| rs11178 | 100567804 | C/T |
| rs2227706 | 100570015 | A/G |
| rs2227684 | 100563651 | A/G |
| rs2069912 | 127894661 | T/C |

### 4. Statistical Analysis

a. Activated Protein C Worldwide Evaluation in Severe Sepsis (PROWESS) Cohort

### 28-day Survival and Response to XIGRIS™:

Logistic regression was performed using the STATS package in R (The R Project for Statistical Computing; http://www.r-project.org/) by genotype to test the following two null hypotheses using 28-day survival as the predictor variable:
a) genotype does not predict 28-day survival in the placebo treated patients
b) genotype does not predict response to administration of XIGRIS™ as measured by 28-day survival.

Individuals were stratified by treatment (i.e. placebo-treated or XIGRIS™-treated) and logistic regression analysis was undertaken first for all study participants (n=1490) and then by the following subgroups:
1. All subjects with APACHE II ≧25 (n=817)
2. All subjects with 2 or more major organ dysfunctions (MOD) (n=1271)

To test for differences in protein levels by genotype, repeated measures ANOVA were performed using SAS (SAS Institute, Cary, N.C.) to test the following four null hypotheses for protein C and PAI-1:
H₀: Mean protein C levels are the same for each genotype (rs7242 GG versus rs7242GT/rs7242 TT) within the placebo-treated PROWESS subjects.
H₀: Mean protein C levels are the same for each genotype (rs7242 GG versus rs7242GT/rs7242 TT) within the XIGRIS™-treated PROWESS subjects.
H₀: Mean PAI-1 levels are the same for each genotype (rs7242 GG versus rs7242GT/rs7242 TT) within the placebo-treated PROWESS subjects.
H₀: Mean PAI-1 levels are the same for each genotype (rs7242 GG versus rs7242GT/rs7242 TT) within the XIGRIS™-treated PROWESS subjects.

### Adverse Events in Placebo- or XIGRIS™-Treated Subjects

We assessed the incidence of adverse events in the PROWESS cohort in two ways. First, to ask whether subjects within a treatment group had a different incidence of adverse events by genotype, we employed a logistic regression approach with the STATS package in R (The R Project for Statistical Computing; http://www.r-project.org). We also modeled the adverse events data using an Fisher's exact test approach which allowed us to ask if subjects within a genotype group had a different incidence of adverse events given a particular treatment.

b. SPH Cohort

All data analysis was carried out using statistical packages available in R(R Core Development Group, 2005-R Development Core Team (www.R-project.org). R: A language and environment for statistical computing. Vienna, Austria. 2005). Either a Chi-square or Kruskal Wallis approach was used to identify significantly different baseline characteristics (age, gender, admitting APACHE II score, and medical vs. surgical admitting diagnosis) requiring post-hoc, multivariate adjustment. The Kruskal-Wallis test used in R (Hmisc package) computes a p value based on the F distribution. Median and quartiles (i.e. 25^{th} and 75^{th} percentiles) for days alive and free (DAF) of various measures of organ dysfunction were calculated and assessed for significance using a Kruskal Wallis approach. To evaluate whether genotype predicts differential response to treatment, differences in DAF of various organ dysfunction measures were compared by genotype within treatment group.

Logistic regression was performed using the STATS package in R by genotype to test the following two null hypotheses using 28-day survival as the predictor variable:
a) genotype does not predict 28-day survival in the control patients
b) genotype does not predict response to administration of XIGRIS™ as measured by 28-day survival.

A guide to the interpretation of the logistic regression analysis output is shown in TABLE 2E below.

TABLE 2E
Logistic regression interpretation key

| | |
|---|---|
| base | The genotype to which all other genotypes are compared. |
| log odds | The change in log odds of survival for the specified term. |
| | The direction of the log odds correspond to an increased |
| | risk of death (negative log odds) or decreased risk of death |
| | (positive log odds) for the specified term. |
| std error | The standard error associated with the estimate of log odds |
| SNP term | Estimate of the risk of death by genotype in the placebo |
| | treated (PROWESS cohort) or control patients (SPH cohort) |
| Treatment | Estimate of the risk of death by treatment in the baseline |
| term | genotype patients |
| SNP* | Estimate of the interaction between SNP and treatment as a |
| Treatment | measure of the difference in treatment response relative |
| | to the base genotype group |
| model | genotype model used for comparison |

We considered the SNP*Treatment effect to be significant if a p-value <0.20 for the difference in 28-day survival was seen in both the PROWESS and SPH cohorts. When this criteria was met, we considered the allele or genotype predicting increased 28-day survival with XIGRIS™ treatment to be an “Improved Response Genotype” (IRG) or an “Improved Response Combination Genotype” (IRCG).

### EXAMPLES

### Example 1

### rs7242 and rs2070682 Genotypes are Predictive of Risk of Death Response to XIGRIS™ and Risk of Organ Dysfunction in Cohorts of Subjects with Severe Sepsis

1.1.1: rs7242 is Predictive of Survival and Response to XIGRIS™ in the PROWESS Severe Sepsis Cohort All Subjects

TABLE 3 and 4 show baseline characteristics for all PROWESS placebo- and XIGRIS™-treated subjects genotyped for rs7242. With the exception of a difference in the distribution of APACHE II scores within the placebo-treated group, no significant differences by rs7242 genotype are observed.

TABLE 3
Baseline Characteristics for placebo-treated PROWESS subjects by
rs7242 genotype. 25^{th} percentile, median and 75^{th} percentile values
are given for each baseline characteristic.

| | rs7242 genotype (n) | | | ProbF P | |
|---|---|---|---|---|---|
| | GG (141) | GT (331) | TT (280) | Value | X² P value |
| Age (years) | 48.9/61.8/73.4 | 49.6/64.2/74.1 | 48.8/62.6/72.8 | 0.9343 | 0.7957 |
| Sex (% male) | 61.0 | 56.8 | 58.6 | NA | 0.6926 |
| Surgical (%) | 31.2 | 28.0 | 33.2 | NA | 0.3679 |
| APACHE II | 17.0/23.0/29.0 | 20.0/24.0/30.0 | 20.0/25.0/31.0 | 0.0430 | 0.0724 |

TABLE 4
Baseline Characteristics for XIGRIS ™-treated PROWESS subjects
by rs7242 genotype. 25^{th} percentile, median and 75^{th} percentile values are
given for each baseline characteristic.

| | rs7242 genotype (n) | | | ProbF P | |
|---|---|---|---|---|---|
| | GG (145) | GT (343) | TT (263) | Value | X² P value |
| Age (years) | 50.2/65.3/75.7 | 50.357/64.375/75.134 | 49.1/64.1/74.8 | 0.6265 | 0.6624 |
| Sex (% male) | 54.5 | 58.6 | 53.6 | NA | 0.4310 |
| Surgical (%) | 23.1 | 30.9 | 32.6 | NA | 0.1246 |
| APACHE II | 19.0/24.0/31.0 | 19.0/24.0/29.0 | 20.0/25.0/29.0 | 0.2564 | 0.3437 |

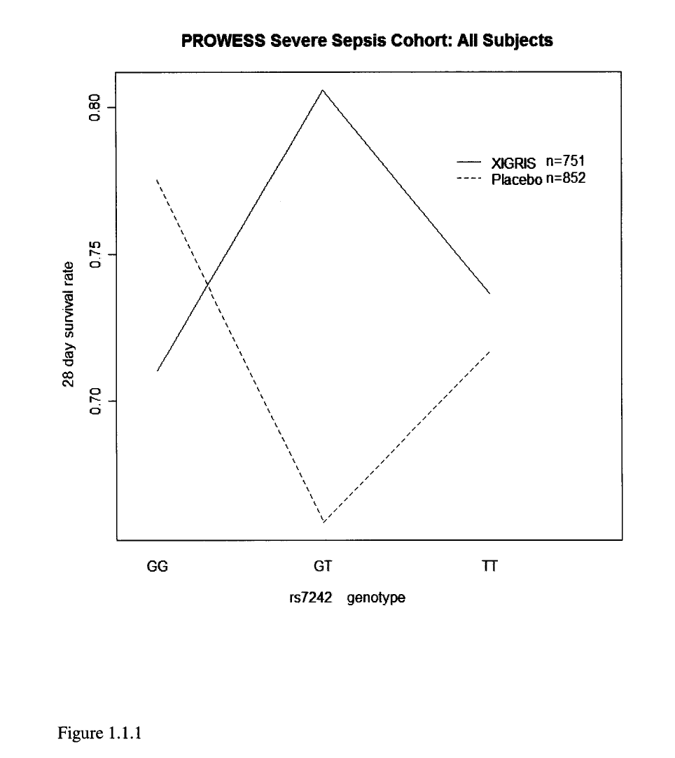

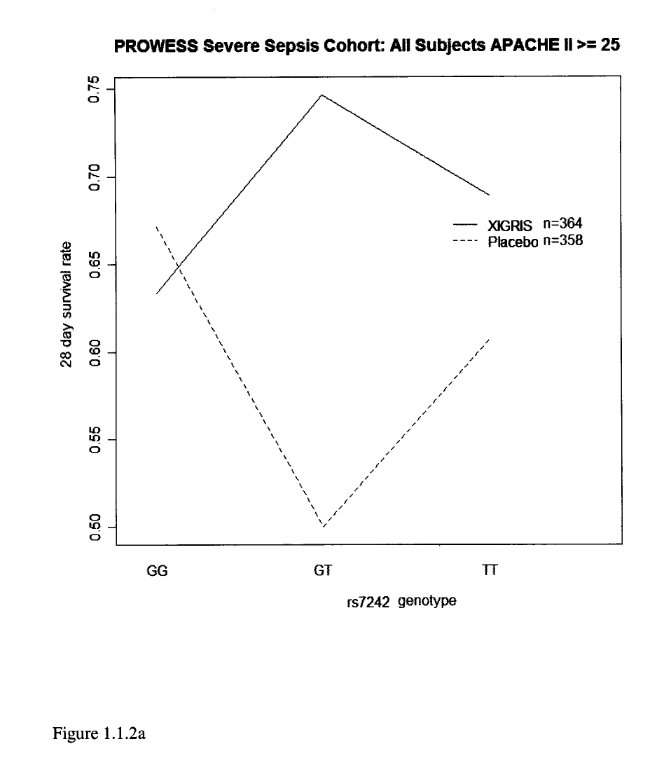

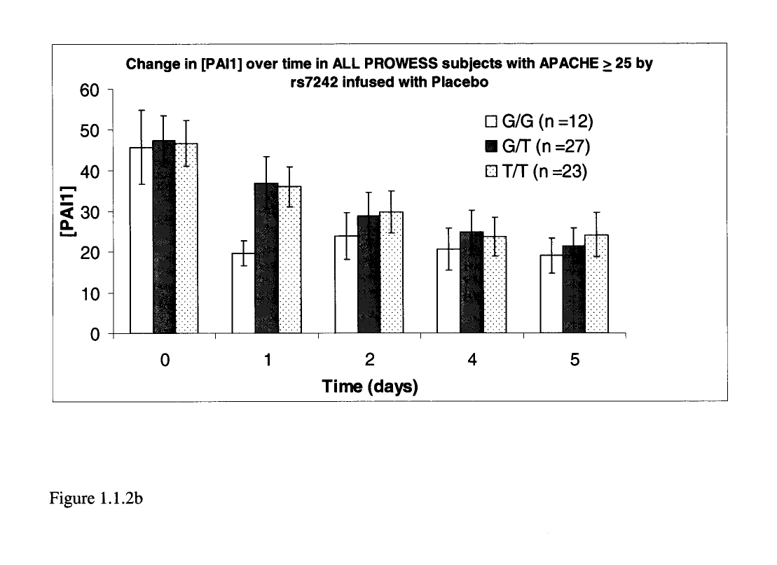

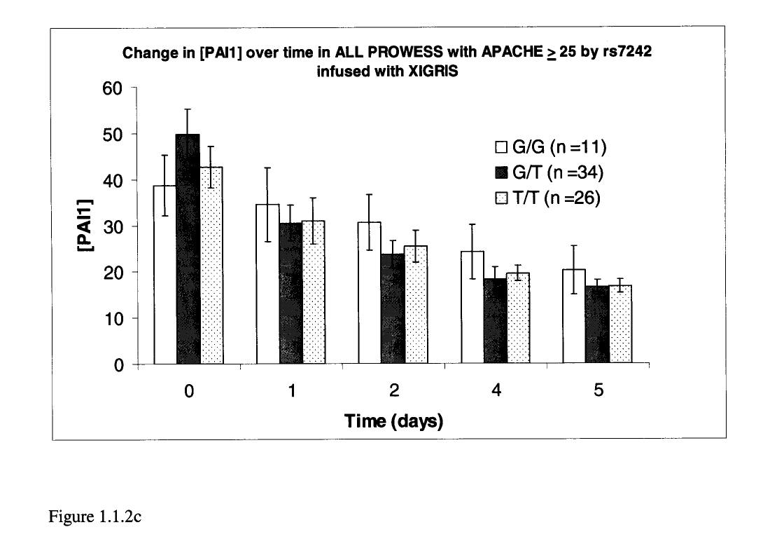

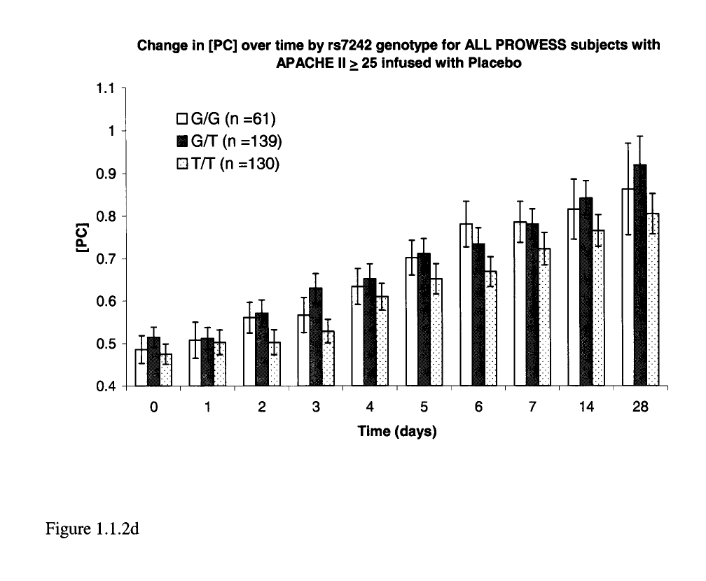

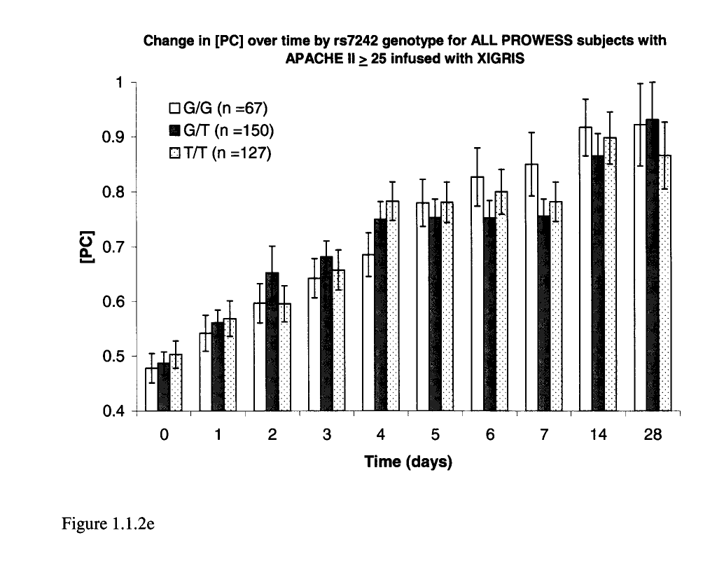

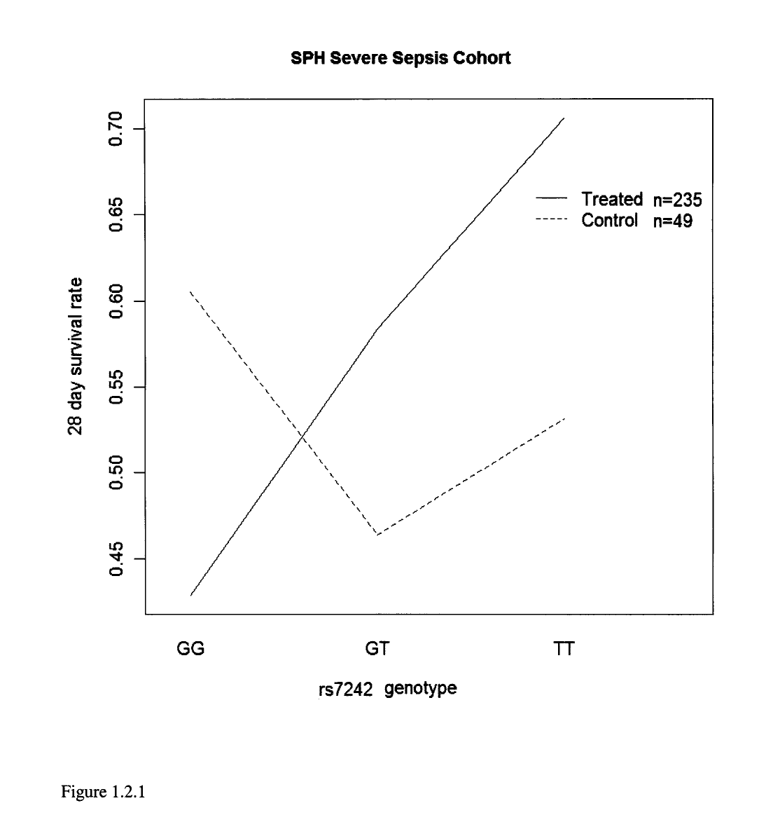

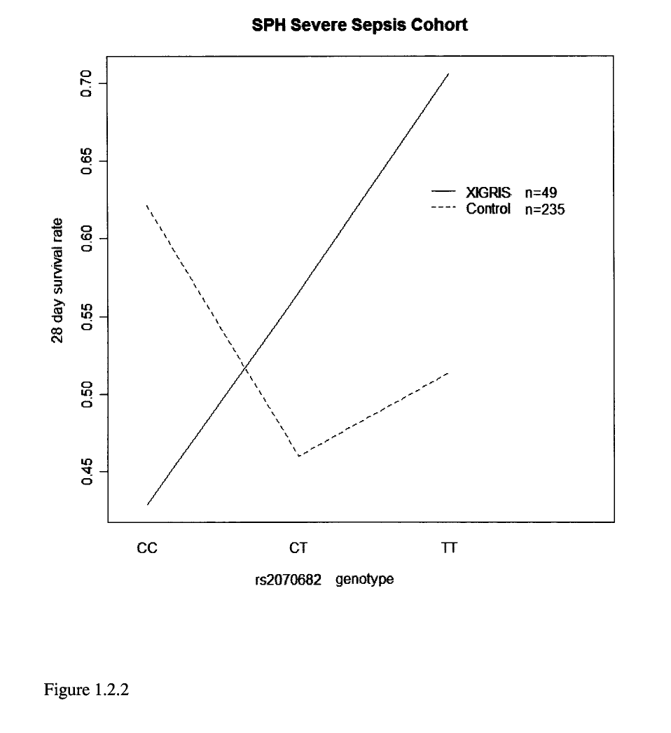

TABLE 5 shows percent survival by rs7242 genotype and treatment for all patients genotyped for rs7242 the PROWESS Severe Sepsis cohort. FIG. 1.1.1 illustrates the genotype distributions from this table in graphical form. TABLE 6 shows logistic regression statistics comparing risk of death and response to XIGRIS™ by rs7242 genotypes for all subjects using the genotype distributions detailed in TABLE 5. In the absence of XIGRIS™ treatment, rs7242 GG subjects have a decreased risk of death compared to those who are rs7242 GT or TT (p=0.0671). In contrast, when treated with XIGRIS™, rs7242 GT or TT subjects have improved survival compared with those who are GG (p=0.0136) with GT subjects having the most improved response (p=0.0012).

TABLE 5
28-day survival by rs7242 genotype and treatment for all PROWESS
subjects; data is presented as Nsurvived/Ntotal (% Survived)

| | rs7242 | | |
|---|---|---|---|
| | GG | GT | TT |
| Placebo | 108/141 (76%) | 219/331 (66%) | 201/280 (72%) |
| XIGRIS ™ | 103/145 (71%) | 277/343 (81%) | 193/263 (73%) |
| Percent Change | −5.0 | +15 | +1 |
| (XIGRIS ™- | | | |
| Placebo) | | | |

TABLE 6
rs7242 logistic regression statistics: rs7242 GG vs GT/TT for all
PROWESS subjects

| | Estimate | St. | | genotype |
|---|---|---|---|---|
| | of Log Odds | Error | P value | model (base) |
| GT/TT | −0.3976 | 0.2172 | 0.0671 | recessive (GG) |
| Treatment | −0.2886 | 0.2703 | 0.2858 | recessive (GG) |
| GT/TT * Treatment | 0.7407 | 0.2703 | 0.0136 | recessive (GG) |
| GT | −0.5821 | 0.2349 | 0.0132 | categorical (GG) |
| TT * Treatment | 0.3689 | 0.3319 | 0.2664 | categorical (GG) |
| GT * Treatment | 1.0524 | 0.3246 | 0.0012 | categorical (GG) |

1.1.2: rs7242 is Predictive of Survival and Response to XIGRIS™ in the PROWESS Severe Sepsis Cohort: All PROWESS Subjects with APACHE II ≧25

TABLE 7 and 8 show baseline characteristics for all APACHE II ≧25 PROWESS placebo- and XIGRIS™-treated subjects genotyped for rs7242. No significant differences by rs7242 genotype are observed.

TABLE 7
Baseline Characteristics for placebo-treated PROWESS subjects
with APACHE II ≧ 25 by rs7242 genotype. 25^{th} percentile, median and
75^{th} percentile values are given for each baseline characteristic.

| | rs7242 genotype (n) | | | ProbF P | |
|---|---|---|---|---|---|
| | GG (65) | GT (152) | TT (141) | Value | X² P value |
| Age (years) | 55.9/70.3/75.0 | 54.3/69.2/76.1 | 57.1/67.3/74.5 | 0.8385 | 0.9727 |
| Sex (% male) | 63.1 | 59.2 | 58.9 | NA | 0.8341 |
| Surgical (%) | 21.5 | 27.0 | 31.9 | NA | 0.2897 |
| APACHE II | 27.0/30.0/32.0 | 28.0/31.0/35.0 | 27.0/31.0/36.0 | 0.1804 | 0.1958 |

TABLE 8
Baseline Characteristics for XIGRIS ™-treated PROWESS subjects
with APACHE II ≧ 25 by rs7242 genotype. 25^{th} percentile, median
and 75^{th} percentile values are given for each baseline characteristic.

| | rs7242 genotype (n) | | | ProbF P | |
|---|---|---|---|---|---|
| | GG (71) | GT (160) | TT (133) | Value | X² P value |
| Age (years) | 54.6/67.8/75.9 | 55.5/68.5/76.5 | 53.2/67.8/74.9 | 0.7483 | 0.6365 |
| Sex (% male) | 53.5 | 61.3 | 56.4 | NA | 0.4925 |
| Surgical (%) | 22.9 | 25.8 | 28.2 | NA | 0.7047 |
| APACHE II | 28.0/31.0/34.0 | 27.0/29.0/33.0 | 27.0/29.0/33.0 | 0.3400 | 0.1495 |

TABLE 9 shows percent survival by rs7242 genotype and treatment for all subjects with APACHE II ≧25 in the PROWESS Severe Sepsis cohort. FIG. 1.1.2a illustrates the genotype distributions from TABLE 9 in graphical form. TABLE 10 shows logistic regression statistics comparing risk of death and response to XIGRIS™ by rs7242 genotypes for all subjects with APACHE II ≧25 using the genotype data from TABLE 9. In the absence of XIGRIS™ treatment, there is a trend towards decreased risk of death for rs7242 GG subjects compared to those who are rs7242 GT or TT (p=0.1223) In contrast, when treated with XIGRIS™, rs7242 GT and TT subjects are observed to have improved survival over those who are rs7242 GG (p=0.0357) with the GT subjects having the most improved response (p=0.0012).

TABLE 9
28-day survival by rs7242 genotype and treatment for all PROWESS
subjects with APACHE ≧ 25; data is presented as Nsurvived/
Ntotal (% Survived)

| | rs7242 | | |
|---|---|---|---|
| | GG | GT | TT |
| Placebo | 43/65 (66%) | 77/152 (51%) | 86/141 (61%) |
| XIGRIS ™ | 45/71 (63%) | 120/160 (75%) | 91/133 (68%) |
| Percent Change | −3 | +24 | +7 |
| (XIGRIS ™-Placebo) | | | |

TABLE 10
rs7242 logistic regression statistics: rs7242 GG vs GT/TT for all
PROWESS subjects APACHE II ≧ 25

| | Estimate | St. | | |
|---|---|---|---|---|
| | of Log Odds | Error | P value | model (base) |
| GT/TT | −0.4439 | 0.2873 | 0.1223 | recessive (GG) |
| Treatment | −0.1216 | 0.3597 | 0.7353 | recessive (GG) |
| GT/TT * Treatment | 0.8405 | 0.4002 | 0.0357 | recessive (GG) |
| GT | −0.5151 | 0.2303 | 0.0254 | categorical (GG) |
| GT * Treatment | 1.0524 | 0.3246 | 0.0012 | categorical (GG) |

FIG. 1.1.2b and FIG. 1.1.2c show the change in SERPINE1 (PAI-1) protein levels over time by rs7242 genotype for all PROWESS subjects with APACHE II ≧25 infused with Placebo or XIGRIS™ respectively. In the absence of XIGRIS™ treatment, rs7242 GG individuals are in general, observed to have lower PAI-1 levels than subjects who are rs7242 GT or TT. In contrast, in XIGRIS™ treated subjects, all PAI-1 levels are observed to decrease independent of genotype. However, consistent with our model, PAI-1 levels from rs7242 GT and TT individuals are generally observed to decrease more quickly than PAI-1 levels for rs7242 GG subjects.

FIG. 1.1.2d and 1.1.2e show the change in protein C (PC) levels over time by rs7242 genotype for all PROWESS subjects with APACHE II ≧25 infused with Placebo or XIGRIS™ respectively. Under a dominant model, rs7242 TT/GT placebo-treated subjects are observed to have significantly different PC levels than those who are rs7242 GG (p=0.001). Furthermore, the PC levels of rs7242 TT/GT subjects have a significantly different response over time (p=0.0043) with the greatest difference in PC levels observed at day 6 and days 14 through 28.

1.1.3: rs7242 is Predictive of Survival and Response to XIGRIS™ in the PROWESS Severe Sepsis Cohort: All PROWESS Subjects with Two or More Organ Dysfunctions

TABLE 11 shows percent survival by rs7242 genotype and treatment for PROWESS Severe Sepsis subjects with two or more organ dysfunctions. TABLE 12 shows logistic regression statistics comparing risk of death and response to XIGRIS™ by rs7242 genotypes for all subjects with two or more organ dysfunctions using the genotype distributions detailed in TABLE 11. In the absence of XIGRIS™ treatment, rs7242 GG subjects have a decreased risk of death compared to those who are rs7242 GT or TT (p=0.1249). In contrast, when treated with XIGRIS™, rs7242 GT or TT subjects have improved survival compared with those who are GG (p=0.0162) with GT subjects having the most improved response (p=0.0016).

TABLE 11
28-day survival by rs7242 genotype and treatment for PROWESS
subjects with two or more organ dysfunctions; data is presented as
N-survived/N-total (% Survived)

| | rs7242 | | |
|---|---|---|---|
| | GG | GT | TT |
| Placebo | 98/130 (75%) | 198/302 (66%) | 187/260 (72%) |
| XIGRIS ™ | 93/134 (69%) | 250/312 (80%) | 176/240 (73%) |
| Percent Change | −6.0 | +16 | +1 |
| (XIGRIS ™-Placebo) | | | |

TABLE 12
rs7242 logistic regression statistics: rs7242 GG vs GT/TT for
PROWESS subjects with two or more organ dysfunctions

| | Estimate of | St. | | genotype |
|---|---|---|---|---|
| | Log Odds | Error | P value | model (base) |
| GT/TT | −0.3421 | 0.2229 | 0.1249 | recessive (GG) |
| Treatment | −0.3002 | 0.2768 | 0.2781 | recessive (GG) |
| GT/TT * Treatment | 0.7413 | 0.3084 | 0.0162 | recessive (GG) |
| GT | −0.4754 | 0.2369 | 0.0448 | categorical (GG) |
| TT * Treatment | 0.3712 | 0.3420 | 0.2777 | categorical (GG) |
| GT * Treatment | 1.0507 | 0.3338 | 0.0016 | categorical (GG) |

1.2.1: rs7242 Predicts Survival and Response to XIGRIS™ in SPH Severe Sepsis Cohort:

TABLES 13 and 14 show baseline characteristics by rs7242 genotype for SPH XIGRIS™-treated and control subjects respectively. No significant differences by rs7242 genotype are observed.

TABLE 13
Baseline Characteristics for Xigris-treated SPH subjects by rs7242
genotype. 25^{th} percentile, median and 75^{th} percentile
values are given for each baseline characteristic.

| | GG (n = 7) | GT/TT (n = 41) | P |
|---|---|---|---|
| AGE | 31.00\|40.00\|61.00 | 40\|52\|67 53.34 +/− 2.77 | 0.426 |
| | 46.43 +/− 7.75 | | |
| APACHEII | 22.5\|32\|33.5 29.00 +/− | 23\|30\|35 29.20 +/− 1.31 | 0.950 |
| | 2.76 | | |
| SEX | 2 (28.57%) | 25 (60.98%) | 0.235 |
| SURGICAL | 4 (57.14%) | 10 (24.39%) | 0.189 |

TABLE 14
Baseline Characteristics for SPH Control Subjects by rs7242 genotype.
25^{th} percentile, median and 75^{th} percentile values are given
for each baseline characteristic.

| | GG (n = 38) | GT/TT (n = 189) | P |
|---|---|---|---|
| AGE | 54.00\|64.00\|70.00 | 52\|65\|74 62.4 +/− 1.10 | 0.476 |
| | 60.53 +/− 2.37 | | |
| APACHEII | 26\|29\|32.75 29.63 +/− 0.66 | 26\|29\|33 30.47 +/ | 0.268 |
| | | − 0.36 | |
| SEX | 20 (52.63%) | 133 (70.37%) | 0.052 |
| SURGICAL | 8 (21.05%) | 41 (21.69%) | 0.897 |

TABLE 15 shows percent survival by rs7242 genotype for the SPH Severe Sepsis cohort. FIG. 1.2.1 illustrates the genotype distributions from this table in graphical form. TABLE 16 shows logistic regression statistics comparing risk of death and response to XIGRIS™ by rs7242 genotypes for SPH severe sepsis subjects using the genotype distributions in TABLE 15. In the absence of XIGRIS™ treatment, rs7242 GT and TT subjects have a higher risk of death than GG subjects (p=0.21). There is a trend towards improved survival with XIGRIS™ treatment by rs7242 genotype in the SPH severe sepsis cohort with rs7242 GT and TT individuals being better responders to XIGRIS™ than the GG individuals (p=0.15).

TABLE 15
28-day survival by rs7242 genotype and treatment group in the SPH
severe sepsis cohort. Data is presented as N-survived/N-total
(percent survived)

rs7242

| | GG | GT | TT |
|---|---|---|---|
| Control | 23/38 (61) | 51/110 (46) | 42/79 (53) |
| XIGRIS ™-treated | 3/7 (43) | 14/24 (58) | 12/17 (71) |
| Percent Change | −18 | +12 | +18 |
| (XIGRIS ™- | | | |
| Placebo) | | | |

TABLE 16
Logistic regression statistics for SPH Severe Sepsis subjects by
rs7242 genotype

| | Estimate | | | |
|---|---|---|---|---|
| | of Log Odds | St. Error | P value | model (base) |
| GT/TT | −0.459 | 0.362 | 0.21 | recessive (GG) |
| Treatment | −0.715 | 0.833 | 0.39 | recessive (GG) |
| GT/TT * Treatment | 1.297 | 0.905 | 0.15 | recessive (GG) |

TABLES 17 and 18 show organ dysfunction data by rs7242 genotype for XIGRIS™-treated and control subjects respectively. In general, rs7242 GG individuals treated with XIGRIS™ have more organ dysfunction as demonstrated by fewer days alive and fewer days alive and free (DAF) of coagulation dysfunction, liver dysfunction, poor international normalization ratio and renal support. In contrast, in the absence of XIGRIS™ treatment, rs7242 GG individuals are observed to have improved organ dysfunction compared to TT/GT individuals as demonstrated by more DAF of various forms of organ dysfunction.

TABLE 17
Organ dysfunction statistics for XIGRIS ™-treated subjects by rs7242
genotype in SPH Severe Sepsis cohort. 25^{th} percentile, median and
75^{th} percentile values are given for each organ dysfunction.

| | | | Wilcoxon test |
|---|---|---|---|
| Organ Dysfunction | TT/GT (n = 41) | GG (n = 7) | p-value |
| DA | 10.00/28.00/28.00 | 2.50/4.00/28.00 | 0.124 |
| COAG.DAF | 7.00/23.00/28.00 | 0.50/3.00/27.50 | 0.2 |
| LIVER.DAF | 5.00/27.00/28.00 | 2.00/4.00/23.50 | 0.171 |
| INR.DAF | 1.00/25.00/28.00 | 0.50/4.00/20.00 | 0.172 |
| RENSUP.DAF | 2.00/14.00/28.00 | 0.50/1.00/21.00 | 0.139 |

TABLE 18
Organ dysfunction statistics for Control subjects by rs7242 genotype in
SPH Severe Sepsis cohort. 25^{th} percentile, median and 75^{th} percentile
values are given for each organ dysfunction.

| Organ | | | Wilcoxon test |
|---|---|---|---|
| Dysfunction | GG (n = 38) | TT/GT (n = 189) | p-value |
| DA | 5.00/26.00/28.00 | 11.75/28.00/28.00 | 0.121 |
| CVS.DAF | 0.00/12.00/23.00 | 4.25/15.50/23.75 | 0.193 |
| RENAL.DAF | 0.00/0.00/7.00 | 0.00/8.00/26.75 | 0.001 |
| COAG.DAF | 3.00/22.00/28.00 | 10.25/25.00/28.00 | 0.125 |
| LIVER.DAF | 5.00/26.00/28.00 | 11.75/28.00/28.00 | 0.278 |
| CNS.DAF | 0.00/10.00/24.00 | 5.25/19.50/27.00 | 0.048 |
| TISSHYPO.DAF | 4.00/24.00/28.00 | 11.00/27.00/28.00 | 0.173 |
| INR.DAF | 3.00/18.00/28.00 | 8.25/22.50/28.00 | 0.298 |
| PRESS.DAF | 2.00/18.00/25.00 | 6.75/20.50/26.00 | 0.089 |
| PRESS2.DAF | 2.00/18.00/25.00 | 6.75/20.50/26.00 | 0.092 |
| PRESS5.DAF | 2.00/19.00/26.00 | 9.50/22.50/26.00 | 0.124 |
| ALI.DAF | 2.00/9.00/25.00 | 6.25/18.50/27.75 | 0.113 |
| RENSUP.DAF | 2.00/11.00/28.00 | 3.00/23.00/28.00 | 0.304 |
| INO.DAF | 4.00/22.00/28.00 | 7.50/28.00/28.00 | 0.13 |
| ANYREN.DAF | 0.00/0.00/1.00 | 0.00/0.00/19.25 | 0.075 |
| DIC.DAF | 4.00/23.00/28.00 | 11.75/28.00/28.00 | 0.068 |

TABLE 19 shows the differences in median DAF of organ dysfunction within rs7242 genotype groups by treatment. Overall, rs7242 GG XIGRIS™-treated subjects have more organ dysfunction than GG control subjects as evidenced by fewer DAF of various forms of organ dysfunction. In contrast, rs7242 GT or TT XIGRIS™-treated subjects have decreased organ dysfunction compared to GT or TT control subjects as shown by more DAF of various forms of organ dysfunction.

TABLE 19
Differences mean days alive and free of organ dysfunction for rs7242 genotype
between Control and XIGRIS ™ treated subjects

| | TT/GT | | | GG | | |
|---|---|---|---|---|---|---|
| | Control | Xigris | | Control | Xigris | |
| Organ Dysfunction | (n = 189) | (n = 41) | Difference | (n = 38) | (n = 7) | Difference |
| DA | 26 | 28 | −2 | 28 | 4 | +24 |
| CVS.DAF | 12 | 15 | −3 | 15.5 | 0 | +15.5 |
| RESP.DAF | 6 | 9 | −3 | 6.5 | 1 | +5.5 |
| RENAL.DAF | 0 | 2 | −2 | 8 | 0 | +8 |
| COAG.DAF | 22 | 23 | −1 | 25 | 3 | +22 |
| LIVER.DAF | 26 | 27 | −1 | 28 | 4 | +24 |
| CNS.DAF | 10 | 18 | −8 | 19.5 | 1 | +18.5 |
| TISSHYPO.DAF | 24 | 27 | −3 | 27 | 4 | +23 |
| INR.DAF | 18 | 25 | −7 | 22.5 | 4 | +18.5 |
| PRESS.DAF | 18 | 23 | −5 | 20.5 | 0 | +20.5 |
| PRESS2.DAF | 18 | 23 | −5 | 20.5 | 0 | +20.5 |
| PRESS5.DAF | 19 | 24 | −5 | 22.5 | 0 | +22.5 |
| PRESS15.DAF | 23 | 26 | −3 | 25.5 | 0 | +25.5 |
| ALI.DAF | 9 | 9 | 0 | 18.5 | 3 | +15.5 |
| RENSUP.DAF | 11 | 14 | −3 | 23 | 1 | +22 |
| INO.DAF | 22 | 28 | −6 | 28 | 3 | +25 |
| ANYREN.DAF | 0 | 0 | 0 | 0 | 0 | 0 |
| ANYLIVER.DAF | 20 | 24 | −4 | 24 | 4 | +20 |
| VENT.DAF | 1 | 7 | −6 | 6 | 0 | +6 |

1.2.2: rs2070682 Predicts Survival and Response to XIGRIS™ in SPH Severe Sepsis Cohort:

TABLES 20 and 21 show baseline characteristics by rs2070682 genotype. With the exception of a difference in the sex distribution for control subjects, no significant differences between rs2070682 genotype are observed at baseline.

TABLE 20
Baseline Characteristics for SPH XIGRIS ™-treated Subjects by rs2070682
genotype. 25^{th} percentile, median and 75^{th} percentile values are given
for each baseline characteristic.

| | rs2070682 genotype (n) | | |
|---|---|---|---|
| | CC (n = 7) | TT/TC (n = 40) | Test statistic |
| Age | 31.0/40.0/61.0 | 41.5/56.5/67.0 | F = 1 d.f. = 1.45 P = 0.322 |
| Sex | 29% (2) | 65% (26) | Chi-square = 3.28 d.f. = 1 P = 0.07 |
| ApacheII | 23.00/32.00/36.00 | 23.00/31.50/36.25 | F = 0.02 d.f. = 1.45 P = 0.895 |
| Surgical | 57% (4) | 25% (10) | Chi-square = 2.94 d.f. = 1 P = 0.0863 |
| Ss.Admit | 86% (6) | 90% (36) | Chi-square = 0.12 d.f. = 1 P = 0.734 |
| Ss.Any | 86% (6) | 92% (37) | Chi-square = 0.35 d.f. = 1 P = 0.553 |

TABLE 21
Baseline characteristics for SPH control-treated subjects by rs2070682 genotype.
25^{th} percentile, median and 75^{th} percentile values are given for
each baseline characteristic.

| | rs2070682 genotype (n) | | |
|---|---|---|---|
| | CC (n = 37) | GT/TT (n = 172) | Test statistic |
| Age | 52.00/62.00/70.00 | 50.75/63.00/73.00 | F = 0.56 d.f. = 1,207 P = 0.454 |
| Sex | 51% (19) | 69% (118) | Chi-square = 4.01 d.f. = 1 P = 0.0451 |
| APACHE II | 26/29/33 | 27/30/34 | F = 1.15 d.f. = 1,207 P = 0.285 |
| Surgical | 24% (9) | 22% (37) | Chi-square = 0.14 d.f. = 1 P = 0.708 |
| Ss.Admit | 73% (27) | 84% (145) | Chi-square = 2.68 d.f. = 1 P = 0.101 |
| Ss.Any | 84% (31) | 88% (152) | Chi-square = 0.59 d.f. = 1 P = 0.443 |

TABLE 22 shows percentage survival by rs2070682 genotype for the SPH Severe Sepsis cohort. FIG. 1.2.2 illustrates the genotype distributions from this table in graphical form. TABLE 23 shows logistic regression statistics comparing risk of death and response to XIGRIS™ by rs2070682 genotypes for SPH severe sepsis subjects using the genotype data from TABLE 22. In the absence of treatment with XIGRIS™, rs2070682 CC individuals are predicted to have increased survival compared to CT and TT individuals (p=0.128). In addition, a trend towards an improved response to XIGRIS™ is observed for the rs2070682 CT and TT subjects compared to CC subjects (p=0.134).

TABLE 22
28-day survival by rs2070682 genotype and treatment group in
the SPH severe sepsis cohort. Data is presented as
N-survived/N-total (percent survived)

| | rs2070682 | | |
|---|---|---|---|
| | CC | CT | TT |
| Control | 23/37 (62) | 46/100 (46) | 37/72 (51) |
| XIGRIS ™-treated | 3/7 (43) | 13/23 (56) | 12/17 (71) |
| Percent Change (XIGRIS ™- | −19 | +10 | +20 |
| Placebo) | | | |

TABLE 23
Logistic regression statistics for rs2070682 genotype
for XIGRIS ™-treated and Control subjects from
SPH Severe Sepsis cohort

| | Estimate of | | | |
|---|---|---|---|---|
| | Log Odds | St. Error | P value | model (base) |
| CT/TT | −0.5662 | 0.3717 | 0.128 | recessive (CC) |
| Treatment | −0.7841 | 0.8356 | 0.348 | recessive (CC) |
| CT/TT* Treatment | 1.3647 | 0.9100 | 0.134 | recessive (CC) |

TABLES 24 and 25 show organ dysfunction by rs2070682 genotype for XIGRIS™-treated and control subjects respectively. In general, when treated with XIGRIS™, rs2070682 CC individuals are observed to have more organ dysfunction than CT/TT individuals as demonstrated by fewer days alive and free of acute lung injury, coagulation dysfunction, renal failure and acute hepatic failure. In contrast, in the absence of XIGRIS™ treatment, CC individuals have less organ dysfunction than CT/TT individuals as demonstrated by more days alive and free of various organ dysfunction measures.

TABLE 24
Organ dysfunction statistics for XIGRIS ™-treated subjects by
rs2070682 genotype in SPH Severe Sepsis cohort. 25^{th} percentile,
median and 75^{th} percentile values are given for each organ dysfunction.

| Organ | | | |
|---|---|---|---|
| Dysfunction | CC (n = 7) | CT/TT (n = 40) | Test statistic and p value |
| Days Alive | 2.5/4.0/28.0 | 10.0/28.0/28.0 | Chi-square = 0.95 d.f. = 1 |
| | | | P = 0.329 |
| ALI.DAF | 1.50/3.00/11.00 | 2.00/13.00/24.25 | F = 1.73 d.f. = 1.45 P = 0.195 |
| COAG.DAF | 0.50/3.00/27.50 | 6.75/20.00/28.00 | F = 1.76 d.f. = 1.45 P = 0.191 |
| INR.DAF | 2.00/4.00/27.50 | 5.75/27.00/28.00 | F = 2.03 d.f. = 1.45 P = 0.161 |
| ACRF.DAF | 0.00/1.00/15.00 | 5.00/17.00/28.00 | F = 3.75 d.f. = 1.45 P = 0.0592 |
| ANYREN.DAF | 0.00/1.00/15.00 | 4.75/14.00/28.00 | F = 3 d.f. = 1.45 P = 0.09 |
| ACHEP.DAF | 2.0/4.0/23.5 | 5.0/27.5/28.0 | \|F = 1.92 d.f. = 1.45 P = 0.172 |

TABLE 25
Organ dysfunction statistics for control subjects by rs2070682 genotype
in SPH Severe Sepsis cohort. 25^{th} percentile, median and
75^{th} percentile values are given for each organ dysfunction.

| Organ | | | |
|---|---|---|---|
| Dysfunction | CC (n = 37) | CT/TT (n = 172) | Test statistic and p value |
| Days Alive | 13/28/28 | 5/25/28 | F = 2.47 d.f. = 1,207 P = 0.118 |
| ALI.DAF | 6.00/16.00/28.00 | 2.00/9.00/25.25 | F = 3.11 d.f. = 1,207 P = 0.079 |
| INO.DAF | 9/28/28 | 4/23/28 | F = 2.09 d.f. = 1,207 P = 0.15 |
| PF300.DAF | 0/1/8 | 0/0/5 | F = 1.78 d.f. = 1,207 P = 0.184 |
| CNS.DAF | 7/24/27 | 3/15/26 | F = 1.85 d.f. = 1,207 P = 0.175 |
| INR.DAF | 9.0/26.0/28.0 | 3.0/20.5/28.0 | F = 2.29 d.f. = 1,207 P = 0.132 |
| ACRF.DAF | 9.0/26.0/28.0 | 2.0/11.5/27.0 | F = 7.58 d.f. = 1,207 P = 0.00643 |
| ANYREN.DAF | 2/16/28 | 1/8/26 | F = 2.74 d.f. = 1,207 P = 0.0993 |

TABLE 26 shows the differences in median DAF of organ dysfunction within rs2070682 genotype groups by treatment. Overall, rs2070682 CC XIGRIS™-treated subjects have more organ dysfunction than CC control subjects as evidenced by fewer DAF of various forms of organ dysfunction. In contrast, rs2070682 CT or TT XIGRIS™-treated subjects have decreased organ dysfunction compared to CT or TT control subjects as shown by more DAF of various forms of organ dysfunction.

TABLE 26
Differences for median days alive and free of organ dysfunction for rs2070682
genotype between Control and XIGRIS ™ treated subjects

| | CC | | | CT/TT | | |
|---|---|---|---|---|---|---|
| | Control | XIGRIS ™ | Difference | Control | XIGRIS ™ | Difference |
| ALI.DAF | 16.00 | 3.00 | −13 | 9.00 | 13.00 | +4 |
| INR.DAF | 26.0 | 4.00 | −22 | 20.5 | 27.00 | +6.5 |
| ACRF.DAF | 26.0 | 1.00 | −25 | 11.5 | 17.00 | +5.5 |
| ANYREN.DAF | 16 | 1.00 | −15 | 8 | 14.00 | +6 |

### Example 2

### Risk of Death and Response to XIGRIS™ by rs11178, rs2227706 and 2227684 Genotypes in the Prowess Severe Sepsis Cohort, which are Observed to be in LD with rs7242

2.1.1 Risk of Death and Response to XIGRIS™ by rs2227684 Genotype in the PROWESS Severe Sepsis Cohort which is in LD with rs7242.

TABLES 27 and 28 show baseline characteristics by rs2227684 genotype for PROWESS placebo- and XIGRIS™-treated subjects respectively. With the exception of a difference in APACHE II scores for placebo-treated subjects, no significant differences by rs2227684 genotype are observed.

TABLE 27
Baseline Characteristics for placebo-treated PROWESS subjects by rs2227684
genotype. 25^{th} percentile, median and 75^{th} percentile values are given for
each baseline characteristic.

| | rs2227684 genotype (n) | | | ProbF P | |
|---|---|---|---|---|---|
| | AA (135) | AG (353) | GG (283) | Value | X² P value |
| Age (years) | 48.9/62.2/73.4 | 49.2/64.0/74.2 | 48.8/62.6/72.9 | 0.9619 | 0.8151 |
| Sex (% male) | 59.3 | 56.9 | 59.0 | NA | 0.8327 |
| Surgical (%) | 31.9 | 26.8 | 35.0 | NA | 0.0806 |
| APACHE II | 17.0/23.0/29.0 | 20.0/24.0/30.0 | 20.0/25.0/31.0 | 0.0420 | 0.0647 |

TABLE 28
Baseline Characteristics for XIGRIS ™-treated PROWESS subjects
by rs2227684 genotype. 25^{th} percentile, median and 75^{th} percentile
values are given for each baseline characteristic.

| | rs2227684 genotype (n) | | | ProbF P | |
|---|---|---|---|---|---|
| | AA (146) | AG (369) | GG (262) | Value | X² P value |
| Age (years) | 47.8/65.2/75.6 | 49.6/64.1/75.1 | 47.5/63.8/75.0 | 0.747 | 0.799 |
| Sex (% male) | 53.4 | 57.5 | 55.0 | NA | 0.665 |
| Surgical (%) | 22.9 | 29.8 | 32.4 | NA | 0.1318 |
| APACHE II | 19.0/24.0/31.0 | 18.0/24.0/29.0 | 20.0/24.5/29.0 | 0.4011 | 0.5192 |

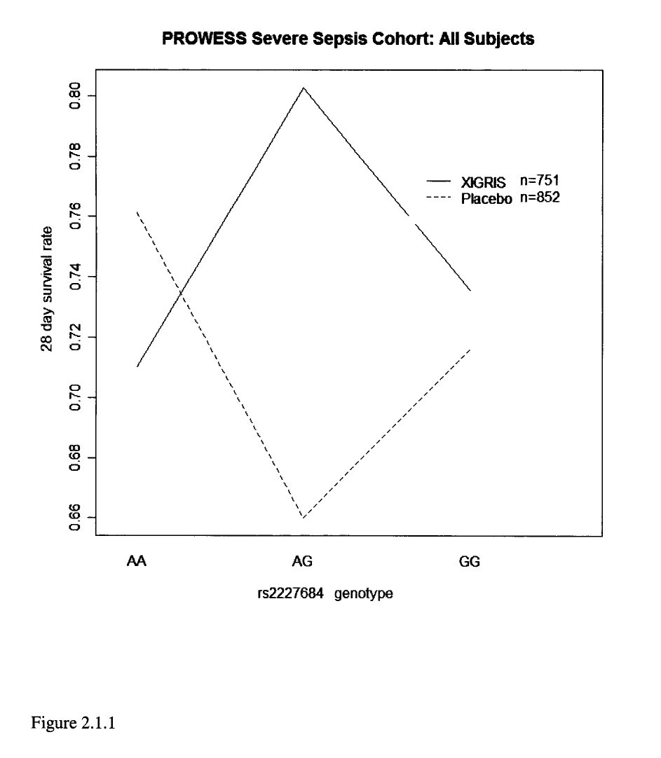

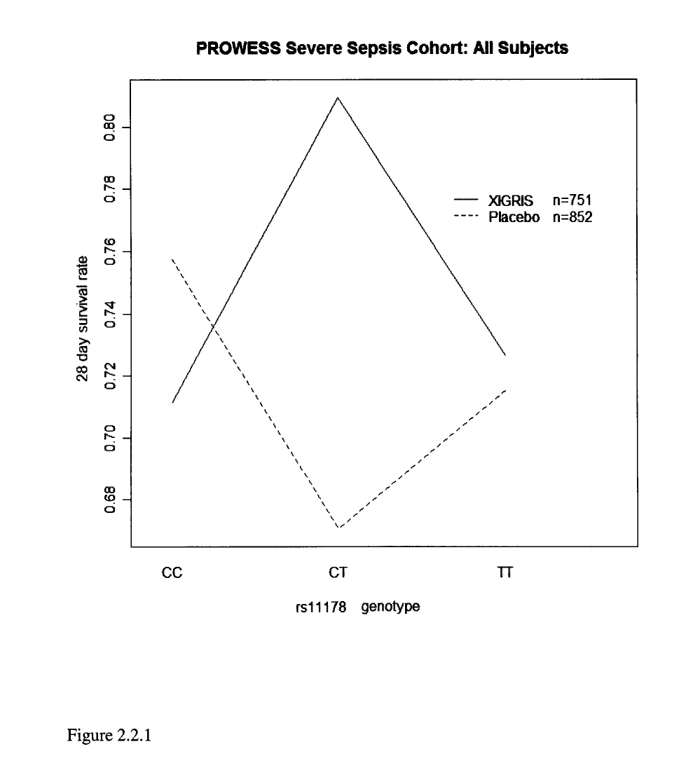

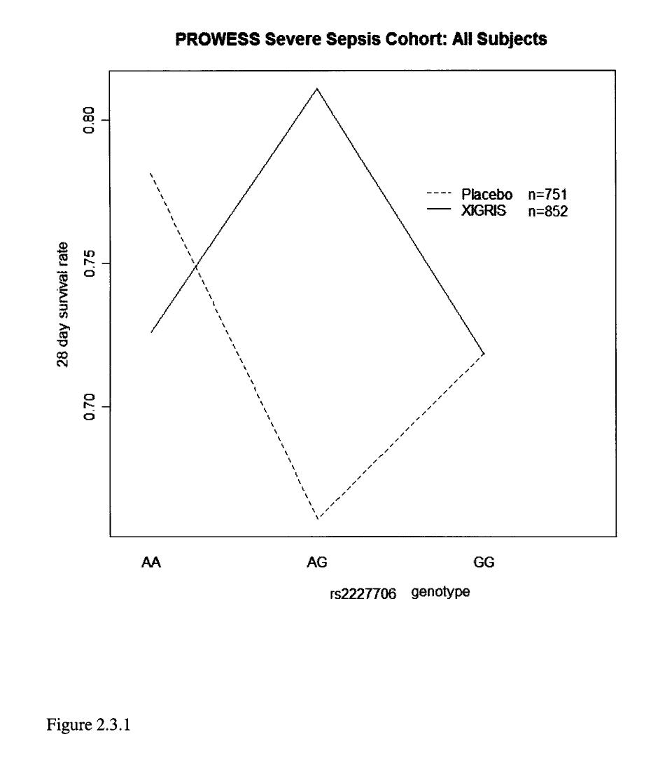

TABLE 29 shows percentage survival by rs2227684 genotype for all subjects in the PROWESS Severe Sepsis cohort. FIG. 2.1.1 illustrates the genotype distributions from this table in graphical form. TABLE 30 shows logistic regression statistics for rs2227684 AA subjects compared with AG and GG subjects using the data from TABLE 29. In the absence of XIGRIS™ treatment, rs2227684 AA subjects are observed to have a strong trend towards decreased risk of death compared to AG and GG subjects (p=0.1088). In contrast, when treated with XIGRIS™, rs2227684 GG and AG subjects are observed to have increased survival compared with AA subjects (p=0.0254) with AG subjects showing the strongest response (p=0.0029).

TABLE 29
28-day survival by rs2227684 genotype and treatment for all
subjects in the PROWESS severe sepsis cohort. Data is
presented as Nsurvived/Ntotal (% Survived)

| | rs2227684 | | |
|---|---|---|---|
| | AA | AG | GG |
| Placebo | 102/135 (76%) | 233/353 (66%) | 203/283 (72%) |
| XIGRIS ™ | 104/146 (71%) | 296/369 (80%) | 192/262 (73%) |
| Percent Change | −5 | _+14 | +1 |
| (XIGRIS ™-Placebo) | | | |

TABLE 30
Logistic regression statistics for all subjects in the PROWESS
severe sepsis cohort: rs2227684 AA vs AG/GG genotypes

| | Estimate of | St. | | |
|---|---|---|---|---|
| | Log Odds | Error | P value | model (base) |
| AG/GG | −0.3491 | 0.2177 | 0.1088 | recessive (AA) |
| Treatment | −0.2217 | 0.2712 | 0.4135 | recessive (AA) |
| AG/GG* Treatment | 0.6699 | 0.2998 | 0.0254 | recessive (AA) |
| AG | −0.4649 | 0.2296 | 0.0429 | categorical (AA) |
| AG* Treatment | 0.9581 | 0.3213 | 0.0029 | categorical (AA) |

2.1.2 Risk of Death and Response to XIGRIS™ by rs11178 Genotype in the PROWESS Severe Sepsis Cohort which is in LD with rs7242.

TABLE 31 and 32 show baseline characteristics by rs11178 genotype for PROWESS placebo- and XIGRIS™-treated subjects respectively. With the exception of a difference in APACHE II score in placebo-treated subjects, no significant differences by rs11178 genotype are observed.

TABLE 31
Baseline Characteristics for placebo-treated PROWESS subjects by rs11178
genotype. 25^{th} percentile, median and 75^{th} percentile values are given
for each baseline characteristic.

| | rs11178 genotype (n) | | | ProbF P | |
|---|---|---|---|---|---|
| | CC (138) | CT (348) | TT (282) | Value | X² P value |
| Age (years) | 48.3/61.63/73.43 | 49.4/64.94/74.64 | 48.84/62.6/72.7 | 0.7474 | 0.5602 |
| Sex (% male) | 60.1 | 57.5 | 58.9 | NA | 0.8520 |
| Surgical (%) | 32.6 | 27.7 | 34.4 | NA | 0.1839 |
| APACHE II | 17.0/23.0/29.0 | 20.0/24.0/30.0 | 20.0/25.0/31.0 | 0.0255 | 0.0388 |

TABLE 32
Baseline Characteristics for XIGRIS ™-treated PROWESS subjects
by rs11178 genotype. 25^{th} percentile, median and 75^{th} percentile values
are given for each baseline characteristic.

| | rs11178 genotype (n) | | | ProbF P | |
|---|---|---|---|---|---|
| | CC (150) | CT (360) | TT (268) | Value | X² P value |
| Age (years) | 48.6/65.2/75.7 | 50.0/64.4/75.1 | 47.5/64.0/74.9 | 0.6186 | 0.6710 |
| Sex (% male) | 52.7 | 58.1 | 54.1 | NA | 0.4398 |
| Surgical (%) | 24.3 | 29.6 | 32.5 | NA | 0.2235 |
| APACHE II | 19.0/24.0/31.0 | 19.0/24.0/29.0 | 19.5/24.5/29.0 | 0.2502 | 0.4164 |

Table 33 shows percentage survival by rs11178 genotype for all subjects in the PROWESS Severe Sepsis cohort. FIG. 2.2.1 illustrates the genotype distributions from this table in graphical form. TABLE 34 shows logistic regression statistics for rs11178 CC subjects compared with CT and TT subjects using the genotype data from TABLE 33. In the absence of XIGRIS™ treatment, a trend towards decreased risk of death is observed for rs11178 CC subjects compared with those who are CT or TT. In contrast, when treated with XIGRIS™, rs11178 CT and TT subjects are observed to have increased survival compared to CC subjects (p=0.0244) with CT subjects showing the strongest response (p=0.00197).

TABLE 33
28-day survival by rs11178 genotype and treatment for all subjects
in the PROWESS severe sepsis cohort). Data is presented as
Nsurvived/Ntotal (% Survived)

| | rs11178 | | |
|---|---|---|---|
| | CC | CT | TT |
| Placebo | 104/138 (75%) | 233/348 (67%) | 202/282 (71%) |
| XIGRIS ™ | 106/150 (71%) | 292/360 (81%) | 194/268 (72%) |
| Percent Change | −4 | +14 | +1 |
| (XIGRIS ™-Placebo) | | | |

TABLE 34
Logistic regression statistics for all subjects in the PROWESS
severe sepsis cohort: rs11178 CC vs CT/TT genotypes

| | Estimate of | St. | | |
|---|---|---|---|---|
| | Log Odds | Error | P value | model (base) |
| CT/TT | −0.3157 | 0.2155 | 0.1430 | recessive (CC) |
| Treatment | −0.2388 | 0.2668 | 0.3708 | recessive (CC) |
| CT/TT* Treatment | 0.6668 | 0.2962 | 0.0244 | recessive (CC) |
| CT | −0.4277 | 0.2304 | 0.063 | categorical (CC) |
| CT* Treatment | 0.9899 | 0.3199 | 0.00197 | categorical (CC) |

2.1.3 Risk of Death and Response to XIGRIS™ by rs2227706 Genotype in the PROWESS Severe Sepsis Cohort which is in LD with rs7242.

TABLE 35 and 36 show baseline characteristics by rs2227706 genotype for PROWESS placebo- and XIGRIS™-treated subjects respectively. With the exception of a difference in APACHE II score for placebo-treated subjects, no significant differences by rs2227706 genotype are observed.

TABLE 35
Baseline Characteristics for placebo-treated PROWESS subjects by
rs2227706 genotype. 25^{th} percentile, median and 75^{th} percentile values
are given for each baseline characteristic.

| | rs2227706 genotype (n) | | | ProbF P | |
|---|---|---|---|---|---|
| | AA (128) | AG (345) | GG (296) | Value | X² P value |
| Age (years) | 49.0/62.0/73.3 | 50.5/64.4/74.1 | 47.7/62.0/72.8 | 0.7803 | 0.5683 |
| Sex (% male) | 59.4 | 57.1 | 58.1 | NA | 0.901 |
| Surgical (%) | 32.8 | 27.1 | 34.5 | NA | 0.1186 |
| APACHE II | 17.0/23.0/29.0 | 20.0/24.0/30.0 | 20.0/25.0/31.0 | 0.0031 | 0.0066 |

TABLE 36
Baseline Characteristics for placebo-treated PROWESS subjects by rs2227706
genotype. 25^{th} percentile, median and 75^{th} percentile values are given
for each baseline characteristic.

| | rs2227706 genotype (n) | | | ProbF P | |
|---|---|---|---|---|---|
| | AA (136) | AG (352) | GG (293) | Value | X² P value |
| Age (years) | 49.1/66.3/75.3 | 50.8/65.7/75.5 | 45.8/62.4/73.6 | 0.1021 | 0.1304 |
| Sex (% male) | 50.0 | 57.7 | 55.6 | NA | 0.3118 |
| Surgical (%) | 23.1 | 29.1 | 32.4 | NA | 0.1505 |
| APACHE II | 20.0/24.0/31.0 | 18.5/24.0/29.0 | 19.0/24.029.0 | 0.3395 | 0.4899 |

TABLE 37 shows percentage survival by rs2227706 genotype for all subjects in the PROWESS Severe Sepsis cohort. FIG. 2.3.1 illustrates the genotype distributions from this table in graphical form. TABLE 38 shows logistic regression statistics for rs2227706 AA subjects compared with AG and GG subjects using genotype data from TABLE 37. In the absence of XIGRIS™-treatment, rs2227706 AA subjects are observed to have increased survival compared with AG and GG subjects (p=0.0362). In contrast, when treated with XIGRIS™, rs2227706 AG and GG subjects show improved survival compared with AA subjects (p=0.0277) with those who are AG showing the strongest response (p=0.0016).

TABLE 37
28-day survival by rs2227706 genotype and treatment for
all subjects in the PROWESS severe sepsis cohort. Data is
presented as N-survived/N-total (% Survived)

| | rs2227706 | | |
|---|---|---|---|
| | AA | AG | GG |
| Placebo | 100/128 (78%) | 228/345 (66%) | 213/296 (72%) |
| XIGRIS ™ | 99/136 (73%) | 285/352 (81%) | 210/293 (72%) |
| Percent Change | −5 | +15 | 0 |
| (XIGRIS ™-Placebo) | | | |

TABLE 38
Logistic regression statistics for all subjects in the PROWESS
severe sepsis cohort rs2227706 AA vs AG/GG genotypes

| | Estimate of | St. | | |
|---|---|---|---|---|
| | Log Odds | Error | P value | model (base) |
| AG/GG | −0.4822 | 0.2302 | 0.0362 | recessive (AA) |
| Treatment | −0.2888 | 0.2878 | 0.3157 | recessive (AA) |
| AG/GG* Treatment | 0.6920 | 0.3143 | 0.0277 | recessive (AA) |
| AG | −0.6058 | 0.2426 | 0.0124 | categorical (AA) |
| AG* Treatment | 1.0694 | 0.3412 | 0.0016 | categorical (AA) |

### Example 3

### Risk of Death and Response to XIGRIS™ by SERPINE1 rs7242 and PROC rs2069912 Genotype Combination

3.1.1 Risk of Death and Response to XIGRIS™ by SERPINE1 rs7242 and PROC rs2069912 Genotype Combination for All Subjects with APACHE II ≧25 in the PROWESS Severe Sepsis Cohort

TABLE 39 and 40 show baseline characteristics by SERPINE1 rs7242 and PROC rs2069912 genotype combination for PROWESS placebo- and XIGRIS™-treated subjects respectively. With the exception of a difference in APACHE II score for placebo-treated subjects, no significant differences by SERPINE1 rs7242 and PROC rs2069912 genotype combination are observed. TABLE 41 and 42 show baseline characteristics by SERPINE1 rs7242 and PROC rs2069912 genotype combination for PROWESS placebo- and XIGRIS™-treated subjects with APACHE ≧25 respectively. No significant differences by SERPINE1 rs7242 and PROC rs2069912 genotype combination are observed for the APACHE ≧25 subset.

TABLE 39
Baseline Characteristics for all placebo-treated PROWESS subjects
by PROC 2069912 and SERPINE 1 rs7242 combined genotype.
25^{th} percentile, median and 75^{th} percentile values are
given for age and APACHE II

| | rs2069912/rs7242 genotype combination (n) | | | |
|---|---|---|---|---|
| | IRGC (276) | MRGC (392) | NRGC (77) | P value |
| Age (years) | 51.8/64.9/73.6 | 47.1/61.5/73.3 | 50.9/64.1/73.4 | 0.3084 |
| Sex | 60.1 | 55.6 | 64.9 | 0.2308 |
| (% male) | | | | |
| Surgical (%) | 28.3 | 32.3 | 27.3 | 0.4448 |
| APACHE II | 21.0/24.5/31.0 | 19.0/24.0/30.0 | 17.0/23.0/30.0 | 0.0275 |

TABLE 40
Baseline Characteristics for all XIGRIS ™-treated PROWESS
subjects by PROC 2069912 and SERPINE 1 rs7242 combined
genotype. 25^{th} percentile, median and 75^{th} percentile
values are given for age and APACHE II

| | rs2069912/rs7242 genotype combination (n) | | | |
|---|---|---|---|---|
| | IRGC (258) | MRGC (416) | NRGC (73) | P value |
| Age (years) | 50.9/65.9/74.8 | 49.4/63.5/75.2 | 49.4/66.9/76.3 | 0.7743 |
| Sex | 56.6 | 55.3 | 56.2 | 0.9451 |
| (% male) | | | | |
| Surgical (%) | 30.6 | 31.5 | 19.4 | 0.1238 |
| APACHE II | 18.0/24.0/29.0 | 20.0/24.0/29.0 | 18.0/23.0/31.0 | 0.9882 |

TABLE 41
Baseline Characteristics for all placebo-treated PROWESS
subjects with APACHE ≧25 by PROC 2069912 and SERPINE
1 rs7242 combined genotype. 25^{th} percentile, median and
75^{th} percentile values are given for age and APACHE II

| | rs2069912/rs7242 genotype combination (n) | | | |
|---|---|---|---|---|
| | IRGC (138) | MRGC (178) | NRGC (38) | P value |
| Age (years) | 55.1/68.9/75.5 | 55.6/67.3/76.0 | 55.9/70.5/74.6 | 0.8268 |
| Sex | 61.6 | 56.7 | 65.8 | 0.4887 |
| (% male) | | | | |
| Surgical (%) | 28.3 | 27.5 | 23.7 | 0.854 |
| APACHE II | 28.0/31.0/36.0 | 27.0/30.0/34.0 | 27.0/30.0/33.0 | 0.4472 |

TABLE 42
Baseline Characteristics for all XIGRIS ™-treated PROWESS
subjects with APACHE ≧25 by PROC 2069912 and SERPINE 1
rs7242 combined genotype. 25^{th} percentile, median and
75^{th} percentile values are given for age and APACHE II

| | rs2069912/rs7242 genotype combination (n) | | | |
|---|---|---|---|---|
| | IRGC (124) | MRGC (204) | NRGC (33) | P value |
| Age (years) | 57.1/67.9/74.7 | 53.6/67.8/76.1 | 53.7/68.5/75.9 | 0.8356 |
| Sex | 58.1 | 58.3 | 51.5 | 0.7582 |
| (% male) | | | | |
| Surgical (%) | 25.4 | 27.6 | 18.8 | 0.5647 |
| APACHE II | 27.0/30.0/33.0 | 26.0/29.0/33.0 | 29.0/32.0/33.0 | 0.1195 |

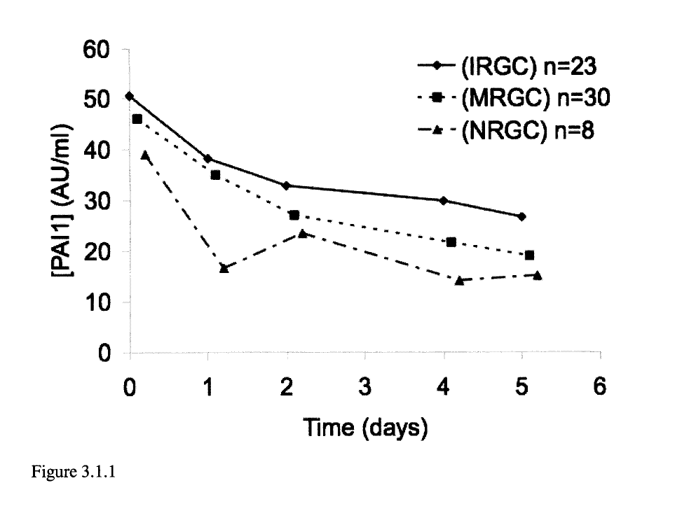

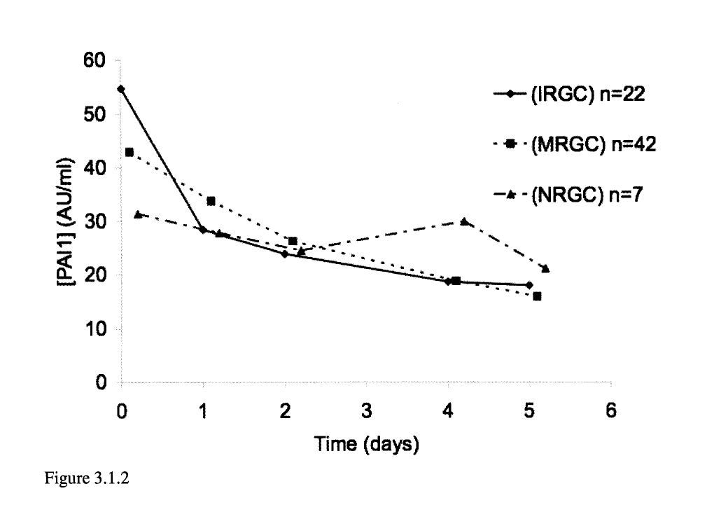

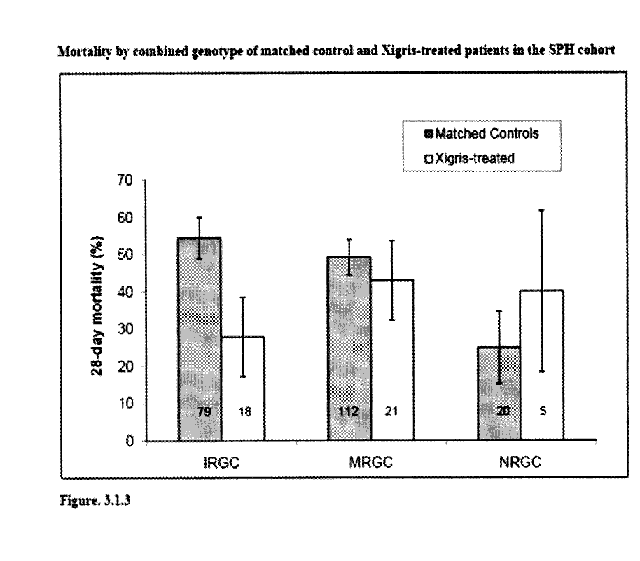

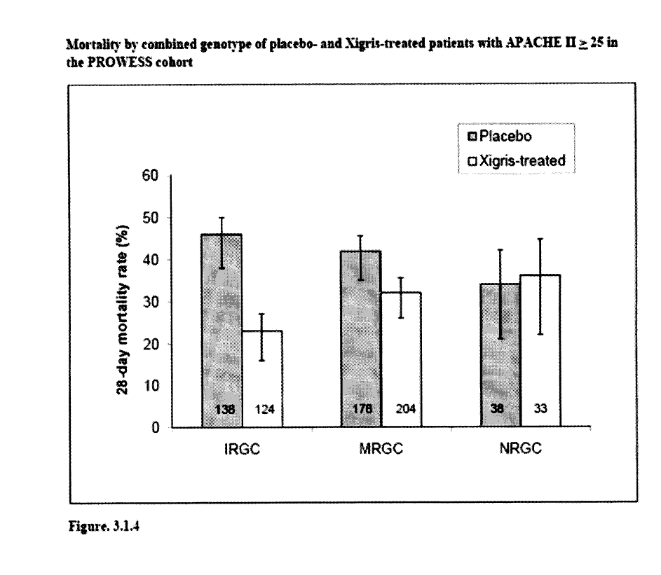

TABLES 43 and 44 show survival data for Placebo and XIGRIS™-treated subjects in the PROWESS severe sepsis cohort by combined PROC rs2069912 and SERPINE1 rs7242 genotype in all subjects with APACHE II ≧25, all subjects and all subjects with two or more organ dysfunctions (MOD ≧2). Subjects who have one of the PROC rs2069912 CC/CT genotypes and one of the SERPINE1 rs7242 GT/TT genotypes are defined as belonging to the Improved Response Genotype Combination (IRGC). Other subjects are classified having the non-IRGC. The non-IRGC group is further subdivided into subjects with a Non Response Genotype Combination (NRGC), with the remainder classified as the Mixed Response Genotype Combination (MRGC). NRGC subjects have both the PROC rs2069912 TT and SERPINE1 rs7242 GG genotype. TABLE 45 shows the logistic regression results modeled IRGC and non-IRGC subjects. When treated with XIGRIS™, subjects with the PROC/SERPINE1 IRGC have improved survival compared to subjects who do not (p=0.0311). FIG. 3.1.3 is a graphical representation of the data in Tables 43 and 44 comparing XIGRIS™ treated and Placebo-treated subjects (all having APACHE II ≧25) by genotype combination, and is expressed as 28-day mortality. Placebo-treated IRGC subjects had a higher mortality rate than the MRGC subjects, which was higher than the NRGC subjects, although this was not statistically significant. It was the IRGC subjects that had the largest reduction in 28-day mortality when treated using XIGRIS™, 23.1% (p=0.0001. This compares to a 9.2% mortality reduction after XIGRIS™ treatment in MRGC subjects (p=0.07) and no mortality reduction in the NRGC subjects (2.2% increase, p=0.78). The interaction statistic testing for a differential response to XIGRIS™ by genotype combination was p=0.06.

TABLE 43
28-day survival by PROC 2069912 and SERPINE 1 rs7242
combined genotype for all Placebo-treated subjects with
APACHE II ≧ 25, All subjects and subjects with two or more
organ dysfunctions (MOD ≧ 2) in PROWESS Severe
Sepsis Cohort. Data is presented as N-survived/N-total
(% Survived)

| Genotype | | | |
|---|---|---|---|
| Combination | Cohort | | |
| Group | APACHE II ≧ 25 | All subjects | MOD ≧ 2 |
| IRGC | 74/138 (54) | 188/276 (68) | 135/204 (66) |
| MRGC | 104/178 (58) | 279/392 (71) | 203/299 (68) |
| NRGC | 25/38 (66) | 56/77 (73) | 40/59 (68) |

TABLE 44
28-day survival by PROC 2069912 and SERPINE 1 rs7242
combined genotype for all XIGRIS ™-treated subjects with
APACHE II ≧ 25, All subjects and subjects with two or more
organ dysfunctions (MOD ≧ 2) in PROWESS Severe
Sepsis Cohort. Data is presented as N-survived/N-total
(% Survived)

| Genotype | | | |
|---|---|---|---|
| Combination | | | |
| Group | APACHE II ≧ 25 | All subjects | MOD ≧ 2 |
| IRGC | 95/124 (77) | 203/258 (79) | 148/192 (77) |
| MRGC | 138/204 (68) | 315/416 (76) | 234/314 (75) |
| NRGC | 21/33 (64) | 52/73 (71) | 35/52 (67) |

TABLE 45
Logistic regression statistics for response to XIGRIS ™ by
PROC rs2069912 CC/CT and SERPINE1 7242 GT/TT combined
genotype (IRGC) in all subjects with APACHE II ≧ 25 in
PROWESS Severe Sepsis Cohort

| | Estimate of | | | |
|---|---|---|---|---|
| | Log Odds | St. Error | P value | model (base) |
| IRGC* Treatment | 0.7231 | 0.3354 | 0.0311 | Non IRGC |

3.1.2 Risk of Death and Response to XIGRIS™ by SERPINE1 rs7242 and PROC rs2069912 Genotype Combination in the SPH Severe Sepsis Cohort

TABLES 46 and 47 show survival data for Control and XIGRIS™-treated subjects in the SPH severe sepsis cohort by combined PROC rs2069912 and SERPINE1 rs7242 genotype. TABLE 48 shows the logistic regression results modeled using the PROC/SERPINE1 IRGC from these two tables. In the absence of XIGRIS™ treatment, subjects with the PROC/SERPINE1 IRGC are predicted to have decreased survival compared to subjects with the non-IRGC (p=0.024). In contrast, when administered XIGRIS™, IRGC subjects are observed to have a trend towards improved survival over those with the PROC rs2069912 TT and SERPINE1 rs7242 GG genotype combination (p=0.126). The results in TABLES 46 and 47 are shown graphically in FIG. 3.1.4, and are expressed in terms of 28-day mortality by combined genotype.

TABLE 46
28-day survival for SPH Control subjects by combined PROC
rs2069912 and SERPINE1 rs7242 genotype. Data is presented
as N-survived/N-total (% Survived)

| Genotype combination | |
|---|---|
| Group | 28-day survival rate |
| IRGC | 36/79 (46) |
| MRGC | 57/112 (51) |
| NRGC | 15/20 (75) |

TABLE 47
28-day survival for SPH XIGRIS ™-treated subjects by
combined PROC rs2069912 and SERPINE1 rs7242 genotype.
Data is presented as N-survived/N-total (% Survived)

| Genotype combination | |
|---|---|
| group | 28-day survival rate |
| IRGC | 13/18 (72) |
| MRGC | 12/21 (57) |
| NRGC | 3/5 (60) |

TABLE 48
Logistic regression statistics for response to XIGRIS ™ by
PROC rs2069912 CC/CT and SERPINE1 7242 GT/TT combined
genotype for all subjects in SPH Severe Sepsis Cohort

| | Estimate of Log Odds | St. Error | P value | model (base) |
|---|---|---|---|---|
| IRGC | −1.276 | 0.564 | 0.024 | NRGC |
| Treatment | −0.693 | 1.049 | 0.509 | NRGC |
| IRGC* | 1.826 | 1.195 | 0.126 | NRGC |
| Treatment | | | | |

FIG. 3.1.1 and FIG. 3.1.2 show PAI-1 levels versus rs7242/rs2069912 genotype combination in PROWESS subjects with APACHE II ≧25 for placebo-treated and XIGRIS™-treated subjects, respectively. In the placebo-treated subjects, the NRGC (i.e. −/−) subjects consistently have the lowest PAI-1 levels, the MRGC (i.e. +/−) subjects consistently have intermediate PAI-1 levels and the IRGC (i.e. +/+) subjects consistently have the highest PAI-1 levels, both pre- and post-infusion. In contrast, while the PAI-1 levels in the XIGRIS™-treated group follow the same pattern at baseline, they change post-infusion. The PAI-1 levels of all subjects are brought to a similar level on days 1 and 2. On days 4 and 5, the PAI-1 levels of the NRGC subjects are higher than those of the MRGC and IRGC individuals.

### Example 4

### Incidence of Adverse Outcomes and Response to XIGRIS™ During the 28-Day Study Period by SERPINE1 rs7242 and PROC rs2069912 Genotypes Alone and in Combination

4.1.1 Incidence of Adverse Outcomes and Response to XIGRIS™ by PROC rs2069912 Genotype for all Subjects in the Prowess Severe Sepsis Cohort

TABLE 49 shows the adverse and serious adverse events for all PROWESS placebo- and XIGRIS™-treated subjects genotyped for PROC rs2069912. An increase in serious adverse events is observed in the TT XIGRIS™-treated group (13.3%) vs. the TT placebo group (9.8%). In contrast an increase in serious adverse events is observed in the CC/CT placebo group (13.6%) vs. the CC/CT XIGRIS™-treated group (10.7%). Serious adverse thrombotic events are similar in both the TT placebo group (2.8%) and the TT XIGRIS™-treated group (2.5%). In contrast an increase in serious adverse thrombotic events is observed in the CC/CT placebo group (3.2%) vs. the CC/CT XIGRIS™-treated group (1.4%).

TABLE 49
Incidence of adverse and serious adverse events by genotype of PROC rs2069912 in
placebo and treated (XIGRIS ™) subjects in the PROWESS Severe Sepsis Cohort
(data is presented as # of events/individuals (fraction)).

| | | Placebo | | | Xigris | | | Placebo | Xigris |
|---|---|---|---|---|---|---|---|---|---|
| | variable | CC | CT | TT | CC | CT | TT | CC/CT | CC/CT |
| AE | ANY | 43/45 | 296/301 | 417/427 | 60/63 | 280/283 | 423/436 | 339/346 | 340/346 |
| | | (0.956) | (0.983) | (0.977) | (0.952) | (0.989) | (0.97) | (0.98) | (0.983) |
| | BLEED | 13/45 | 60/301 | 79/427 | 12/63 | 80/283 | 107/436 | 73/346 | 92/346 |
| | | (0.289) | (0.199) | (0.185) | (0.19) | (0.283) | (0.245) | (0.211) | (0.266) |
| | THROMBOTIC | 5/45 | 20/301 | 31/427 | 2/63 | 14/283 | 38/436 | 25/346 | 16/346 |
| | | (0.111) | (0.066) | (0.073) | (0.032) | (0.049) | (0.087) | (0.072) | (0.046) |
| SAE | ANY | 7/45 | 40/301 | 42/427 | 5/63 | 32/283 | 58/436 | 47/346 | 37/346 |
| | | (0.156) | (0.133) | (0.098) | (0.079) | (0.113) | (0.133) | (0.136) | (0.107) |
| | BLEED | 1/45 | 5/301 | 5/427 | 0/63 | 10/283 | 16/436 | 6/346 | 10/346 |
| | | (0.022) | (0.017) | (0.012) | (0) | (0.035) | (0.037) | (0.017) | (0.029) |
| | THROMBOTIC | 3/45 | 8/301 | 12/427 | 0/63 | 5/283 | 11/436 | 11/346 | 5/346 |
| | | (0.067) | (0.027) | (0.028) | (0) | (0.018) | (0.025) | (0.032) | (0.014) |

Note.
( ) = fraction

TABLE 50 shows logistic regression statistics comparing frequency of adverse and serious adverse events with response to XIGRIS™ by PROC rs2069912 genotypes in all PROWESS placebo- and XIGRIS™-treated subjects. Without treatment the TT group showed a trend for less adverse bleeding events relative to the CC group (p=0.098). With treatment the CT group showed significantly more adverse bleeding events relative to the CC group (p=0.044). With treatment the TT group showed a trend for more adverse bleeding events relative to the CC group (p=0.064). With treatment the TT group showed a trend for more adverse thrombotic events relative to the CC group (p=0.087). With treatment the TT group showed a trend for more serious adverse events relative to the CC group (p=0.094). With treatment the CC/CT group showed a trend for decreased serious adverse events relative to the TT group (p=0.054).

TABLE 50
Logistic Regression of adverse and serious adverse events by genotype + treatment
(XIGRIS ™) of PROC rs2069912 in both a recessive and categorical model for all
subjects in the PROWESS Severe Sepsis Cohort (base: categorical model = CC;
recessive model = TT).

| | | | | Estimate | | |
|---|---|---|---|---|---|---|
| | | | | of Log | Std. | |
| | adverse event | model | term | Odds | Error | P value |
| AE | ANY | categorical | CT | 1.013 | 0.852 | 0.235 |
| | ANY | categorical | TT | 0.662 | 0.791 | 0.402 |
| | ANY | categorical | treatment | −0.072 | 0.934 | 0.938 |
| | ANY | categorical | CT*treatment | 0.528 | 1.189 | 0.657 |
| | ANY | categorical | TT*treatment | −0.176 | 1.027 | 0.864 |
| | ANY | recessive | CC/CT | 0.15 | 0.498 | 0.764 |
| | ANY | recessive | treatment | −0.248 | 0.426 | 0.561 |
| | ANY | recessive | CC/CT*treatment | 0.405 | 0.705 | 0.566 |
| | BLEED | categorical | CT | −0.49 | 0.359 | 0.173 |
| | BLEED | categorical | TT | −0.582 | 0.352 | 0.098 |
| | BLEED | categorical | treatment | −0.546 | 0.459 | 0.235 |
| | BLEED | categorical | CT*treatment | 1.005 | 0.499 | 0.044 |
| | BLEED | categorical | TT*treatment | 0.906 | 0.489 | 0.064 |
| | BLEED | recessive | CC/CT | 0.164 | 0.181 | 0.367 |
| | BLEED | recessive | treatment | 0.36 | 0.167 | 0.031 |
| | BLEED | recessive | CC/CT*treatment | −0.056 | 0.245 | 0.819 |
| | THROMBOTIC | categorical | CT | −0.563 | 0.528 | 0.286 |
| | THROMBOTIC | categorical | TT | −0.468 | 0.51 | 0.359 |
| | THROMBOTIC | categorical | treatment | −1.338 | 0.861 | 0.12 |
| | THROMBOTIC | categorical | CT*treatment | 1.025 | 0.933 | 0.272 |
| | THROMBOTIC | categorical | TT*treatment | 1.537 | 0.897 | 0.087 |
| | THROMBOTIC | recessive | CC/CT | −0.005 | 0.279 | 0.985 |
| | THROMBOTIC | recessive | treatment | 0.199 | 0.252 | 0.431 |
| | THROMBOTIC | recessive | CC/CT*treatment | −0.672 | 0.415 | 0.105 |
| SAE | ANY | categorical | CT | −0.184 | 0.445 | 0.679 |
| | ANY | categorical | TT | −0.524 | 0.442 | 0.236 |
| | ANY | categorical | treatment | −0.759 | 0.622 | 0.222 |
| | ANY | categorical | CT*treatment | 0.575 | 0.671 | 0.391 |
| | ANY | categorical | TT*treatment | 1.1 | 0.658 | 0.094 |
| | ANY | recessive | CC/CT | 0.365 | 0.226 | 0.106 |
| | ANY | recessive | treatment | 0.341 | 0.215 | 0.113 |
| | ANY | recessive | CC/CT*treatment | −0.613 | 0.318 | 0.054 |
| SAE | BLEED | categorical | CT | −0.297 | 1.107 | 0.789 |
| | BLEED | categorical | TT | −0.651 | 1.107 | 0.556 |
| | BLEED* | categorical | treatment | −15.072 | 950.259 | 0.987 |
| | BLEED* | categorical | CT*treatment | 15.846 | 950.259 | 0.987 |
| | BLEED* | categorical | TT*treatment | 16.24 | 950.259 | 0.986 |
| | BLEED | recessive | CC/CT | 0.398 | 0.61 | 0.514 |
| | BLEED | recessive | treatment | 1.168 | 0.517 | 0.024 |
| | BLEED | recessive | CC/CT*treatment | −0.645 | 0.735 | 0.38 |
| | THROMBOTIC | categorical | CT | −0.962 | 0.697 | 0.168 |
| | THROMBOTIC | categorical | TT | −0.904 | 0.665 | 0.174 |
| | THROMBOTIC* | categorical | treatment | −16.218 | 950.258 | 0.986 |
| | THROMBOTIC* | categorical | CT*treatment | 15.8 | 950.259 | 0.987 |
| | THROMBOTIC* | categorical | TT*treatment | 16.107 | 950.258 | 0.986 |
| | THROMBOTIC | recessive | CC/CT | 0.127 | 0.424 | 0.764 |
| | THROMBOTIC | recessive | treatment | −0.111 | 0.423 | 0.793 |
| | THROMBOTIC | recessive | CC/CT*treatment | −0.695 | 0.69 | 0.313 |

*If one arm of SAE event is too small then the power is too small and the logistic regression results become un-reliable.
Note.
treatment = treatment with XIGRIS ™

TABLE 51 compares adverse and serious adverse events in all PROWESS placebo vs. XIGRIS™-treated subjects by PROC rs2069912 genotype using an exact test approach. The CT group had significantly more adverse bleeding events in the XIGRIS™-treated vs. placebo subjects (p=0.02). The TT group had significantly more adverse bleeding events in the XIGRIS™-treated vs. placebo subjects (p=0.032). The CC group showed a trend for less adverse thrombotic events in the XIGRIS™-treated vs. placebo subjects (p=0.07). The TT group had significantly more serious adverse bleeding events in the XIGRIS™-treated vs. placebo subjects (p=0.025).

TABLE 51
An Exact test of adverse and serious adverse events by genotype
in treatment (XIGRIS ™) of PROC rs2069912 for all subjects
in the PROWESS Severe Sepsis Cohort.

| | variable | group | OddsRatio | P.Value |
|---|---|---|---|---|
| AE | ANY | CC | 0.931 | 1 |
| | ANY | CT | 1.575 | 0.726 |
| | ANY | TT | 0.781 | 0.674 |
| | ANY | CC/CT | 1.17 | 1 |
| | BLEED | CC | 0.582 | 0.255 |
| | BLEED | CT | 1.582 | 0.02 |
| | BLEED | TT | 1.432 | 0.032 |
| | BLEED | CC/CT | 1.354 | 0.108 |
| | THROMBOTIC | CC | 0 | 0.07 |
| | THROMBOTIC | CT | 0.659 | 0.579 |
| | THROMBOTIC | TT | 0.895 | 0.835 |
| | THROMBOTIC | CC/CT | 0.447 | 0.205 |
| SAE | ANY | CC | 0.471 | 0.232 |
| | ANY | CT | 0.832 | 0.529 |
| | ANY | TT | 1.406 | 0.136 |
| | ANY | CC/CT | 0.762 | 0.295 |
| SAE | BLEED | CC | 0 | 0.417 |
| | BLEED | CT | 2.166 | 0.193 |
| | BLEED | TT | 3.211 | 0.025 |
| | BLEED | CC/CT | 1.685 | 0.449 |
| | THROMBOTIC | CC | 0.266 | 0.125 |
| | THROMBOTIC | CT | 0.732 | 0.48 |
| | THROMBOTIC | TT | 1.219 | 0.453 |
| | THROMBOTIC | CC/CT | 0.623 | 0.197 |

4.1.2 Incidence of Adverse Outcomes and Response to XIGRIS™ by PROC rs2069912 Genotype for all Subjects with APACHE II ≧25 in the Prowess Severe Sepsis Cohort

TABLE 52 shows the adverse and serious adverse events for PROWESS placebo- and XIGRIS™-treated subjects with an APACHE II ≧25 genotyped for PROC rs2069912. An increase in serious adverse events is observed in the TT XIGRIS™-treated group (14.5%) vs. the TT placebo group (10.6%). In contrast an increase in serious adverse events is observed in the CC/CT placebo group (17.4%) vs. the CC/CT XIGRIS™-treated group (12.4%). A slight decrease in serious adverse thrombotic events is observed in the TT placebo group (3%) vs. the TT XIGRIS™-treated group (3.9%). In contrast an increase in a serious adverse thrombotic events is observed in the CC/CT placebo group (3.6%) vs. the CC/CT XIGRIS™-treated group (1.8).

TABLE 52
Incidence of adverse and serious adverse events by genotype of PROC
rs2069912 in placebo and treated (XIGRIS ™) subjects with
APACHE II ≧ 25 in the PROWESS Severe Sepsis Cohort (data is
presented as # of events/individuals (fraction)).

| | | Placebo | | | Xigris | | | Placebo | Xigris |
|---|---|---|---|---|---|---|---|---|---|
| | | CC | CT | TT | CC | CT | TT | CC/CT | CC/CT |
| AE | ANY | 19/20 | 147/147 | 193/199 | 31/33 | 136/137 | 204/207 | 166/167 | 167/170 |
| | | (0.95) | (1) | (0.97) | (0.939) | (0.993) | (0.986) | (0.994) | (0.982) |
| | BLEED | 8/20 | 31/147 | 42/199 | 8/33 | 42/137 | 64/207 | 39/167 | 50/170 |
| | | (0.4) | (0.211) | (0.211) | (0.242) | (0.307) | (0.309) | (0.234) | (0.294) |
| | THROMBOTIC | 1/20 | 14/147 | 16/199 | 2/33 | 10/137 | 23/207 | 15/167 | 12/170 |
| | | (0.05) | (0.095) | (0.08) | (0.061) | (0.073) | (0.111) | (0.09) | (0.071) |
| SAE | ANY | 2/20 | 27/147 | 21/199 | 2/33 | 19/137 | 30/207 | 29/167 | 21/170 |
| | | (0.1) | (0.184) | (0.106) | (0.061) | (0.139) | (0.145) | (0.174) | (0.124) |
| | BLEED | 0/20 | 2/147 | 2/199 | 0/33 | 7/137 | 7/207 | 2/167 | 7/170 |
| | | (0) | (0.014) | (0.01) | (0) | (0.051) | (0.034) | (0.012) | (0.041) |
| | THROMBOTIC | 0/20 | 6/147 | 6/199 | 0/33 | 3/137 | 8/207 | 6/167 | 3/170 |
| | | (0) | (0.041) | (0.03) | (0) | (0.022) | (0.039) | (0.036) | (0.018) |

TABLE 53 shows logistic regression statistics comparing frequency of adverse and serious adverse events with response to XIGRIS™ by PROC rs2069912 genotypes in PROWESS placebo- and XIGRIS™-treated subjects with an APACHE II ≧25. Within the placebo group the CT group showed a trend for less adverse bleeding events relative to the CC group (p=0.067). Without XIGRIS™ treatment the TT group showed a trend for less adverse bleeding events relative to the CC group (p=0.062). With XIGRIS™ treatment the CT group showed a trend for more adverse bleeding events relative to the CC group (p=0.065). With XIGRIS™ treatment the TT group showed a trend for more adverse bleeding events relative to the CC group (p=0.056). Without XIGRIS™ treatment the CC/CT group showed a trend for more serious adverse events relative to the TT group (p=0.061). With XIGRIS™ treatment the CC/CT group showed a trend for less serious adverse events relative to the TT group (p=0.079).

TABLE 53
Logistic Regression of adverse and serious adverse events by
genotype + treatment (XIGRIS ™) of PROC rs2069912
in both a recessive and categorical model for all subjects with
APACHE II ≧ 25 in the PROWESS Severe Sepsis Cohort
(base: categorical model = CC; recessive model = TT).

| | | | | Estimate | | |
|---|---|---|---|---|---|---|
| | | | | of Log | Std. | |
| | adverse event | model | term | Odds | Error | P value |
| AE | ANY* | categorical | CT | 17.748 | 1557.674 | 0.991 |
| | ANY | categorical | TT | 0.526 | 1.107 | 0.634 |
| | ANY | categorical | treatment | −0.204 | 1.259 | 0.872 |
| | ANY* | categorical | CT*treatment | −15.576 | 1557.675 | 0.992 |
| | ANY | categorical | TT*treatment | 0.952 | 1.447 | 0.511 |
| | ANY | recessive | CC/CT | 1.641 | 1.085 | 0.131 |
| | ANY | recessive | treatment | 0.749 | 0.714 | 0.295 |
| | ANY | recessive | CC/CT*treatment | −1.841 | 1.362 | 0.176 |
| | BLEED | categorical | CT | −0.914 | 0.499 | 0.067 |
| | BLEED | categorical | TT | −0.913 | 0.488 | 0.062 |
| | BLEED | categorical | treatment | −0.734 | 0.611 | 0.23 |
| | BLEED | categorical | CT*treatment | 1.237 | 0.67 | 0.065 |
| | BLEED | categorical | TT*treatment | 1.249 | 0.653 | 0.056 |
| | BLEED | recessive | CC/CT | 0.13 | 0.252 | 0.606 |
| | BLEED | recessive | treatment | 0.515 | 0.23 | 0.025 |
| | BLEED | recessive | CC/CT*treatment | −0.202 | 0.339 | 0.551 |
| | THROMBOTIC | categorical | CT | 0.693 | 1.064 | 0.515 |
| | THROMBOTIC | categorical | TT | 0.508 | 1.059 | 0.632 |
| | THROMBOTIC | categorical | treatment | 0.204 | 1.259 | 0.872 |
| | THROMBOTIC | categorical | CT*treatment | −0.494 | 1.331 | 0.711 |
| | THROMBOTIC | categorical | TT*treatment | 0.154 | 1.305 | 0.906 |
| | THROMBOTIC | recessive | CC/CT | 0.121 | 0.376 | 0.747 |
| | THROMBOTIC | recessive | treatment | 0.357 | 0.342 | 0.296 |
| | THROMBOTIC | recessive | CC/CT*treatment | −0.619 | 0.529 | 0.242 |
| SAE | ANY | categorical | CT | 0.706 | 0.775 | 0.363 |
| | ANY | categorical | TT | 0.06 | 0.78 | 0.939 |
| | ANY | categorical | treatment | −0.544 | 1.043 | 0.602 |
| | ANY | categorical | CT*treatment | 0.209 | 1.093 | 0.848 |
| | ANY | categorical | TT*treatment | 0.906 | 1.086 | 0.404 |
| | ANY | recessive | CC/CT | 0.577 | 0.308 | 0.061 |
| | ANY | recessive | treatment | 0.362 | 0.304 | 0.233 |
| | ANY | recessive | CC/CT*treatment | −0.762 | 0.434 | 0.079 |
| | BLEED* | categorical | CT | 14.468 | 1600.411 | 0.993 |
| | BLEED* | categorical | TT | 14.162 | 1600.411 | 0.993 |
| | BLEED* | categorical | treatment | −0.476 | 2249.652 | 1 |
| | BLEED* | categorical | CT*treatment | 1.838 | 2249.652 | 0.999 |
| | BLEED* | categorical | TT*treatment | 1.714 | 2249.652 | 0.999 |
| | BLEED | recessive | CC/CT | 0.177 | 1.006 | 0.86 |
| | BLEED | recessive | treatment | 1.238 | 0.808 | 0.126 |
| | BLEED | recessive | CC/CT*treatment | 0.027 | 1.144 | 0.981 |
| | THROMBOTIC* | categorical | CT | 15.595 | 1600.411 | 0.992 |
| | THROMBOTIC* | categorical | TT | 15.281 | 1600.411 | 0.992 |
| | THROMBOTIC* | categorical | treatment | −0.476 | 2249.652 | 1 |
| | THROMBOTIC* | categorical | CT*treatment | −0.166 | 2249.652 | 1 |
| | THROMBOTIC* | categorical | TT*treatment | 0.733 | 2249.652 | 1 |
| | THROMBOTIC | recessive | CC/CT | 0.181 | 0.587 | 0.758 |
| | THROMBOTIC | recessive | treatment | 0.257 | 0.549 | 0.64 |
| | THROMBOTIC | recessive | CC/CT*treatment | −0.987 | 0.902 | 0.274 |

*If one arm of SAE event is too small then the power is too small and the logistic regression results become un-reliable.
Note.
treatment = treatment with XIGRIS ™

TABLE 54 compares adverse and serious advents events in PROWESS APACHE II ≧25 placebo vs. XIGRIS™-treated subjects by PROC rs2069912 genotype using an exact test approach. The CT group showed a trend for more adverse bleeding events in the XIGRIS™-treated vs. placebo subjects (p=0.077). The TT group had significantly more adverse bleeding events in the XIGRIS™-treated vs. placebo subjects (p=0.032). The CT group had a trend for more adverse thrombotic events in the XIGRIS™-treated vs. placebo subjects (p=0.094).

TABLE 54
An Exact test of adverse and serious adverse events by genotype
against treatment (XIGRIS ™) of PROC rs2069912 for all subjects
with APACHE II ≧ 25 in the PROWESS Severe Sepsis Cohort.

| | variable | group | OddsRatio | P.Value |
|---|---|---|---|---|
| AE | ANY | CC | 0.819 | 1 |
| | ANY | CT | 0 | 0.482 |
| | ANY | TT | 2.11 | 0.33 |
| | ANY | CC/CT | 0.336 | 0.623 |
| | BLEED | CC | 0.487 | 0.355 |
| | BLEED | CT | 1.651 | 0.077 |
| | BLEED | TT | 1.671 | 0.032 |
| | BLEED | CC/CT | 1.366 | 0.219 |
| | THROMBOTIC | CC | 1.221 | 1 |
| | THROMBOTIC | CT | 0.749 | 0.53 |
| | THROMBOTIC | TT | 1.428 | 0.316 |
| | THROMBOTIC | CC/CT | 0.77 | 0.552 |
| SAE | ANY | CC | 0.587 | 0.627 |
| | ANY | CT | 0.716 | 0.336 |
| | ANY | TT | 1.435 | 0.294 |
| | ANY | CC/CT | 0.671 | 0.222 |
| | BLEED | CC | 0 | 1 |
| | BLEED | CT | 3.887 | 0.094 |
| | BLEED | TT | 3.438 | 0.176 |
| | BLEED | CC/CT | 3.531 | 0.174 |
| | THROMBOTIC | CC | 0 | 1 |
| | THROMBOTIC | CT | 0.527 | 0.503 |
| | THROMBOTIC | TT | 1.292 | 0.787 |
| | THROMBOTIC | CC/CT | 0.483 | 0.334 |

4.1.3 Incidence of Adverse Outcomes and Response to XIGRIS™ by PROC rs2069912 Genotype for all Subjects with Two or More Organ Dysfunctions (MOD ≧2) in the PROWESS Severe Sepsis Cohort

TABLE 55 shows the adverse and serious adverse events for all PROWESS placebo- and XIGRIS™-treated subjects with two or more organ dysfunctions (MOD ≧2) genotyped for PROC rs2069912. An increase in serious adverse events is observed in the TT XIGRIS™-treated group (13%) vs. the TT placebo group (10.7%). In contrast an increase in serious adverse events is observed in the CC/CT placebo group (12.4%) vs. the CC/CT XIGRIS™-treated group (9.1%). Serious adverse thrombotic events are similar in both the TT placebo group (3.4%) and the TT XIGRIS™-treated group (2.7%). In contrast an increase in serious adverse thrombotic events is observed in the CC/CT placebo group (3.1%) vs. the CC/CT XIGRIS™-treated group (1.2%).

TABLE 55
Incidence of adverse and serious adverse events by genotype of
PROC rs2069912 in placebo and treated (XIGRIS ™)
subjects with two or more organ dysfunctions (MOD ≧ 2) in the
PROWESS Severe Sepsis Cohort (data is presented as # of
events/individuals (fraction)).

| | | Placebo | | | Xigris | | | Placebo | Xigris |
|---|---|---|---|---|---|---|---|---|---|
| | | CC | CT | TT | CC | CT | TT | CC/CT | CC/CT |
| AE | ANY | 30\|32 | 222\|226 | 320\|328 | 46\|49 | 202\|204 | 323\|332 | 252\|258 | 248\|253 |
| | | (0.938) | (0.982) | (0.976) | (0.939) | (0.99) | (0.973) | (0.977) | (0.98) |
| | BLEED | 10\|32 | 45\|226 | 60\|328 | 11\|49 | 60\|204 | 82\|332 | 55\|258 | 71\|253 |
| | | (0.312) | (0.199) | (0.183) | (0.224) | (0.294) | (0.247) | (0.213) | (0.281) |
| | THROMBOTIC | 3\|32 | 16\|226 | 23\|328 | 1\|49 | 10\|204 | 29\|332 | 19\|258 | 11\|253 |
| | | (0.094) | (0.071) | (0.07) | (0.02) | (0.049) | (0.087) | (0.074) | (0.043) |
| SAE | ANY | 4\|32 | 28\|226 | 35\|328 | 5\|49 | 18\|204 | 43\|332 | 32\|258 | 23\|253 |
| | | (0.125) | (0.124) | (0.107) | (0.102) | (0.088) | (0.13) | (0.124) | (0.091) |
| | BLEED | 1\|32 | 4\|226 | 5\|328 | 0\|49 (0) | 10\|204 | 11\|332 | 5\|258 | 10\|253 |
| | | (0.031) | (0.018) | (0.015) | | (0.049) | (0.033) | (0.019) | (0.04) |
| | THROMBOTIC | 1\|32 | 7\|226 | 11\|328 | 0\|49 (0) | 3\|204 | 9\|332 | 8\|258 | 3\|253 |
| | | (0.031) | (0.031) | (0.034) | | (0.015) | (0.027) | (0.031) | (0.012) |

TABLE 56 shows logistic regression statistics comparing frequency of adverse and serious adverse events with response to XIGRIS™ by PROC rs2069912 genotypes in PROWESS placebo- and XIGRIS™-treated subjects with two or more organ dysfunctions (MOD ≧2). Within the placebo group the TT group showed a trend for less adverse bleeding events relative to the CC group (p=0.082). With XIGRIS™ treatment the CT group showed a trend for more adverse bleeding events relative to the CC group (p=0.084).

TABLE 56
Logistic Regression of adverse and serious adverse events by
genotype + treatment (XIGRIS ™) of PROC rs2069912
in both a recessive and categorical model for all subjects with
two or more organ dysfunctions (MOD ≧ 2) in the PROWESS
Severe Sepsis Cohort (base: categorical model = CC; recessive
model = TT).

| | | | | Estimate | | |
|---|---|---|---|---|---|---|
| | | | | of Log | Std. | |
| | adverse event | model | term | Odds | Error | P value |
| AE | ANY | categorical | CT | 1.308 | 0.888 | 0.14 |
| | ANY | categorical | TT | 0.981 | 0.813 | 0.228 |
| | ANY | categorical | treatment | 0.022 | 0.943 | 0.981 |
| | ANY | categorical | CT*treatment | 0.577 | 1.284 | 0.653 |
| | ANY | categorical | TT*treatment | −0.13 | 1.063 | 0.902 |
| | ANY | recessive | CC/CT | 0.049 | 0.547 | 0.929 |
| | ANY | recessive | treatment | −0.108 | 0.492 | 0.826 |
| | ANY | recessive | CC/CT*treatment | 0.275 | 0.785 | 0.726 |
| | BLEED | categorical | CT | −0.603 | 0.416 | 0.147 |
| | BLEED | categorical | TT | −0.708 | 0.407 | 0.082 |
| | BLEED | categorical | treatment | −0.451 | 0.513 | 0.379 |
| | BLEED | categorical | CT*treatment | 0.968 | 0.56 | 0.084 |
| | BLEED | categorical | TT*treatment | 0.833 | 0.547 | 0.128 |
| | BLEED | recessive | CC/CT | 0.191 | 0.209 | 0.36 |
| | BLEED | recessive | treatment | 0.382 | 0.191 | 0.046 |
| | BLEED | recessive | CC/CT*treatment | −0.017 | 0.282 | 0.951 |
| | THROMBOTIC | categorical | CT | −0.306 | 0.66 | 0.643 |
| | THROMBOTIC | categorical | TT | −0.316 | 0.644 | 0.623 |
| | THIROMBOTIC | categorical | treatment | −1.603 | 1.178 | 0.174 |
| | THROMBOTIC | categorical | CT*treatment | 1.212 | 1.249 | 0.332 |
| | THROMBOTIC | categorical | TT*treatment | 1.841 | 1.214 | 0.129 |
| | THROMBOTIC | recessive | CC/CT | 0.053 | 0.322 | 0.87 |
| | THROMBOTIC | recessive | treatment | 0.238 | 0.291 | 0.412 |
| | THROMBOTIC | recessive | CC/CT*treatment | −0.797 | 0.486 | 0.101 |
| SAE | ANY | categorical | CT | −0.01 | 0.571 | 0.986 |
| | ANY | categorical | TT | −0.179 | 0.564 | 0.751 |
| | ANY | categorical | treatment | −0.229 | 0.713 | 0.748 |
| | ANY | categorical | CT*treatment | −0.15 | 0.781 | 0.847 |
| | ANY | categorical | TT*treatment | 0.448 | 0.753 | 0.552 |
| | ANY | recessive | CC/CT | 0.17 | 0.26 | 0.513 |
| | ANY | recessive | treatment | 0.22 | 0.242 | 0.365 |
| | ANY | recessive | CC/CT*treatment | −0.567 | 0.377 | 0.132 |
| | BLEED | categorical | CT | −0.582 | 1.134 | 0.608 |
| | BLEED | categorical | TT | −0.734 | 1.111 | 0.509 |
| | BLEED* | categorical | treatment | −15.177 | 952.992 | 0.987 |
| | BLEED* | categorical | CT*treatment | 16.228 | 952.992 | 0.986 |
| | BLEED* | categorical | TT*treatment | 15.972 | 952.992 | 0.987 |
| | BLEED | recessive | CC/CT | 0.244 | 0.638 | 0.702 |
| | BLEED | recessive | treatment | 0.795 | 0.545 | 0.145 |
| | BLEED | recessive | CC/CT*treatment | −0.061 | 0.778 | 0.937 |
| | THROMBOTIC | categorical | CT | −0.009 | 1.086 | 0.993 |
| | THROMBOTIC | categorical | TT | 0.073 | 1.061 | 0.945 |
| | THROMBOTIC* | categorical | treatment | −15.177 | 952.992 | 0.987 |
| | THROMBOTIC* | categorical | CT*treatment | 14.416 | 952.992 | 0.988 |
| | THROMBOTIC* | categorical | TT*treatment | 14.958 | 952.992 | 0.987 |
| | THROMBOTIC | recessive | CC/CT | −0.081 | 0.472 | 0.864 |
| | THROMBOTIC | recessive | treatment | −0.219 | 0.456 | 0.631 |
| | THROMBOTIC | recessive | CC/CT*treatment | −0.761 | 0.821 | 0.354 |

*If one arm of SAE event is too small then the power is too small and the logistic regression results become un-reliable.
Note.
treatment = treatment with XIGRIS ™

TABLE 57 compares adverse and serious advents events in PROWESS placebo vs. XIGRIS™-treated subjects with two or more organ dysfunctions (MOD ≧2) by PROC rs2069912 genotype using an exact test approach. The CT group had significantly more adverse bleeding events in the XIGRIS™-treated vs. placebo subjects (p=0.025). The TT group had significantly more adverse bleeding events in the XIGRIS™-treated vs. placebo subjects (p=0.047). The CC/CT group had a trend for more adverse bleeding events in the XIGRIS™-treated vs. placebo subjects (p=0.082).

TABLE 57
An Exact test of adverse and serious adverse events by genotype
against treatment (XIGRIS ™) of PROC rs2069912 for all subjects
with two or more organ dysfunctions (MOD ≧ 2) in the PROWESS
Severe Sepsis Cohort.

| | variable | group | OddsRatio | P.Value |
|---|---|---|---|---|
| AE | ANY | CC | 1.022 | 1 |
| | ANY | CT | 1.817 | 0.688 |
| | ANY | TT | 0.897 | 1 |
| | ANY | CC/CT | 1.181 | 1 |
| | BLEED | CC | 0.641 | 0.441 |
| | BLEED | CT | 1.674 | 0.025 |
| | BLEED | TT | 1.464 | 0.047 |
| | BLEED | CC/CT | 1.439 | 0.082 |
| | THROMBOTIC | CC | 0.205 | 0.295 |
| | THROMBOTIC | CT | 0.677 | 0.42 |
| | THROMBOTIC | TT | 1.269 | 0.471 |
| | THROMBOTIC | CC/CT | 0.572 | 0.188 |
| SAE | ANY | CC | 0.798 | 0.734 |
| | ANY | CT | 0.685 | 0.275 |
| | ANY | TT | 1.245 | 0.4 |
| | ANY | CC/CT | 0.707 | 0.255 |
| | BLEED | CC | 0 | 0.395 |
| | BLEED | CT | 2.854 | 0.1 |
| | BLEED | TT | 2.211 | 0.205 |
| | BLEED | CC/CT | 2.079 | 0.2 |
| | THROMBOTIC | CC | 0 | 0.395 |
| | THROMBOTIC | CT | 0.468 | 0.345 |
| | THROMBOTIC | TT | 0.803 | 0.657 |
| | THROMBOTIC | CC/CT | 0.376 | 0.222 |

4.2.1 Incidence of Adverse Outcomes and Response to XIGRIS™ by SERPINE1 rs7242 Genotype for all Subjects in the Prowess Severe Sepsis Cohort

TABLE 58 shows the adverse and serious adverse events for all PROWESS placebo- and XIGRIS™-treated subjects genotyped for SERPINE1 rs7242. An increase in serious adverse events is observed in the GG XIGRIS™-treated (15.2%) vs. the GG placebo group (9.2%). In contrast there is very little difference in serious adverse events observed in the TT/GT placebo group (12.1%) vs. the TT/GT XIGRIS™-treated group (11.7%). There is no difference in serious adverse bleeding events in the GG placebo group (2.1%) vs. the GG XIGRIS™-treated (2.1%). In contrast there is a decrease in serious adverse bleeding events observed in the TT/GT placebo group (1.6%) vs. the TT/GT XIGRIS™-treated group (3.8%). A decrease in serious adverse thrombotic events is observed in the GG placebo group (2.1%) vs. the GG XIGRIS™-treated group (3.4%). In contrast an increase in serious adverse thrombotic events is observed in the TT/GT placebo group (3.3%) vs. the TT/GT XIGRIS™-treated group (1.8%).

TABLE 58
Incidence of adverse and serious adverse events by genotype of
SERPINE1 rs7242 in placebo and treated (XIGRIS ™)
subjects in the PROWESS Severe Sepsis Cohort (data is
presented as # of events/individuals (fraction)).

| | | Placebo | | | Xigris | | | Placebo | Xigris |
|---|---|---|---|---|---|---|---|---|---|
| | | GG | GT | TT | GG | GT | TT | TT/GT | TT/GT |
| AE | ANY | 137/141 | 323/331 | 275/280 | 138/145 | 337/343 | 257/263 | 598/611 | 594/606 |
| | | (0.972) | (0.976) | (0.982) | (0.952) | (0.983) | (0.977) | (0.979) | (0.98) |
| | BLEED | 24/141 | 58/331 | 68/280 | 36/145 | 84/343 | 70/263 | 126/611 | 154/606 |
| | | (0.17) | (0.175) | (0.243) | (0.248) | (0.245) | (0.266) | (0.206) | (0.254) |
| | THROMBOTIC | 11/141 | 23/331 | 19/280 | 16/145 | 24/343 | 14/263 | 42/611 | 38/606 |
| | | (0.078) | (0.069) | (0.068) | (0.11) | (0.07) | (0.053) | (0.069) | (0.063) |
| SAE | ANY | 13/141 | 40/331 | 34/280 | 22/145 | 40/343 | 31/263 | 74/611 | 71/606 |
| | | (0.092) | (0.121) | (0.121) | (0.152) | (0.117) | (0.118) | (0.121) | (0.117) |
| | BLEED | 3/141 | 4/331 | 6/280 | 3/145 | 12/343 | 11/263 | 10/611 | 23/606 |
| | | (0.021) | (0.012) | (0.021) | (0.021) | (0.035) | (0.042) | (0.016) | (0.038) |
| | THROMBOTIC | 3/141 | 12/331 | 8/280 | 5/145 | 6/343 | 5/263 | 20/611 | 11/606 |
| | | (0.021) | (0.036) | (0.029) | (0.034) | (0.017) | (0.019) | (0.033) | (0.018) |

TABLE 59 shows logistic regression statistics comparing frequency of adverse and serious adverse events with response to XIGRIS™ by SERPINE1 rs7242 genotypes in all PROWESS placebo- and XIGRIS™-treated subjects. Within the placebo group the TT group showed a trend for more adverse bleeding events relative to the GG group (p=0.09).

TABLE 59
Logistic Regression of adverse and serious adverse events by
genotype + treatment (XIGRIS ™) of SERPINE1 rs7242
in both a recessive and categorical model for all subjects in the
PROWESS Severe Sepsis Cohort (base: categorical and recessive
model = GG).

| | | | | Estimate | | |
|---|---|---|---|---|---|---|
| | | | | of Log | Std. | |
| | adverse event | model | term | Odds | Error | P value |
| AE | ANY | categorical | GT | 0.165 | 0.621 | 0.791 |
| | ANY | categorical | TT | 0.474 | 0.679 | 0.485 |
| | ANY | categorical | treatment | −0.552 | 0.638 | 0.387 |
| | ANY | categorical | GT*treatment | 0.882 | 0.84 | 0.293 |
| | ANY | categorical | TT*treatment | 0.302 | 0.884 | 0.732 |
| | ANY | recessive | TT/GT | 0.295 | 0.58 | 0.611 |
| | ANY | recessive | treatment | −0.552 | 0.638 | 0.387 |
| | ANY | recessive | TT/GT*treatment | 0.626 | 0.756 | 0.408 |
| | BLEED | categorical | GT | 0.035 | 0.267 | 0.895 |
| | BLEED | categorical | TT | 0.447 | 0.264 | 0.09 |
| | BLEED | categorical | treatment | 0.476 | 0.295 | 0.107 |
| | BLEED | categorical | GT*treatment | −0.053 | 0.352 | 0.88 |
| | BLEED | categorical | TT*treatment | −0.353 | 0.355 | 0.32 |
| | BLEED | recessive | TT/GT | 0.236 | 0.245 | 0.336 |
| | BLEED | recessive | treatment | 0.476 | 0.295 | 0.107 |
| | BLEED | recessive | TT/GT*treatment | −0.205 | 0.325 | 0.528 |
| | THROMBOTIC | categorical | GT | −0.125 | 0.381 | 0.743 |
| | THROMBOTIC | categorical | TT | −0.15 | 0.394 | 0.702 |
| | THROMBOTIC | categorical | treatment | 0.382 | 0.411 | 0.352 |
| | THROMBOTIC | categorical | GT*treatment | −0.375 | 0.51 | 0.462 |
| | THROMBOTIC | categorical | TT*treatment | −0.641 | 0.548 | 0.243 |
| | THROMBOTIC | recessive | TT/GT | −0.137 | 0.352 | 0.698 |
| | THROMBOTIC | recessive | treatment | 0.382 | 0.411 | 0.352 |
| | THROMBOTIC | recessive | TT/GT*treatment | −0.481 | 0.472 | 0.308 |
| SAE | ANY | categorical | GT | 0.303 | 0.336 | 0.368 |
| | ANY | categorical | TT | 0.308 | 0.344 | 0.37 |
| | ANY | categorical | treatment | 0.566 | 0.372 | 0.128 |
| | ANY | categorical | GT*treatment | −0.606 | 0.442 | 0.17 |
| | ANY | categorical | TT*treatment | −0.6 | 0.456 | 0.189 |
| | ANY | recessive | TT/GT | 0.305 | 0.316 | 0.335 |
| | ANY | recessive | treatment | 0.566 | 0.372 | 0.128 |
| | ANY | recessive | TT/GT*treatment | −0.604 | 0.412 | 0.143 |
| | BLEED | categorical | GT | −0.575 | 0.77 | 0.455 |
| | BLEED | categorical | TT | 0.007 | 0.715 | 0.992 |
| | BLEED | categorical | treatment | −0.029 | 0.825 | 0.972 |
| | BLEED | categorical | GT*treatment | 1.115 | 1.01 | 0.27 |
| | BLEED | categorical | TT*treatment | 0.718 | 0.973 | 0.46 |
| | BLEED | recessive | TT/GT | −0.267 | 0.665 | 0.688 |
| | BLEED | recessive | treatment | −0.029 | 0.825 | 0.972 |
| | BLEED | recessive | TT/GT*treatment | 0.892 | 0.91 | 0.327 |
| | THROMBOTIC | categorical | GT | 0.548 | 0.653 | 0.401 |
| | THROMBOTIC | categorical | TT | 0.302 | 0.685 | 0.659 |
| | THROMBOTIC | categorical | treatment | 0.496 | 0.74 | 0.502 |
| | THROMBOTIC | categorical | GT*treatment | −1.244 | 0.897 | 0.165 |
| | THROMBOTIC | categorical | TT*treatment | −0.914 | 0.938 | 0.33 |
| | THROMBOTIC | recessive | TT/GT | 0.443 | 0.626 | 0.48 |
| | THROMBOTIC | recessive | treatment | 0.496 | 0.74 | 0.502 |
| | THROMBOTIC | recessive | TT/GT*treatment | −1.101 | 0.832 | 0.186 |

Note.
treatment = treatment with XIGRIS ™

TABLE 60 shows adverse and serious advents events in all PROWESS placebo vs. XIGRIS™-treated subjects by SERPINE1 rs7242 genotype using an exact test approach. The GT group had significantly more adverse bleeding events in the XIGRIS™-treated vs. placebo subjects (p=0.03). The TT/GT group had significantly more adverse bleeding events in the XIGRIS™-treated vs. placebo subjects (p=0.049). The GT group showed a trend for more serious adverse bleeding events in the XIGRIS™-treated vs. placebo subjects (p=0.074). The TT/GT group had significantly more serious adverse bleeding events in the XIGRIS™-treated vs. placebo subjects (p=0.022).

TABLE 60
An Exact test of adverse and serious adverse events by genotype
against treatment (XIGRIS ™) of SERPINE1 rs7242 for all
subjects in the PROWESS Severe Sepsis Cohort.

| | variable | group | OddsRatio | P.Value |
|---|---|---|---|---|
| AE | ANY | GG | 0.577 | 0.541 |
| | ANY | GT | 1.39 | 0.598 |
| | ANY | TT | 0.779 | 0.766 |
| | ANY | TT/GT | 1.076 | 1 |
| | BLEED | GG | 1.607 | 0.112 |
| | BLEED | GT | 1.526 | 0.03 |
| | BLEED | TT | 1.13 | 0.555 |
| | BLEED | TT/GT | 1.311 | 0.049 |
| | THROMBOTIC | GG | 1.464 | 0.42 |
| | THROMBOTIC | GT | 1.007 | 1 |
| | THROMBOTIC | TT | 0.773 | 0.59 |
| | THROMBOTIC | TT/GT | 0.906 | 0.729 |
| SAE | ANY | GG | 1.758 | 0.15 |
| | ANY | GT | 0.96 | 0.905 |
| | ANY | TT | 0.967 | 1 |
| | ANY | TT/GT | 0.963 | 0.86 |
| | BLEED | GG | 0.972 | 1 |
| | BLEED | GT | 2.96 | 0.074 |
| | BLEED | TT | 1.991 | 0.22 |
| | BLEED | TT/GT | 2.369 | 0.022 |
| | THROMBOTIC | GG | 1.64 | 0.723 |
| | THROMBOTIC | GT | 0.474 | 0.155 |
| | THROMBOTIC | TT | 0.659 | 0.579 |
| | THROMBOTIC | TT/GT | 0.547 | 0.145 |

4.2.2 Incidence of Adverse Outcomes and Response to XIGRIS™ by SERPINE1 rs7242 Genotype for all Subjects with APACHE II ≧25 in the Prowess Severe Sepsis Cohort

TABLE 61 shows the adverse and serious adverse events for all PROWESS placebo- and XIGRIS™-treated subjects with an APACHE II ≧25 genotyped for SERPINE1 rs7242. An increase in serious adverse events is observed in the GG XIGRIS™-treated (18.3%) vs. the GG placebo group (7.7%). In contrast there was a slight increase in serious adverse events observed in the TT/GT placebo group (15%) vs. the TT/GT XIGRIS™-treated group (13%). There is very little difference in serious adverse bleeding events in the GG placebo group (1.5%) vs. the GG XIGRIS™-treated (1.4%). In contrast there is a decreases in serious adverse bleeding events observed in the TT/GT placebo group (1.0%) vs. the TT/GT XIGRIS™-treated group (4.8%). A decrease in serious adverse thrombotic events is observed in the GG placebo group (1.5%) vs. the GG XIGRIS™-treated group (5.6%). In contrast an increase in serious adverse thrombotic events is observed in the TT/GT placebo group (3.8%) vs. the TT/GT XIGRIS™-treated group (2.4%).

TABLE 61
Incidence of adverse and serious adverse events by genotype
of SERPINE1 rs7242 in placebo and treated (XIGRIS ™)
subjects with APACHE II ≧ 25 in the PROWESS Severe Sepsis
Cohort (data is presented as # of events/individuals (fraction)).

| | | Placebo | | | Xigris | | | Placebo | Xigris |
|---|---|---|---|---|---|---|---|---|---|
| | | GG | GT | TT | GG | GT | TT | TT/GT | TT/GT |
| AE | ANY | 64/65 | 147/152 | 140/141 | 68/71 | 159/160 | 131/133 | 287/293 | 290/293 |
| | | (0.985) | (0.967) | (0.993) | (0.958) | (0.994) | (0.985) | (0.98) | (0.99) |
| | BLEED | 11/65 | 28/152 | 41/141 | 19/71 | 53/160 | 39/133 | 69/293 | 92/293 |
| | | (0.169) | (0.184) | (0.291) | (0.268) | (0.331) | (0.293) | (0.235) | (0.314) |
| | THROMBOTIC | 6/65 | 13/152 | 11/141 | 12/71 | 15/160 | 8/133 | 24/293 | 23/293 |
| | | (0.092) | (0.086) | (0.078) | (0.169) | (0.094) | (0.06) | (0.082) | (0.078) |
| SAE | ANY | 5/65 | 23/152 | 21/141 | 13/71 | 20/160 | 18/133 | 44/293 | 38/293 |
| | | (0.077) | (0.151) | (0.149) | (0.183) | (0.125) | (0.135) | (0.15) | (0.13) |
| | BLEED | 1/65 | 1/152 | 2/141 | 1/71 | 8/160 | 6/133 | 3/293 | 14/293 |
| | | (0.015) | (0.007) | (0.014) | (0.014) | (0.05) | (0.045) | (0.01) | (0.048) |
| | THROMBOTIC | 1/65 | 6/152 | 5/141 | 4/71 | 3/160 | 4/133 | 11/293 | 7/293 |
| | | (0.015) | (0.039) | (0.035) | (0.056) | (0.019) | (0.03) | (0.038) | (0.024) |

TABLE 58 shows logistic regression statistics comparing frequency of adverse and serious adverse events with response to XIGRIS™ by SERPINE1 rs7242 genotypes in all PROWESS placebo- and XIGRIS™-treated subjects with an APACHE II ≧25. With treatment the GT group showed a trend for more adverse events relative to the GG group (p=0.089). Within the placebo group the TT group showed a trend for more adverse bleeding events relative to the GG group (p=0.065). With XIGRIS™-treatment the GT group showed a trend for less serious adverse events relative to the GG group (p=0.061). With XIGRIS™-treatment the TT group showed a trend for less serious adverse events relative to the GG group (p=0.094). With XIGRIS™-treatment the TT/GT group showed a trend for less serious adverse events relative to the GG group (p=0.056).

TABLE 62
Logistic Regression of adverse and serious adverse events by
genotype + treatment (XIGRIS ™) of SERPINE1 rs7242
in both a recessive and categorical model for all subjects with
APACHE II ≧ 25 in the PROWESS Severe Sepsis Cohort
(base: categorical and recessive model = GG).

| | | | | Estimate | | |
|---|---|---|---|---|---|---|
| | | | | of Log | Std. | |
| | adverse event | model | term | Odds | Error | P value |
| AE | ANY | categorical | GT | −0.778 | 1.106 | 0.482 |
| | ANY | categorical | TT | 0.783 | 1.422 | 0.582 |
| | ANY | categorical | treatment | −1.038 | 1.168 | 0.374 |
| | ANY | categorical | GT*treatment | 2.726 | 1.605 | 0.089 |
| | ANY | categorical | TT*treatment | 0.278 | 1.697 | 0.87 |
| | ANY | recessive | TT/GT | −0.291 | 1.089 | 0.789 |
| | ANY | recessive | treatment | −1.038 | 1.168 | 0.374 |
| | ANY | recessive | TT/GT*treatment | 1.742 | 1.368 | 0.203 |
| | BLEED | categorical | GT | 0.103 | 0.391 | 0.792 |
| | BLEED | categorical | TT | 0.699 | 0.379 | 0.065 |
| | BLEED | categorical | treatment | 0.584 | 0.426 | 0.17 |
| | BLEED | categorical | GT*treatment | 0.201 | 0.503 | 0.689 |
| | BLEED | categorical | TT*treatment | −0.572 | 0.502 | 0.254 |
| | BLEED | recessive | TT/GT | 0.414 | 0.358 | 0.248 |
| | BLEED | recessive | treatment | 0.584 | 0.426 | 0.17 |
| | BLEED | recessive | TT/GT*treatment | −0.188 | 0.465 | 0.685 |
| | THROMBOTIC | categorical | GT | −0.084 | 0.517 | 0.871 |
| | THROMBOTIC | categorical | TT | −0.184 | 0.531 | 0.729 |
| | THROMBOTIC | categorical | treatment | 0.693 | 0.533 | 0.193 |
| | THROMBOTIC | categorical | GT*treatment | −0.592 | 0.665 | 0.373 |
| | THROMBOTIC | categorical | TT*treatment | −0.972 | 0.718 | 0.176 |
| | THROMBOTIC | recessive | TT/GT | −0.131 | 0.479 | 0.784 |
| | THROMBOTIC | recessive | treatment | 0.693 | 0.533 | 0.193 |
| | THROMBOTIC | recessive | TT/GT*treatment | −0.739 | 0.614 | 0.228 |
| SAE | ANY | categorical | GT | 0.761 | 0.518 | 0.142 |
| | ANY | categorical | TT | 0.742 | 0.522 | 0.155 |
| | ANY | categorical | treatment | 0.989 | 0.558 | 0.076 |
| | ANY | categorical | GT*treatment | −1.211 | 0.647 | 0.061 |
| | ANY | categorical | TT*treatment | −1.101 | 0.657 | 0.094 |
| | ANY | recessive | TT/GT | 0.752 | 0.493 | 0.128 |
| | ANY | recessive | treatment | 0.989 | 0.558 | 0.076 |
| | ANY | recessive | TT/GT*treatment | −1.16 | 0.606 | 0.056 |
| | BLEED | categorical | GT | −0.858 | 1.422 | 0.546 |
| | BLEED | categorical | TT | −0.082 | 1.234 | 0.947 |
| | BLEED | categorical | treatment | −0.09 | 1.425 | 0.95 |
| | BLEED | categorical | GT*treatment | 2.162 | 1.78 | 0.224 |
| | BLEED | categorical | TT*treatment | 1.279 | 1.647 | 0.438 |
| | BLEED | recessive | TT/GT | −0.412 | 1.163 | 0.723 |
| | BLEED | recessive | treatment | −0.09 | 1.425 | 0.95 |
| | BLEED | recessive | TT/GT*treatment | 1.669 | 1.563 | 0.286 |
| | THROMBOTIC | categorical | GT | 0.967 | 1.09 | 0.375 |
| | THROMBOTIC | categorical | TT | 0.856 | 1.106 | 0.439 |
| | THROMBOTIC | categorical | treatment | 1.34 | 1.132 | 0.236 |
| | THROMBOTIC | categorical | GT*treatment | −2.106 | 1.339 | 0.116 |
| | THROMBOTIC | categorical | TT*treatment | −1.511 | 1.321 | 0.253 |
| | THROMBOTIC | recessive | TT/GT | 0.915 | 1.054 | 0.385 |
| | THROMBOTIC | recessive | treatment | 1.34 | 1.132 | 0.236 |
| | THROMBOTIC | recessive | TT/GT*treatment | −1.807 | 1.233 | 0.143 |

Note.
treatment = treatment with XIGRIS ™

TABLE 63 compares adverse and serious advents events in PROWESS APACHE II ≧25 placebo vs. XIGRIS™-treated subjects by SERPINE1 rs7242 genotype using an exact test approach. The GT group had significantly more adverse bleeding events in the XIGRIS™-treated vs. placebo subjects (p=0.004). The TT/GT group had significantly more adverse bleeding events in the XIGRIS™-treated vs. placebo subjects (p=0.042). The GG group showed a trend for more serious adverse events in the XIGRIS™-treated vs. placebo subjects (p=0.08). The GT group showed significantly more serious adverse bleeding events in the XIGRIS™-treated vs. placebo subjects (p=0.037). The TT/GT group had significantly more serious adverse bleeding events in the XIGRIS™-treated vs. placebo subjects (p=0.012).

TABLE 63
An Exact test of adverse and serious adverse events by genotype
against treatment (XIGRIS ™) of SERPINE1 rs7242 for all
subjects with APACHE II ≧ 25 in the PROWESS
Severe Sepsis Cohort.

| | variable | group | OddsRatio | P.Value |
|---|---|---|---|---|
| AE | ANY | GG | 0.357 | 0.621 |
| | ANY | GT | 5.383 | 0.113 |
| | ANY | TT | 0.469 | 0.613 |
| | ANY | TT/GT | 2.019 | 0.504 |
| | BLEED | GG | 1.786 | 0.215 |
| | BLEED | GT | 2.188 | 0.004 |
| | BLEED | TT | 1.012 | 1 |
| | BLEED | TT/GT | 1.485 | 0.042 |
| | THROMBOTIC | GG | 1.99 | 0.214 |
| | THROMBOTIC | GT | 1.106 | 0.845 |
| | THROMBOTIC | TT | 0.757 | 0.638 |
| | THROMBOTIC | TT/GT | 0.955 | 1 |
| SAE | ANY | GG | 2.671 | 0.08 |
| | ANY | GT | 0.802 | 0.516 |
| | ANY | TT | 0.895 | 0.863 |
| | ANY | TT/GT | 0.844 | 0.552 |
| | BLEED | GG | 0.915 | 1 |
| | BLEED | GT | 7.907 | 0.037 |
| | BLEED | TT | 3.27 | 0.162 |
| | BLEED | TT/GT | 4.839 | 0.012 |
| | THROMBOTIC | GG | 3.788 | 0.368 |
| | THROMBOTIC | GT | 0.466 | 0.326 |
| | THROMBOTIC | TT | 0.844 | 1 |
| | THROMBOTIC | TT/GT | 0.628 | 0.474 |

4.23 Incidence of Adverse Outcomes and Response to XIGRIS™ by SERPINE1 rs7242 for all Subjects with Two or More Organ Dysfunctions (MOD ≧2) in the Prowess Severe Sepsis Cohort

TABLE 64 shows the adverse and serious adverse events for all PROWESS placebo- and XIGRIS™-treated subjects with two or more organ dysfunctions (MOD ≧2) genotyped for SERPINE1 rs7242. An increase in serious adverse events is observed in the GG XIGRIS™-treated (13.9%) vs. the GG placebo group (9.2%). In contrast there was a slight increase in serious adverse events observed in the TT/GT placebo group (12%) vs. the TT/GT XIGRIS™-treated group (11.1%). A decrease in serious adverse thrombotic events is observed in the GG placebo group (1.8%) vs. the GG XIGRIS™-treated group (4.0%). In contrast an increase in serious adverse thrombotic events is observed in the TT/GT placebo group (3.7%) vs. the TT/GT XIGRIS™-treated group (1.7%).

TABLE 64
Incidence of adverse and serious adverse events by genotype
of SERPINE1 rs7242 in placebo and treated (XIGRIS ™)
subjects with two or more organ dysfunctions (MOD ≧ 2) in the
PROWESS Severe Sepsis Cohort (data is presented as # of
events/individuals (fraction)).

| | | Placebo | | | Xigris | | | Placebo | Xigris |
|---|---|---|---|---|---|---|---|---|---|
| | variable | GG | GT | TT | GG | GT | TT | TT/GT | TT/GT |
| AE | ANY | 105/109 | 248/254 | 201/205 | 95/101 | 250/252 | 202/208 | 449/459 | 452/460 |
| | | (0.963) | (0.976) | (0.98) | (0.941) | (0.992) | (0.971) | (0.978) | (0.983) |
| | BLEED | 18/109 | 42/254 | 53/205 | 23/101 | 67/252 | 56/208 | 95/459 | 123/460 |
| | | (0.165) | (0.165) | (0.259) | (0.228) | (0.266) | (0.269) | (0.207) | (0.267) |
| | THROMBOTIC | 8/109 | 16/254 | 15/205 | 11/101 | 18/252 | 11/208 | 31/459 | 29/460 |
| | | (0.073) | (0.063) | (0.073) | (0.109) | (0.071) | (0.053) | (0.068) | (0.063) |
| SAE | ANY | 10/109 | 27/254 | 28/205 | 14/101 | 26/252 | 25/208 | 55/459 | 51/460 |
| | | (0.092) | (0.106) | (0.137) | (0.139) | (0.103) | (0.12) | (0.12) | (0.111) |
| | BLEED | 3/109 | 2/254 | 6/205 | 1/101 | 10/252 | 10/208 | 8/459 | 20/460 |
| | | (0.028) | (0.008) | (0.029) | (0.01) | (0.04) | (0.048) | (0.017) | (0.043) |
| | THROMBOTIC | 2/109 | 9/254 | 8/205 | 4/101 | 4/252 | 4/208 | 17/459 | 8/460 |
| | | (0.018) | (0.035) | (0.039) | (0.04) | (0.016) | (0.019) | (0.037) | (0.017) |

TABLE 65 shows logistic regression statistics comparing frequency of adverse and serious adverse events with response to XIGRIS™ by SERPINE1 rs7242 genotypes in all PROWESS placebo- and XIGRIS™-treated subjects with two or more organ dysfunctions (MOD ≧2). Within the placebo group the TT group showed a trend for more adverse bleeding events relative to the GG group (p=0.062). With XIGRIS™-treatment the GT group showed a trend for more serious adverse bleeding events relative to the GG group (p=0.055).

TABLE 65
Logistic Regression of adverse and serious adverse events by
genotype + treatment (XIGRIS ™) of SERPINE1 rs7242
in both a recessive and categorical model for all subjects with
two or more organ dysfunctions (MOD ≧ 2) in the PROWESS
Severe Sepsis Cohort (base: categorical and recessive model = GG).

| | | | | Estimate | | |
|---|---|---|---|---|---|---|
| | | | | of Log | Std. | |
| | adverse event | model | term | Odds | Error | P value |
| AE | ANY | categorical | GT | 0.454 | 0.656 | 0.489 |
| | ANY | categorical | TT | 0.649 | 0.717 | 0.365 |
| | ANY | categorical | treatment | −0.506 | 0.661 | 0.444 |
| | ANY | categorical | GT*treatment | 1.612 | 1.054 | 0.126 |
| | ANY | categorical | TT*treatment | 0.105 | 0.929 | 0.91 |
| | ANY | recessive | TT/GT | 0.537 | 0.601 | 0.372 |
| | ANY | recessive | treatment | −0.506 | 0.661 | 0.444 |
| | ANY | recessive | TT/GT*treatment | 0.735 | 0.816 | 0.368 |
| | BLEED | categorical | GT | 0.002 | 0.308 | 0.996 |
| | BLEED | categorical | TT | 0.567 | 0.303 | 0.062 |
| | BLEED | categorical | treatment | 0.399 | 0.35 | 0.255 |
| | BLEED | categorical | GT*treatment | 0.204 | 0.414 | 0.623 |
| | BLEED | categorical | TT*treatment | −0.344 | 0.416 | 0.408 |
| | BLEED | recessive | TT/GT | 0.277 | 0.283 | 0.326 |
| | BLEED | recessive | treatment | 0.399 | 0.35 | 0.255 |
| | BLEED | recessive | TT/GT*treatment | −0.064 | 0.384 | 0.868 |
| | THROMBOTIC | categorical | GT | −0.164 | 0.449 | 0.715 |
| | THROMBOTIC | categorical | TT | −0.003 | 0.455 | 0.994 |
| | THROMBOTIC | categorical | treatment | 0.434 | 0.487 | 0.373 |
| | THROMBOTIC | categorical | GT*treatment | −0.299 | 0.603 | 0.62 |
| | THROMBOTIC | categorical | TT*treatment | −0.78 | 0.636 | 0.22 |
| | THROMBOTIC | recessive | TT/GT | −0.089 | 0.412 | 0.828 |
| | THROMBOTIC | recessive | treatment | 0.434 | 0.487 | 0.373 |
| | THROMBOTIC | recessive | TT/GT*treatment | −0.507 | 0.555 | 0.361 |
| SAE | ANY | categorical | GT | 0.163 | 0.389 | 0.675 |
| | ANY | categorical | TT | 0.449 | 0.389 | 0.249 |
| | ANY | categorical | treatment | 0.466 | 0.439 | 0.289 |
| | ANY | categorical | GT*treatment | −0.499 | 0.527 | 0.343 |
| | ANY | categorical | TT*treatment | −0.612 | 0.529 | 0.247 |
| | ANY | recessive | TT/GT | 0.298 | 0.362 | 0.409 |
| | ANY | recessive | treatment | 0.466 | 0.439 | 0.289 |
| | ANY | recessive | TT/GT*treatment | −0.553 | 0.486 | 0.254 |
| | BLEED | categorical | GT | −1.271 | 0.92 | 0.167 |
| | BLEED | categorical | TT | 0.063 | 0.717 | 0.93 |
| | BLEED | categorical | treatment | −1.04 | 1.163 | 0.371 |
| | BLEED | categorical | GT*treatment | 2.69 | 1.4 | 0.055 |
| | BLEED | categorical | TT*treatment | 1.556 | 1.277 | 0.223 |
| | BLEED | recessive | TT/GT | −0.467 | 0.686 | 0.496 |
| | BLEED | recessive | treatment | −1.04 | 1.163 | 0.371 |
| | BLEED | recessive | TT/GT*treatment | 1.981 | 1.238 | 0.109 |
| | THROMBOTIC | categorical | GT | 0.676 | 0.79 | 0.393 |
| | THROMBOTIC | categorical | TT | 0.776 | 0.8 | 0.332 |
| | THROMBOTIC | categorical | treatment | 0.791 | 0.877 | 0.367 |
| | THROMBOTIC | categorical | GT*treatment | −1.614 | 1.067 | 0.13 |
| | THROMBOTIC | categorical | TT*treatment | −1.519 | 1.075 | 0.157 |
| | THROMBOTIC | recessive | TT/GT | 0.722 | 0.755 | 0.339 |
| | THROMBOTIC | recessive | treatment | 0.791 | 0.877 | 0.367 |
| | THROMBOTIC | recessive | TT/GT*treatment | −1.567 | 0.979 | 0.109 |

Note.
treatment = treatment with XIGRIS ™

TABLE 66 compares adverse and serious advents events in PROWESS placebo vs. XIGRIS™-treated subjects with two or more organ dysfunctions (MOD ≧2) by SERPINE1 rs7242 genotype using an exact test approach. The GT group had significantly more adverse bleeding events in the XIGRIS™-treated vs. placebo subjects (p=0.007). The TT/GT group had significantly more adverse bleeding events in the XIGRIS™-treated vs. placebo subjects (p=0.036). The GT group showed significantly more serious adverse bleeding events in the XIGRIS™-treated vs. placebo subjects (p=0.021). The TT/GT group had significantly more serious adverse bleeding events in the XIGRIS™-treated vs. placebo subjects (p=0.033).

TABLE 66
An Exact test of adverse and serious adverse events by genotype
against treatment (XIGRIS ™) of SERPINE1 rs7242 for all
subjects with two or more organ dysfunctions (MOD ≧ 2) in the
PROWESS Severe Sepsis Cohort.

| | variable | group | OddsRatio | P.Value |
|---|---|---|---|---|
| AE | ANY | GG | 0.605 | 0.526 |
| | ANY | GT | 3.018 | 0.285 |
| | ANY | TT | 0.671 | 0.751 |
| | ANY | TT/GT | 1.258 | 0.644 |
| | BLEED | GG | 1.488 | 0.297 |
| | BLEED | GT | 1.826 | 0.007 |
| | BLEED | TT | 1.056 | 0.824 |
| | BLEED | TT/GT | 1.398 | 0.036 |
| | THROMBOTIC | GG | 1.54 | 0.472 |
| | THROMBOTIC | GT | 1.144 | 0.726 |
| | THROMBOTIC | TT | 0.708 | 0.424 |
| | THROMBOTIC | TT/GT | 0.929 | 0.791 |
| SAE | ANY | GG | 1.59 | 0.386 |
| | ANY | GT | 0.967 | 1 |
| | ANY | TT | 0.864 | 0.661 |
| | ANY | TT/GT | 0.916 | 0.681 |
| | BLEED | GG | 0.355 | 0.622 |
| | BLEED | GT | 5.192 | 0.021 |
| | BLEED | TT | 1.673 | 0.446 |
| | BLEED | TT/GT | 2.56 | 0.033 |
| | THROMBOTIC | GG | 2.198 | 0.431 |
| | THROMBOTIC | GT | 0.44 | 0.261 |
| | THROMBOTIC | TT | 0.484 | 0.257 |
| | THROMBOTIC | TT/GT | 0.461 | 0.071 |

4.3.1 Incidence of Adverse Outcomes and Response to XIGRIS™ by SERPINE1 rs7242 and PROC rs2069912 Genotype Combination for all Subjects in the PROWESS Severe Sepsis Cohort

TABLE 67 shows the adverse and serious adverse events for all PROWESS placebo- and XIGRIS™-treated subjects by SERPINE1 rs7242 and PROC rs2069912 genotype combination. An increase in adverse thrombotic events is observed in the NRGC XIGRIS™-treated group (17.8%) vs. the NRGC placebo group (9.1%). In contrast a decrease in adverse thrombotic events is observed in the IRGC XIGRIS™-treated group (5.2%) vs. the IRGC placebo group (7.2%). An increase in serious adverse events is observed in the NRGC XIGRIS™-treated (19.2%) vs. the NRGC placebo group (5.2%). In contrast there was a decrease in serious adverse events observed in the IRGC XIGRIS™-treated group (10.5%) vs. the IRGC placebo group (13.8%). An increase in serious adverse thrombotic events is observed in the NRGC XIGRIS™-treated group (5.5%) vs. the NRGC placebo group (1.3%). In contrast a decrease in serious adverse thrombotic events is observed in the IRGC XIGRIS™-treated group (1.6%) vs. the IRGC placebo group (3.3%).

TABLE 67
Incidence of adverse and serious adverse events by genotype
of SERPINE1 rs7242 and PROC rs2069912 genotype combination
in placebo and treated (XIGRIS ™) subjects in the
PROWESS Severe Sepsis Cohort (data is presented
as # of events/individuals (fraction)).

| | | Placebo | | | Xigris | | |
|---|---|---|---|---|---|---|---|
| | | NRGC | MRGC | IRGC | NRGC | MRGC | IRGC |
| AE | ANY | 74/77 | 384/392 | 270/276 | 70/73 | 402/416 | 256/258 |
| | | (0.961) | (0.98) | (0.978) | (0.959) | (0.966) | (0.992) |
| | BLEED | 11/77 | 75/392 | 60/276 | 17/73 | 105/416 | 67/258 |
| | | (0.143) | (0.191) | (0.217) | (0.233) | (0.252) | (0.26) |
| | THROMBOTIC | 7/77 | 26/392 | 20/276 | 13/73 | 28/416 | 13/258 |
| | | (0.091) | (0.066) | (0.072) | (0.178) | (0.067) | (0.05) |
| SAE | ANY | 4/77 | 43/392 | 38/276 | 14/73 | 51/416 | 27/258 |
| | | (0.052) | (0.11) | (0.138) | (0.192) | (0.123) | (0.105) |
| | BLEED | 0/77 (0) | 7/392 | 4/276 | 3/73 | 12/416 | 10/258 |
| | | | (0.018) | (0.014) | (0.041) | (0.029) | (0.039) |
| | THROMBOTIC | 1/77 | 13/392 | 9/276 | 4/73 | 8/416 | 4/258 |
| | | (0.013) | (0.033) | (0.033) | (0.055) | (0.019) | (0.016) |

TABLE 68 shows logistic regression statistics comparing frequency of adverse and serious adverse events with response to XIGRIS™ by SERPINE1 rs7242 and PROC rs2069912 genotype combination in all PROWESS placebo- and XIGRIS™-treated subjects. With XIGRIS™ treatment the IRGC group showed a trend for less adverse thrombotic events relative to the NRGC group (p=0.062). Within the placebo group the IRGC group showed significantly more serious adverse events relative to the NRGC group (p=0.049). With XIGRIS™ treatment the MRGC group showed significantly less serious adverse events relative to the NRGC group (p=0.034). With XIGRIS™ treatment the IRGC group showed a significantly less serious adverse events relative to the NRGC group (p=0.006). With XIGRIS™ treatment the MRGC group showed a trend for less serious adverse thrombotic events relative to the NRGC group (p=0.094). With XIGRIS™ treatment the IRGC group showed a trend for less serious adverse thrombotic events relative to the NRGC group (p=0.08).

TABLE 68
Logistic Regression of adverse and serious adverse events by
genotype + treatment (XIGRIS ™) of SERPINE1 rs7242
and PROC rs2069912 genotype combination in both a
recessive and categorical model for all subjects in the PROWESS
Severe Sepsis Cohort (base = NRGC genotype group).

| | | | Estimate | | |
|---|---|---|---|---|---|
| | | | of Log | Std. | P |
| | adverse event | term | Odds | Error | value |
| AE | ANY | MRGC | 0.666 | 0.689 | 0.334 |
| | ANY | IRGC | 0.601 | 0.719 | 0.403 |
| | ANY | treatment | −0.056 | 0.833 | 0.947 |
| | ANY | MRGC*treatment | −0.458 | 0.947 | 0.628 |
| | ANY | IRGC*treatment | 1.101 | 1.17 | 0.347 |
| | BLEED | MRGC | 0.35 | 0.35 | 0.317 |
| | BLEED | IRGC | 0.511 | 0.357 | 0.152 |
| | BLEED | treatment | 0.6 | 0.427 | 0.161 |
| | BLEED | MRGC*treatment | −0.244 | 0.46 | 0.596 |
| | BLEED | IRGC*treatment | −0.366 | 0.473 | 0.439 |
| | THROMBOTIC | MRGC | −0.342 | 0.445 | 0.443 |
| | THROMBOTIC | IRGC | −0.247 | 0.459 | 0.591 |
| | THROMBOTIC | treatment | 0.773 | 0.501 | 0.123 |
| | THROMBOTIC | MRGC*treatment | −0.757 | 0.575 | 0.187 |
| | THROMBOTIC | IRGC*treatment | −1.16 | 0.621 | 0.062 |
| SAE | ANY | MRGC | 0.81 | 0.538 | 0.132 |
| | ANY | IRGC | 1.069 | 0.542 | 0.049 |
| | ANY | treatment | 1.466 | 0.593 | 0.013 |
| | ANY | MRGC*treatment | −1.34 | 0.633 | 0.034 |
| | ANY | IRGC*treatment | −1.778 | 0.651 | 0.006 |
| | BLEED* | MRGC | 15.046 | 948.516 | 0.987 |
| | BLEED* | IRGC | 14.834 | 948.517 | 0.988 |
| | BLEED* | treatment | 15.904 | 948.517 | 0.987 |
| | BLEED* | MRGC*treatment | −15.413 | 948.517 | 0.987 |
| | BLEED* | IRGC*treatment | −14.895 | 948.517 | 0.987 |
| | THROMBOTIC | MRGC | 0.958 | 1.045 | 0.359 |
| | THROMBOTIC | IRGC | 0.941 | 1.062 | 0.376 |
| | THROMBOTIC | treatment | 1.483 | 1.13 | 0.19 |
| | THROMBOTIC | MRGC*treatment | −2.042 | 1.218 | 0.094 |
| | THROMBOTIC | IRGC*treatment | −2.244 | 1.283 | 0.08 |

*If one arm of SAE event is too small then the power is too small and the logistic regression results become un-reliable.
Note.
treatment = treatment with XIGRIS ™

For fixed sample size the smaller power we have. So when we have too small number of a certain SAE events, logistic regression is not a good tool to test the difference.

TABLE 69 compares adverse and serious advents events in PROWESS placebo vs. XIGRIS™-treated subjects by SERPINE1 rs7242 and PROC rs2069912 genotype combination using an exact test approach. The MRGC group had significantly more adverse bleeding events in the XIGRIS™-treated vs. placebo subjects (p=0.042). The NRGC group had significantly more serious adverse events in the XIGRIS™-treated vs. placebo subjects (p=0.011).

TABLE 69
An Exact test of adverse and serious adverse events by genotype
against treatment (XIGRIS ™) of SERPINE1 rs7242 and
PROC rs2069912 genotype combination for all subjects in
the PROWESS Severe Sepsis Cohort.

| | variable | group | OddsRatio | P.Value |
|---|---|---|---|---|
| AE | ANY | NRGC | 0.946 | 1 |
| | ANY | MRGC | 0.599 | 0.284 |
| | ANY | IRGC | 2.84 | 0.288 |
| | BLEED | NRGC | 1.814 | 0.209 |
| | BLEED | MRGC | 1.426 | 0.042 |
| | BLEED | IRGC | 1.262 | 0.264 |
| | THROMBOTIC | NRGC | 2.156 | 0.151 |
| | THROMBOTIC | MRGC | 1.016 | 1 |
| | THROMBOTIC | IRGC | 0.68 | 0.369 |
| SAE | ANY | NRGC | 4.291 | 0.011 |
| | ANY | MRGC | 1.134 | 0.585 |
| | ANY | IRGC | 0.732 | 0.29 |
| | BLEED | NRGC | Inf | 0.113 |
| | BLEED | MRGC | 1.633 | 0.358 |
| | BLEED | IRGC | 2.737 | 0.104 |
| | THROMBOTIC | NRGC | 4.368 | 0.2 |
| | THROMBOTIC | MRGC | 0.572 | 0.27 |
| | THROMBOTIC | IRGC | 0.468 | 0.265 |

4.3.2 Incidence of Adverse Outcomes and Response to XIGRIS™ by SERPINE1 rs7242 and PROC rs2069912 Genotype Combination for all Subjects with APACHE II ≧25 in the PROWESS Severe Sepsis Cohort

TABLE 70 shows the adverse and serious adverse events for all PROWESS placebo- and XIGRIS™-treated subjects with an APACHE II ≧25 by SERPINE1 rs7242 and PROC rs2069912 genotype combination. An increase in adverse thrombotic events is observed in the NRGC XIGRIS™-treated group (27.3%) vs. the NRGC placebo group (7.9%). In contrast a decrease in adverse thrombotic events is observed in the IRGC XIGRIS™-treated group (7.3%) vs. the IRGC placebo group (8.7%). An increase in serious adverse events is observed in the NRGC XIGRIS™-treated (21.2%) vs. the NRGC placebo group (2.6%). In contrast there was a decrease in serious adverse events observed in the IRGC XIGRIS™-treated group (11.3%) vs. the IRGC placebo group (18.1%). An increase in serious adverse thrombotic events is observed in the NRGC XIGRIS™-treated group (9.1%) vs. the NRGC placebo group (0%). In contrast a decrease in serious adverse thrombotic events is observed in the IRGC XIGRIS™-treated group (1.6%) vs. the IRGC placebo group (3.6%).

TABLE 70
Incidence of adverse and serious adverse events by genotype
of SERPINE1 rs7242 and PROC rs2069912 genotype combination
in placebo and treated (XIGRIS ™) subjects with
APACHE II ≧ 25 in the PROWESS Severe Sepsis Cohort
(data is presented as # of events/individuals (fraction)).

| | | Placebo | | | Xigris | | |
|---|---|---|---|---|---|---|---|
| | | NRGC | MRGC | IRGC | NRGC | MRGC | IRGC |
| AE | ANY | 37/38 | 173/178 | 137/138 | 32/33 | 200/204 | 123/124 |
| | | (0.974) | (0.972) | (0.993) | (0.97) | (0.98) | (0.992) |
| | BLEED | 5/38 | 40/178 | 33/138 | 12/33 | 57/204 | 41/124 |
| | | (0.132) | (0.225) | (0.239) | (0.364) | (0.279) | (0.331) |
| | THROMBOTIC | 3/38 | 15/178 | 12/138 | 9/33 | 17/204 | 9/124 |
| | | (0.079) | (0.084) | (0.087) | (0.273) | (0.083) | (0.073) |
| SAE | ANY | 1/38 | 23/178 | 25/138 | 7/33 | 29/204 | 14/124 |
| | | (0.026) | (0.129) | (0.181) | (0.212) | (0.142) | (0.113) |
| | BLEED | 0/38 (0) | 3/178 | 1/138 | 1/33 | 6/204 | 7/124 |
| | | | (0.017) | (0.007) | (0.03) | (0.029) | (0.056) |
| | THROMBOTIC | 0/38 (0) | 8/178 | 7/138 | 3/33 | 7/204 | 3/124 |
| | | | (0.045) | (0.051) | (0.091) | (0.034) | (0.024) |

TABLE 71 shows logistic regression statistics comparing frequency of adverse and serious adverse events with response to XIGRIS™ by SERPINE1 rs7242 and PROC rs2069912 genotype combination in all PROWESS placebo- and XIGRIS™-treated subjects with an APACHE II ≧25. With XIGRIS™ treatment the MRGC group showed a trend for less adverse thrombotic events relative to the NRGC group (p=0.065). With XIGRIS™ treatment the IRGC group showed significantly less adverse thrombotic events relative to the NRGC group (p=0.05). Within the placebo group the IRGC group showed significantly more serious adverse events relative to the NRGC group (p=0.043). With XIGRIS™ treatment the MRGC group showed a trend for less serious adverse events relative to the NRGC group (p=0.055). With XIGRIS™ treatment the IRGC group showed significantly less serious adverse events relative to the NRGC group (p=0.014).

TABLE 71
Logistic Regression of adverse and serious adverse events by
genotype + treatment (XIGRIS ™) of SERPINE1 rs7242
and PROC rs2069912 genotype combination in both a
recessive and categorical model for all subjects with
APACHE II ≧ 25 in the PROWESS Severe
Sepsis Cohort (base = NRGC genotype group).

| | | | Estimate | | |
|---|---|---|---|---|---|
| | | | of Log | Std. | P |
| | adverse event | term | Odds | Error | value |
| AE | ANY | MRGC | −0.067 | 1.11 | 0.952 |
| | ANY | IRGC | 1.309 | 1.426 | 0.359 |
| | ANY | treatment | −0.145 | 1.435 | 0.919 |
| | ANY | MRGC*treatment | 0.513 | 1.587 | 0.746 |
| | ANY | IRGC*treatment | 0.037 | 2.018 | 0.985 |
| | BLEED | MRGC | 0.649 | 0.512 | 0.206 |
| | BLEED | IRGC | 0.73 | 0.52 | 0.16 |
| | BLEED | treatment | 1.327 | 0.601 | 0.027 |
| | BLEED | MRGC*treatment | −1.036 | 0.646 | 0.109 |
| | BLEED | IRGC*treatment | −0.875 | 0.661 | 0.186 |
| | THROMBOTIC | MRGC | 0.071 | 0.659 | 0.914 |
| | THROMBOTIC | IRGC | 0.105 | 0.673 | 0.876 |
| | THROMBOTIC | treatment | 1.476 | 0.717 | 0.04 |
| | THROMBOTIC | MRGC*treatment | −1.488 | 0.807 | 0.065 |
| | THROMBOTIC | IRGC*treatment | −1.672 | 0.852 | 0.05 |
| SAE | ANY | MRGC | 1.703 | 1.038 | 0.101 |
| | ANY | IRGC | 2.102 | 1.037 | 0.043 |
| | ANY | treatment | 2.299 | 1.099 | 0.037 |
| | ANY | MRGC*treatment | −2.188 | 1.14 | 0.055 |
| | ANY | IRGC*treatment | −2.852 | 1.157 | 0.014 |
| | BLEED* | MRGC | 14.298 | 956.543 | 0.988 |
| | BLEED* | IRGC | 13.444 | 956.544 | 0.989 |
| | BLEED* | treatment | 14.899 | 956.544 | 0.988 |
| | BLEED* | MRGC*treatment | −14.329 | 956.544 | 0.988 |
| | BLEED* | IRGC*treatment | −12.795 | 956.544 | 0.989 |
| | THROMBOTIC* | MRGC | 15.168 | 956.543 | 0.987 |
| | THROMBOTIC* | IRGC | 15.083 | 956.543 | 0.987 |
| | THROMBOTIC* | treatment | 16.062 | 956.543 | 0.987 |
| | THROMBOTIC* | MRGC*treatment | −16.362 | 956.544 | 0.986 |
| | THROMBOTIC* | IRGC*treatment | −16.892 | 956.544 | 0.986 |

*If one arm of SAE event is too small then the power is too small and the logistic regression results become un-reliable.
Note.
treatment = treatment with XIGRIS ™

TABLE 72 compares adverse and serious advents events in PROWESS placebo vs. XIGRIS™-treated subjects with an APACHE II ≧25 by SERPINE1 rs7242 and PROC rs2069912 genotype combination using an exact test approach. The NRGC group had significantly more adverse bleeding events in the XIGRIS™-treated vs. placebo subjects (p=0.028). The NRGC group had a trend for more adverse thrombotic events in the XIGRIS™-treated vs. placebo subjects (p=0.054). The NRGC group had significantly more serious adverse events in the XIGRIS™-treated vs. placebo subjects (p=0.021). The IRGC group had significantly more serious adverse bleeding events in the XIGRIS™-treated vs. placebo subjects (p=0.029). The NRGC group had a trend for more serious adverse thrombotic events in the XIGRIS™-treated vs. placebo subjects (p=0.095).

TABLE 72
An Exact test of adverse and serious adverse events by genotype
against treatment (XIGRIS ™) of SERPINE1 rs7242 and
PROC rs2069912 genotype combination for all subjects
with APACHE II ≧ 25 in the PROWESS Severe
Sepsis Cohort.

| | variable | group | OddsRatio | P.Value |
|---|---|---|---|---|
| AE | ANY | NRGC | 0.867 | 1 |
| | ANY | MRGC | 1.444 | 0.739 |
| | ANY | IRGC | 0.898 | 1 |
| | BLEED | NRGC | 3.699 | 0.028 |
| | BLEED | MRGC | 1.337 | 0.24 |
| | BLEED | IRGC | 1.569 | 0.13 |
| | THROMBOTIC | NRGC | 4.285 | 0.054 |
| | THROMBOTIC | MRGC | 0.988 | 1 |
| | THROMBOTIC | IRGC | 0.822 | 0.82 |
| SAE | ANY | NRGC | 9.681 | 0.021 |
| | ANY | MRGC | 1.116 | 0.766 |
| | ANY | IRGC | 0.576 | 0.164 |
| | BLEED | NRGC | Inf | 0.465 |
| | BLEED | MRGC | 1.765 | 0.512 |
| | BLEED | IRGC | 8.144 | 0.029 |
| | THROMBOTIC | NRGC | Inf | 0.095 |
| | THROMBOTIC | MRGC | 0.741 | 0.59 |
| | THROMBOTIC | IRGC | 0.437 | 0.34 |

4.3.3 Incidence of Adverse Outcomes and Response to XIGRIS™ by SERPINE1 rs7242 and PROC rs2069912 Genotype Combination for all Subjects with Two or More Organ Dysfunctions (MOD ≧2) in the PROWESS Severe Sepsis Cohort

TABLE 73 shows the adverse and serious adverse events for all PROWESS placebo- and XIGRIS™-treated subjects with two or more organ dysfunctions (MOD ≧2) by SERPINE1 rs7242 and PROC rs2069912 genotype combination. An increase in adverse thrombotic events is observed in the NRGC XIGRIS™-treated group (17.3%) vs. the NRGC placebo group (6.8%). In contrast a decrease in adverse thrombotic events is observed in the IRGC XIGRIS™-treated group (4.7%) vs. the IRGC placebo group (6.9%). An increase in serious adverse events is observed in the NRGC XIGRIS™-treated (21.2%) vs. the NRGC placebo group (3.4%). In contrast there was a decrease in serious adverse events observed in the IRGC XIGRIS™-treated group (9.9%) vs. the IRGC placebo group (11.8%). An increase in serious adverse thrombotic events is observed in the NRGC XIGRIS™-treated (5.8%) vs. the NRGC placebo group (0%). In contrast there was a decrease in serious adverse thrombotic events observed in the IRGC XIGRIS™-treated group (1%) vs. the IRGC placebo group (2.9%).

TABLE 73
Incidence of adverse and serious adverse events by genotype
of SERPINE1 rs7242 and PROC rs2069912 genotype combination
in placebo and treated (XIGRIS ™) subjects with
two or more organ dysfunctions (MOD ≧ 2) in the PROWESS
Severe Sepsis Cohort (data is presented as # of
events/individuals (fraction)).

| | | Placebo | | | Xigris | | |
|---|---|---|---|---|---|---|---|
| | | NRGC | MRGC | IRGC | NRGC | MRGC | IRGC |
| AE | ANY | 56/59 | 293/299 | 199/204 | 49/52 | 305/314 | 190/192 |
| | | (0.949) | (0.98) | (0.975) | (0.942) | (0.971) | (0.99) |
| | BLEED | 7/59 | 59/299 | 44/204 | 12/52 | 77/314 | 56/192 |
| | | (0.119) | (0.197) | (0.216) | (0.231) | (0.245) | (0.292) |
| | THROMBOTIC | 4/59 | 21/299 | 14/204 | 9/52 | 22/314 | 9/192 |
| | | (0.068) | (0.07) | (0.069) | (0.173) | (0.07) | (0.047) |
| SAE | ANY | 2/59 | 38/299 | 24/204 | 11/52 | 34/314 | 19/192 |
| | | (0.034) | (0.127) | (0.118) | (0.212) | (0.108) | (0.099) |
| | BLEED | 0/59 (0) | 7/299 | 3/204 | 1/52 | 9/314 | 10/192 |
| | | | (0.023) | (0.015) | (0.019) | (0.029) | (0.052) |
| | THROMBOTIC | 0/59 (0) | 13/299 | 6/204 | 3/52 | 7/314 | 2/192 |
| | | | (0.043) | (0.029) | (0.058) | (0.022) | (0.01) |

TABLE 74 shows logistic regression statistics comparing frequency of adverse and serious adverse events with response to XIGRIS™ by SERPINE1 rs7242 and PROC rs2069912 genotype combination in all PROWESS placebo- and XIGRIS™-treated subjects with two or more organ dysfunctions (MOD ≧2). Treatment with XIGRIS™ shows a trend for more adverse thrombotic events relative to no treatment (p=0.096). With XIGRIS™ treatment the IRGC group shows a trend for less adverse thrombotic events relative to the NRGC group (p=0.058). Within the placebo group the MRGC group showed a trend for more serious adverse events relative to the NRGC group (p=0.054). Within the placebo group the IRGC group showed a trend for more serious adverse events relative to the NRGC group (p=0.076). With XIGRIS™ treatment the MRGC group showed significantly less serious adverse events relative to the NRGC group (p=0.008). With XIGRIS™ treatment the IRGC group showed significantly less serious adverse events relative to the NRGC group (p=0.01).

TABLE 74
Logistic Regression of adverse and serious adverse events by
genotype + treatment (XIGRIS ™) of SERPINE1 rs7242
and PROC rs2069912 genotype combination in both a
recessive and categorical model for all subjects with two or more
organ dysfunctions (MOD ≧ 2) in the PROWESS Severe Sepsis
Cohort (base = NRGC genotype group).

| | | | Estimate | | |
|---|---|---|---|---|---|
| | | | of Log | Std. | P |
| | adverse event | term | Odds | Error | value |
| AE | ANY | MRGC | 0.962 | 0.722 | 0.183 |
| | ANY | IRGC | 0.757 | 0.746 | 0.31 |
| | ANY | treatment | −0.134 | 0.84 | 0.874 |
| | ANY | MRGC*treatment | −0.232 | 0.995 | 0.816 |
| | ANY | IRGC*treatment | 1.004 | 1.19 | 0.399 |
| | BLEED | MRGC | 0.602 | 0.428 | 0.159 |
| | BLEED | IRGC | 0.714 | 0.437 | 0.102 |
| | BLEED | treatment | 0.801 | 0.52 | 0.123 |
| | BLEED | MRGC*treatment | −0.523 | 0.556 | 0.347 |
| | BLEED | IRGC*treatment | −0.398 | 0.57 | 0.485 |
| | THROMBOTIC | MRGC | 0.038 | 0.565 | 0.946 |
| | THROMBOTIC | IRGC | 0.013 | 0.587 | 0.982 |
| | THROMBOTIC | treatment | 1.057 | 0.634 | 0.096 |
| | THROMBOTIC | MRGC*treatment | −1.06 | 0.709 | 0.135 |
| | THROMBOTIC | IRGC*treatment | −1.461 | 0.772 | 0.058 |
| SAE | ANY | MRGC | 1.423 | 0.74 | 0.054 |
| | ANY | IRGC | 1.335 | 0.751 | 0.076 |
| | ANY | treatment | 2.034 | 0.795 | 0.011 |
| | ANY | MRGC*treatment | −2.216 | 0.834 | 0.008 |
| | ANY | IRGC*treatment | −2.228 | 0.859 | 0.01 |
| | BLEED* | MRGC | 15.062 | 950.907 | 0.987 |
| | BLEED* | IRGC | 14.588 | 950.907 | 0.988 |
| | BLEED* | treatment | 14.861 | 950.908 | 0.988 |
| | BLEED* | MRGC*treatment | −14.653 | 950.908 | 0.988 |
| | BLEED* | IRGC*treatment | −13.557 | 950.908 | 0.989 |
| | THROMBOTIC* | MRGC | 15.701 | 950.907 | 0.987 |
| | THROMBOTIC* | IRGC | 15.296 | 950.907 | 0.987 |
| | THROMBOTIC* | treatment | 15.999 | 950.907 | 0.987 |
| | THROMBOTIC* | MRGC*treatment | −16.689 | 950.907 | 0.986 |
| | THROMBOTIC* | IRGC*treatment | −17.057 | 950.908 | 0.986 |

*If one arm of SAE event is too small then the power is too small and the logistic regression results become un-reliable.
Note.
treatment = treatment with XIGRIS ™

TABLE 75 compares adverse and serious advents events in PROWESS placebo vs. XIGRIS™-treated subjects with two or more organ dysfunctions (MOD ≧2) by SERPINE1 rs7242 and PROC rs2069912 genotype combination using an exact test approach. The IRGC group had a trend for more adverse bleeding events in the XIGRIS™-treated vs. placebo subjects (p=0.084). The NRGC group had significantly more serious adverse events in the XIGRIS™-treated vs. placebo subjects (p=0.006). The IRGC group had significantly more serious adverse bleeding events in the XIGRIS™-treated vs. placebo subjects (p=0.048).

TABLE 75
An Exact test of adverse and serious adverse events by genotype
against treatment (XIGRIS ™) of SERPINE1 rs7242 and
PROC rs2069912 genotype combination for all subjects
with two or more organ dysfunctions (MOD ≧ 2) in the
PROWESS Severe Sepsis Cohort.

| | variable | group | OddsRatio | P.Value |
|---|---|---|---|---|
| AE | ANY | NRGC | 0.876 | 1 |
| | ANY | MRGC | 0.694 | 0.604 |
| | ANY | IRGC | 2.382 | 0.45 |
| | BLEED | NRGC | 2.212 | 0.136 |
| | BLEED | MRGC | 1.321 | 0.174 |
| | BLEED | IRGC | 1.496 | 0.084 |
| | THROMBOTIC | NRGC | 2.851 | 0.137 |
| | THROMBOTIC | MRGC | 0.997 | 1 |
| | THROMBOTIC | IRGC | 0.668 | 0.396 |
| SAE | ANY | NRGC | 7.521 | 0.006 |
| | ANY | MRGC | 0.834 | 0.531 |
| | ANY | IRGC | 0.824 | 0.629 |
| | BLEED | NRGC | Inf | 0.468 |
| | BLEED | MRGC | 1.23 | 0.802 |
| | BLEED | IRGC | 3.67 | 0.048 |
| | THROMBOTIC | NRGC | Inf | 0.1 |
| | THROMBOTIC | MRGC | 0.502 | 0.174 |
| | THROMBOTIC | IRGC | 0.348 | 0.286 |

1. A method of treating an inflammatory condition in a subject in need thereof, the method comprising administering to the subject an anti-inflammatory agent or an anti-coagulant agent, wherein said subject is determined to have an improved response genotype at one or more of the following sites: rs7242; rs2070682; rs11178; rs2227706; and rs2227684; or one or more polymorphic sites in linkage disequilibrium thereto.

2. (canceled)

3. The method of claim 1, further comprising determining a genotype of said subject at rs2069912 or one or more polymorphic sites in linkage disequilibrium thereto.

4.-6. (canceled)

7. The method of claim 1, further comprising determining the subject's APACHE II score as an assessment of subject risk or determining the number of organ system failures for the subject as an assessment of subject risk.

8. (canceled)

9. The method of claim 7, wherein the subject's APACHE II score is indicative of an increased risk when ≧25 or wherein 2 or more organ system failures are indicative of increased subject risk.

10. (canceled)

11. The method of claim 1, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung, kidney, bone marrow, graft-versus-host disease, transplant rejection, sickle cell anemia, nephrotic syndrome, toxicity of agents such as OKT3, cytokine therapy, and cirrhosis.

12. The method of claim 11, wherein the inflammatory condition is selected from: SIRS; sepsis; severe sepsis; and septic shock.

13. The method of claim 3, wherein the improved response genotype is selected from one or more of the following improved response genotypes, improved response genotype combinations or mixed response genotype combinations: rs7242 GT; rs7242 TT; rs2070682 CT; rs2070682 TT; rs11178 CT; rs11178 TT; rs2227706 AG; rs2227706 GG; rs2227684AG; rs2227684 GG; rs7242 GT/rs2069912 CC; rs7242 GT/rs2069912 CT; rs7242 GT/rs2069912 TT; rs7242 GG/rs2069912 CC; rs7242 TT/rs2069912 TT; rs7242 GG/rs2069912 CT; rs7242 TT/rs2069912 CT; rs7242 TT/rs2069912 CC; rs2070682 CT/rs2069912 CC; rs2070682 CT/rs2069912 CT; rs2070682 CT/rs2069912 TT; rs2070682 CC/rs2069912 CC; rs2070682 CC/rs2069912 CT; rs2070682 TT/rs2069912 TT; rs2070682 TT/rs2069912 CT; rs2070682 TT/rs2069912 CC; rs11178 CT/rs2069912 CC; rs11178 CT/rs2069912 CT; rs11178 CT/rs2069912 TT; rs11178 CC/rs2069912 CC; rs11178 CC/rs2069912 CT; rs11178 TT/rs2069912 TT; rs11178 TT/rs2069912 CT; rs11178 TT/rs2069912 CC; rs2227706 AG/rs2069912 CC; rs2227706 AG/rs2069912 CT; rs2227706 AG/rs2069912 TT; rs2227706 AA/rs2069912 CC; rs2227706 AA/rs2069912 CT; rs2227706 GG/rs2069912 TT; rs2227706 GG/rs2069912 CT; rs2227706 GG/rs2069912 CC; rs2227684 AG/rs2069912 CC; rs2227684 AG/rs2069912 CT; rs2227684 AG/rs2069912 TT; rs2227684 AA/rs2069912 CC; rs2227684 GG/rs2069912 TT; rs2227684 AA/rs2069912 CT; rs2227684 GG/rs2069912 CT; and rs2227684 GG/rs2069912 CC; or one or more polymorphic sites in linkage disequilibrium thereto selected from one or more of the polymorphic sites listed in TABLE 1B.

14. (canceled)

15. The method of claim 1, wherein the anti-inflammatory agent or the anti-coagulant agent is drotrecogin alfa (activated).

16. A method of treating SIRS, severe sepsis, sepsis, or septic shock in a subject in need thereof, the method comprising administering to the subject a protein C or protein C like compound, wherein said subject is determined to have an improved response genotype in one or more of the following sites: rs7242; rs2070682; rs11178; rs2227706; and rs2227684; or one or more polymorphic sites in linkage disequilibrium thereto.

17. (canceled)

18. The method of claim 16, further comprising determining a genotype of said subject at rs2069912 or one or more polymorphic sites in linkage disequilibrium thereto.

19.-20. (canceled)

21. The method of claim 16, wherein the protein C or protein C like compound is drotrecogin alfa (activated).

22. The method of claim 16, wherein the subject's improved response genotype is selected from the group consisting of: rs7242 GT/rs2069912 CC; rs7242 GT/rs2069912 CT; rs7242 TT/rs2069912 CT; and rs7242 TT/rs2069912 CC.

23. (canceled)

24. The method of claim 16, further comprising determining the subject's APACHE II score as an assessment of subject risk or wherein the subject's APACHE II score is indicative of an increased risk when ≧25.

25.-28. (canceled)

29. A method of selecting a subject for the treatment of an inflammatory condition with an anti-inflammatory agent or an anti-coagulant agent, comprising the step of identifying a subject having a reduced serious adverse event genotype in one or more of the following sites: rs2069912; rs7242; rs2070682; rs11178; rs2227706; and rs2227684; one or more polymorphic sites in linkage disequilibrium thereto; and combinations thereof, wherein the identification of a subject with the reduced serious adverse event genotype is predictive of increased responsiveness to the treatment of the inflammatory condition with the anti-inflammatory agent or the anti-coagulant agent.

30.-31. (canceled)

32. The method of claim 29, further comprising:
(a) determining the subject's APACHE II score, wherein a score of ≧25 is indicative of increased subject risk; or
(b) determining the number of the subject's organ system failures, wherein 2 or more organ system failures are indicative of increased subject risk.

33.-35. (canceled)

36. The method of claim 29, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung, kidney, bone marrow, graft-versus-host disease, transplant rejection, sickle cell anemia, nephrotic syndrome, toxicity of agents such as OKT3, cytokine therapy, and cirrhosis.

37. (canceled)

38. The method of claim 29, wherein the reduced serious adverse event genotype(s) is selected from one or more of the following improved response genotypes, improved response genotype combinations or mixed response genotype combinations: rs7242 GT; rs7242 TT; rs2070682 CT; rs2070682 TT; rs11178 CT; rs11178 TT; rs2227706 AG; rs2227706 GG; rs2227684AG; rs2227684 GG; rs7242 GT/rs2069912 CC; rs7242 GT/rs2069912 CT; rs7242 GT/rs2069912 TT; rs7242 GG/rs2069912 CC; rs7242 TT/rs2069912 TT; rs7242 GG/rs2069912 CT; rs7242 TT/rs2069912 CT; rs7242 TT/rs2069912 CC; rs2070682 CT/rs2069912 CC; rs2070682 CT/rs2069912 CT; rs2070682 CT/rs2069912 TT; rs2070682 CC/rs2069912 CC; rs2070682 CC/rs2069912 CT; rs2070682 TT/rs2069912 TT; rs2070682 TT/rs2069912 CT; rs2070682 TT/rs2069912 CC; rs11178 CT/rs2069912 CC; rs11178 CT/rs2069912 CT; rs11178 CT/rs2069912 TT; rs11178 CC/rs2069912 CC; rs11178 CC/rs2069912 CT; rs11178 TT/rs2069912 TT; rs11178 TT/rs2069912 CT; rs11178 TT/rs2069912 CC; rs2227706 AG/rs2069912 CC; rs2227706 AG/rs2069912 CT; rs2227706 AG/rs2069912 TT; rs2227706 AA/rs2069912 CC; rs2227706 AA/rs2069912 CT; rs2227706 GG/rs2069912 TT; rs2227706 GG/rs2069912 CT; rs2227706 GG/rs2069912 CC; rs2227684 AG/rs2069912 CC; rs2227684 AG/rs2069912 CT; rs2227684 AG/rs2069912 TT; rs2227684 AA/rs2069912 CC; rs2227684 GG/rs2069912 TT; rs2227684 AA/rs2069912 CT; rs2227684 GG/rs2069912 CT; and rs2227684 GG/rs2069912 CC; or one or more polymorphic sites in linkage disequilibrium thereto selected from one or more of the polymorphic sites listed in TABLE 1B.

39. (canceled)

40. The method of claim 29, wherein the anti-inflammatory agent or the anti-coagulant agent is drotrecogin alfa (activated).

41. A method for identifying a subject having one or more serious adverse event genotype(s), the method comprising determining a genotype of said subject at one or more polymorphic sites, wherein said genotype is indicative of the subject's increased likelihood of having a serious adverse event in response to the administration of an anti-inflammatory agent or an anti-coagulant agent, wherein the polymorphic site(s) are selected from one or more of the following: rs2069912; rs7242; rs2070682; rs11178; rs2227706; and rs2227684; one or more polymorphic sites in linkage disequilibrium thereto; and a combination(s) thereof serious adverse event genotype is selected from one or more of the following:
(a) rs2069912 TT;
(b) rs7242 GG;
(c) rs2070682 CC;
(d) rs11178 CC;
(e) rs2227706 AA;
(f) rs2227684AA;
(g) one or more polymorphic sites in linkage disequilibrium therewith; and
(h) a combination(s) thereof selected from one or more of the following:
(i) rs7242 GG/rs2069912 TT;
(ii) rs2070682 CC/rs2069912 TT;
(iii) rs11178 CC/rs2069912 TT;
(iv) rs2227706 AA/rs2069912 TT;
(v) rs2227684 AA/rs2069912 TT; and
(vi) one or more polymorphic sites in linkage disequilibrium thereto selected from one or more of the polymorphic sites listed in TABLE 1B.

42.-44. (canceled)

45. The method of claim 41, further comprising obtaining polymorphism sequence information for the subject.

46. The method of claim 41, wherein the genotype is determined using a nucleic acid sample from the subject.

47. The method of claim 46, further comprising obtaining the nucleic acid sample from the subject.

48. The method of claim 41, wherein said genotype is determined using one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

49. The method of claim 41, wherein the subject is critically ill with an inflammatory condition.

50. The method of claim 49, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

51. (canceled)

52. The method of claim 41, further comprising selective non-administration of an anti-inflammatory agent or an anti-coagulant agent, wherein a subject has one or more serious adverse event genotype(s) or serious adverse event genotype combinations.

53-79. (canceled)

80. A method for selecting a group of subjects for determining the efficacy of a candidate drug known or suspected of being useful for the treatment of an inflammatory condition, the method comprising determining a genotype at one or more of the following polymorphic sites: rs7242; rs2070682; rs11178; rs2227706; and rs2227684; or one or more polymorphic sites in linkage disequilibrium thereto, wherein said genotype is indicative of the subject's response to the candidate drug and sorting subjects based on their genotype.

81. The method of claim 80 further comprising, administering the candidate drug to the subjects or a subset of subjects and determining each subject's ability to recover from the inflammatory condition.

82. The method of claim 81, further comprising comparing subject response to the candidate drug based on genotype of the subject.

83-100. (canceled)

101. Two or more oligonucleotides or peptide nucleic acids of about 10 to about 400 nucleotides that hybridize specifically to a sequence contained in a human target sequence, a complementary sequence of the target sequence or RNA equivalent of the target sequence and wherein the oligonucleotides or peptide nucleic acids are operable in determining the presence or absence of two or more improved response genotype(s) in the target sequence selected from of the following polymorphic sites: rs7242; rs2070682; rs11178; rs2227706; rs2227684 and rs2069912 or one or more polymorphic sites in linkage disequilibrium thereto. wherein the improved response genotype is selected from one or more of the following: rs7242 GT; rs7242 TT; rs2070682 CT; rs2070682 TT; rs11178 CT; rs11178 TT; rs2227706 AG; rs2227706 GG; rs2227684AG; rs2227684 GG; rs7242 GT/rs2069912 CC; rs7242 GT/rs2069912 CT; rs7242 TT/rs2069912 CT; rs7242 TT/rs2069912 CC; rs2070682 CT/rs2069912 CC; rs2070682 CT/rs2069912 CT; rs2070682 TT/rs2069912 CT; rs2070682 TT/rs2069912 CC; rs11178 CT/rs2069912 CC; rs11178 CT/rs2069912 CT; rs11178 TT/rs2069912 CT; rs11178 TT/rs2069912 CC; rs2227706 AG/rs2069912 CC; rs2227706 AG/rs2069912 CT; rs2227706 GG/rs2069912 CT; rs2227706 GG/rs2069912 CC; rs2227684 AG/rs2069912 CC; rs2227684 AG/rs2069912 CT; rs2227684 GG/rs2069912 CT; and rs2227684 GG/rs2069912 CC; or one or more polymorphic sites in linkage disequilibrium therewith selected from one or more of the polymorphic sites listed in TABLE 1B; or
two or more oligonucleotides or peptide nucleic acids selected from the group consisting of:
(a) an oligonucleotide or peptide nucleic acid that hybridizes under high stringency conditions to a nucleic acid molecule comprising SEQ ID NO:1 having a G at position 301 but not to a nucleic acid molecule comprising SEQ ID NO:1 having T at position 301;
(b) an oligonucleotide or peptide nucleic acid that hybridizes under high stringency conditions to a nucleic acid molecule comprising SEQ ID NO:1 having a T at position 301 but not to a nucleic acid molecule comprising SEQ ID NO:1 having G at position 301;
(c) an oligonucleotide or peptide nucleic acid that hybridizes under high stringency conditions to a nucleic acid molecule comprising SEQ ID NO:2 having a C at position 201 but not to a nucleic acid molecule comprising SEQ ID NO:2 having T at position 201;
(d) an oligonucleotide or peptide nucleic acid that hybridizes under high stringency conditions to a nucleic acid molecule comprising SEQ ID NO:2 having an T at position 201 but not to a nucleic acid molecule comprising SEQ ID NO:2 having C at position 201;
(e) an oligonucleotide or peptide nucleic acid that hybridizes under high stringency conditions to a nucleic acid molecule comprising SEQ ID NO:3 having a C at position 301 but not to a nucleic acid molecule comprising SEQ ID NO:3 having T at position 301;
(f) an oligonucleotide or peptide nucleic acid that hybridizes under high stringency conditions to a nucleic acid molecule comprising SEQ ID NO:3 having a T at position 301 but not to a nucleic acid molecule comprising SEQ ID NO:3 having C at position 301;
(g) an oligonucleotide or peptide nucleic acid that hybridizes under high stringency conditions to a nucleic acid molecule comprising SEQ ID NO:4 having a G at position 301 but not to a nucleic acid molecule comprising SEQ ID NO:4 having C at position 301;
(h) an oligonucleotide or peptide nucleic acid that hybridizes under high stringency conditions to a nucleic acid molecule comprising SEQ ID NO:4 having a C at position 301 but not to a nucleic acid molecule comprising SEQ ID NO:4 having a G at position 301;
(i) an oligonucleotide or peptide nucleic acid that hybridizes under high stringency conditions to a nucleic acid molecule comprising SEQ ID NO:5 having a C at position 301 but not to a nucleic acid molecule comprising SEQ ID NO:5 having a T at position 301;
(j) an oligonucleotide or peptide nucleic acid that hybridizes under high stringency conditions to a nucleic acid molecule comprising SEQ ID NO:5 having a T at position 301 but not to a nucleic acid molecule comprising SEQ ID NO:5 having a C at position 301;
(k) an oligonucleotide or peptide nucleic acid that hybridizes under high stringency conditions to a nucleic acid molecule comprising SEQ ID NO:6 having a C at position 301 but not to a nucleic acid molecule comprising SEQ ID NO:6 having a T at position 301;
(l) an oligonucleotide or peptide nucleic acid that hybridizes under high stringency conditions to a nucleic acid molecule comprising SEQ ID NO:6 having a T at position 301 but not to a nucleic acid molecule comprising SEQ ID NO:6 having a C at position 301;
(m) an oligonucleotide or peptide nucleic acid that hybridizes under high stringency conditions to a nucleic acid molecule comprising SEQ ID NO:7 having a G at position 468 but not to a nucleic acid molecule comprising SEQ ID NO:7 having an A at position 468;
(n) an oligonucleotide or peptide nucleic acid that hybridizes under high stringency conditions to a nucleic acid molecule comprising SEQ ID NO:7 having an A at position 468 but not to a nucleic acid molecule comprising SEQ ID NO:7 having a G at position 468;
(o) an oligonucleotide or peptide nucleic acid that hybridizes under high stringency conditions to a nucleic acid molecule comprising SEQ ID NO:8 having a C at position 709 but not to a nucleic acid molecule comprising SEQ ID NO:8 having a T at position 709;
(p) an oligonucleotide or peptide nucleic acid that hybridizes under high stringency conditions to a nucleic acid molecule comprising SEQ ID NO:8 having a T at position 709 but not to a nucleic acid molecule comprising SEQ ID NO:8 having a C at position 709;
(q) an oligonucleotide or peptide nucleic acid that hybridizes under high stringency conditions to a nucleic acid molecule comprising SEQ ID NO:9 having a G at position 301 but not to a nucleic acid molecule comprising SEQ ID NO:9 having an A at position 301;
(r) an oligonucleotide or peptide nucleic acid that hybridizes under high stringency conditions to a nucleic acid molecule comprising SEQ ID NO:9 having an A at position 301 but not to a nucleic acid molecule comprising SEQ ID NO:9 having a G at position 301;
(s) an oligonucleotide or peptide nucleic acid that hybridizes under high stringency conditions to a nucleic acid molecule comprising SEQ ID NO:10 having an A at position 301 but not to a nucleic acid molecule comprising SEQ ID NO:10 having a G at position 301;
(t) an oligonucleotide or peptide nucleic acid that hybridizes under high stringency conditions to a nucleic acid molecule comprising SEQ ID NO:10 having a G at position 301 but not to a nucleic acid molecule comprising SEQ ID NO:10 having an A at position 301;
(u) an oligonucleotide or peptide nucleic acid that hybridizes under high stringency conditions to a nucleic acid molecule comprising SEQ ID NO:11 having a T at position 301 but not to a nucleic acid molecule comprising SEQ ID NO:11 having a C at position 301;
(v) an oligonucleotide or peptide nucleic acid that hybridizes under high stringency conditions to a nucleic acid molecule comprising SEQ ID NO:11 having a C at position 301 but not to a nucleic acid molecule comprising SEQ ID NO:11 having a T at position 301;
(w) an oligonucleotide or peptide nucleic acid that hybridizes under high stringency conditions to a nucleic acid molecule comprising SEQ ID NO:12 having a C at position 256 but not to a nucleic acid molecule comprising SEQ ID NO:12 having a T at position 256;
(x) an oligonucleotide or peptide nucleic acid that hybridizes under high stringency conditions to a nucleic acid molecule comprising SEQ ID NO:12 having a T at position 256 but not to a nucleic acid molecule comprising SEQ ID NO:12 having a C at position 256;
(y) an oligonucleotide or peptide nucleic acid that hybridizes under high stringency conditions to a nucleic acid molecule comprising SEQ ID NO:13 having a G at position 201 but not to a nucleic acid molecule comprising SEQ ID NO:13 having an A at position 201;
(z) an oligonucleotide or peptide nucleic acid that hybridizes under high stringency conditions to a nucleic acid molecule comprising SEQ ID NO:13 having an A at position 201 but not to a nucleic acid molecule comprising SEQ ID NO:13 having a G at position 201;
(aa) an oligonucleotide or peptide nucleic acid that hybridizes under high stringency conditions to a nucleic acid molecule comprising SEQ ID NO:14 having a G at position 201 but not to a nucleic acid molecule comprising SEQ ID NO:14 having a C at position 201;
(bb) an oligonucleotide or peptide nucleic acid that hybridizes under high stringency conditions to a nucleic acid molecule comprising SEQ ID NO:14 having a C at position 201 but not to a nucleic acid molecule comprising SEQ ID NO:14 having a G at position 201;
(cc) an oligonucleotide or peptide nucleic acid that hybridizes under high stringency conditions to a nucleic acid molecule comprising SEQ ID NO:15 having a C at position 501 but not to a nucleic acid molecule comprising SEQ ID NO:15 having a T at position 501;
(dd) an oligonucleotide or peptide nucleic acid that hybridizes under high stringency conditions to a nucleic acid molecule comprising SEQ ID NO:15 having a T at position 501 but not to a nucleic acid molecule comprising SEQ ID NO:15 having a C at position 501;
(ee) an oligonucleotide or peptide nucleic acid that hybridizes under high stringency conditions to a nucleic acid molecule comprising SEQ ID NO:16 having a C at position 201 but not to a nucleic acid molecule comprising SEQ ID NO:16 having a T at position 201;
(ff) an oligonucleotide or peptide nucleic acid that hybridizes under high stringency conditions to a nucleic acid molecule comprising SEQ ID NO:16 having a T at position 201 but not to a nucleic acid molecule comprising SEQ ID NO:16 having a C at position 201;
(gg) an oligonucleotide or peptide nucleic acid that hybridizes under high stringency conditions to a nucleic acid molecule comprising SEQ ID NO:17 having a G at position 301 but not to a nucleic acid molecule comprising SEQ ID NO:17 having an A at position 301;
(hh) an oligonucleotide or peptide nucleic acid that hybridizes under high stringency conditions to a nucleic acid molecule comprising SEQ ID NO:17 having an A at position 301 but not to a nucleic acid molecule comprising SEQ ID NO:17 having a G at position 301;
(ii) an oligonucleotide or peptide nucleic acid that hybridizes under high stringency conditions to a nucleic acid molecule comprising SEQ ID NO:18 having a G at position 980 but not to a nucleic acid molecule comprising SEQ ID NO:18 having a T at position 980;
(jj) an oligonucleotide or peptide nucleic acid that hybridizes under high stringency conditions to a nucleic acid molecule comprising SEQ ID NO:18 having a T at position 980 but not to a nucleic acid molecule comprising SEQ ID NO:18 having a G at position 980;
(kk) an oligonucleotide or peptide nucleic acid that hybridizes under high stringency conditions to a nucleic acid molecule comprising SEQ ID NO:19 having a C at position 301 but not to a nucleic acid molecule comprising SEQ ID NO:19 having a G at position 301;
(ll) an oligonucleotide or peptide nucleic acid that hybridizes under high stringency conditions to a nucleic acid molecule comprising SEQ ID NO:19 having a G at position 301 but not to a nucleic acid molecule comprising SEQ ID NO:19 having a C at position 301;
(mm) an oligonucleotide or peptide nucleic acid that hybridizes under high stringency conditions to a nucleic acid molecule comprising SEQ ID NO:20 having a G at position 301 but not to a nucleic acid molecule comprising SEQ ID NO:20 having an A at position 301;
(nn) an oligonucleotide or peptide nucleic acid that hybridizes under high stringency conditions to a nucleic acid molecule comprising SEQ ID NO:20 having an A at position 301 but not to a nucleic acid molecule comprising SEQ ID NO:20 having a G at position 301;
(oo) an oligonucleotide or peptide nucleic acid that hybridizes under high stringency conditions to a nucleic acid molecule comprising SEQ ID NO:21 having an A at position 301 but not to a nucleic acid molecule comprising SEQ ID NO:21 having a G at position 301;
(pp) an oligonucleotide or peptide nucleic acid that hybridizes under high stringency conditions to a nucleic acid molecule comprising SEQ ID NO:21 having a G at position 301 but not to a nucleic acid molecule comprising SEQ ID NO:21 having an A at position 301;
(qq) an oligonucleotide or peptide nucleic acid that hybridizes under high stringency conditions to a nucleic acid molecule comprising SEQ ID NO:22 having an A at position 301 but not to a nucleic acid molecule comprising SEQ ID NO:22 having a G at position 301;
(rr) an oligonucleotide or peptide nucleic acid that hybridizes under high stringency conditions to a nucleic acid molecule comprising SEQ ID NO:22 having a G at position 301 but not to a nucleic acid molecule comprising SEQ ID NO:22 having an A at position 301;
(ss) an oligonucleotide or peptide nucleic acid that hybridizes under high stringency conditions to a nucleic acid molecule comprising SEQ ID NO:23 having a C at position 301 but not to a nucleic acid molecule comprising SEQ ID NO:23 having a T at position 301;
(tt) an oligonucleotide or peptide nucleic acid that hybridizes under high stringency conditions to a nucleic acid molecule comprising SEQ ID NO:23 having a T at position 301 but not to a nucleic acid molecule comprising SEQ ID NO:23 having a C at position 301;
(uu) an oligonucleotide or peptide nucleic acid capable of hybridizing under high stringency conditions to a nucleic acid molecule comprising a first allele for a given polymorphism selected from the polymorphisms listed in TABLE 1D but not capable of hybridizing under high stringency conditions to a nucleic acid molecule comprising a second allele for the given polymorphism selected from the polymorphisms listed in TABLE 1D; and
(vv) an oligonucleotide or peptide nucleic acid capable of hybridizing under high stringency conditions to a nucleic acid molecule comprising the second allele for a given polymorphism selected from the polymorphisms listed in TABLE 1D but not capable of hybridizing under high stringency conditions to a nucleic acid molecule comprising the first allele for the given polymorphism selected from the polymorphisms listed in TABLE 1D.

102-104. (canceled)

105. An array of oligonucleotides or peptide nucleic acids attached to a solid support, the array comprising two or more of the oligonucleotides or peptide nucleic acids set out in claim 101.

106. A composition comprising an addressable collection of two or more oligonucleotides or peptide nucleic acids, the two or more oligonucleotides or peptide nucleic acids consisting essentially of two or more nucleic acid molecules set out in SEQ ID NO:1-23 or compliments, fragments, variants, or analogs thereof.

107. The oligonucleotides or peptide nucleic acids of claim 101, further comprising one or more of the following: a detectable label; a quencher; a mobility modifier; a contiguous non-target sequence situated 5′ or 3′ to the target sequence or 5′ and 3′ to the target sequence.


SEQUENCE LISTING (23 sequences)

SEQ ID NO: 1 - DNA, 601 bp, HOMO SAPIENS
acattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaakgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttc

SEQ ID NO: 2 - DNA, 401 bp, HOMO SAPIENS
cccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgcctttycccccatcccttcttgctcacacccccaacttgatctctccctcctaactgtgccctgcacccaagacagacacttcacagagcccaggagacacctggggacccttcctgggtgataggtctgtctatcctccaggtgtccctgcccaaggggagaagcatggggaatacttggttgggggaggagaggaagactgggg

SEQ ID NO: 3 - DNA, 601 bp, HOMO SAPIENS
tccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggayggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgact

SEQ ID NO: 4 - DNA, 601 bp, HOMO SAPIENS
tttggaaggccgaggcaagtgaatcacctgagtccaggagttcgagaccagcctgggcaacatggcaaaacctcgtctccacaaaaaaaaatacaaaaatgagctgggcgtggtggcgggcgccagtagtcccagctactttggaggctgaggtgggaggattgcctgagccaaggaggtcgaggttgcagtgagccaagatcacgccactacactccagcctggggtgacagagcaagaccccggctcaaaaaaaaaaaaaaaaaagtgctgccctcacgtgccagtcatcagcagcttstgtgttgccaccagccgcggggccagcctgaacacttgcttcctgaaccaggtctttgatctcctcgccaccagaggtcgctttcgctccagtggaaagctcttccctgaagcaaagcagctgtggcttccaccaaaatcttgccaagttggaactccattcaatttaacgagtatttatttaaaagctgacctttagactgggtactgtgaagcattaaaaagaaagtgtcagctattcttaatagcagatatctaatgacaagggacatcaaaaagctggggaaaaaaagctatgctg

SEQ ID NO: 5 - DNA, 601 bp, HOMO SAPIENS
tcttcacagctgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagtaagccaccccgctatctccccgacctaccaacccctctctcctggctccctaaagtcaccgcccccaggttgaatttcccagatctgtgaygcttgcaggacatgcatgtgtgggaggctgatgggaaactgtggcctgggtttgattatgagtcttgcaatcatccctccccctgtttctgctggagggcaggggacagctcttcctgaccacacccccacattgactatccccagaatacccagcaaaagcccccaaaaggagagtcagagaaatgagggaggtgggggcccaatcagtccacatctacttagggtcgccccatcagcacttccatccccaaccctttcaagtcaacatccaaacaaaagaaatcacttccaaggacgg

SEQ ID NO: 6 - DNA, 601 bp, HOMO SAPIENS
ttgagtccagcagttcaaggctgcagtgagctatgtttgcaccaccacactccagcctgagtcacagaacaagacctcatctctaaaaaacaaacaaaaaccaaatccacatatcctaaaaaatgctccttttcagcattctcttctctatggacaaagggctggatgctttaagaaccaaatcttaggctgggcacggtggctcacgcctctaatcctagcactttgagaggccaaggcgggcagattgcctgagcacaggagttcgagaccagcctggccaacatggtgaaaccctgtytctgtcaaaaatacaaaaaattagccaggtgtgttggcgcgtgcctataatcccagctgctcgggaggatgaggttcaaagaatcacttgaacccgggaggcagaggctgcagtgagctgagatcatgccactgcactccagcctgggtgacagagcaagactttgtctccaaaaaaaggaactagacgggttcatttaaacccctgactgcagccctttgacatacatccaattgaggactggggactccgggaaacatctaaaaggcttaaaaactttgtctaacttcagccgggcat

SEQ ID NO: 7 - DNA, 968 bp, HOMO SAPIENS
ccaagcaggagacatcaggataatgggaacagaagacaggaggtttatcccatgaaggatgaagaagctgaaatccagagattccctcagggccacatttgtccacctgactccagggtctcatcttcgtgtgttgctagtgtgattacctggggatgagaaatcctgctgggggagttgaggttaagaggatgaggactccaggtgctgtggctcacgcctgtaatcccagcactttgggaggccaaggcaggtggatcaggagtttgaggtcaggagtttgagaccagcctggccaacatggtgaaaccctgtctctactaaaaatgcaaaaattagccaggtgtggtggcaggcgcctgtaatcccagctactcgggaggctgaggcaggagaatcacttgagcccgggaggtggaggttgcagtgagccgaacgaaattgagccacttcaccccagcctgggcraaagagtgaaattccattcaaaaaaaaaaaaaaaaaaaaaaggatgaggactgggatgaactggtggctgggtgtggggaaaatggaagtgaaggaaggccaaaagagacagagaaggcctggcgcggcgactcacgcctataatcccagcactttgggaggctgagaagggggattgcttgaggccagaagttgaataccagtctgggcagcatagcaagaccctgcctctacaaaaaaaaaattttttttaattagccaggcttggtgacatgcatctgtagtctactcaagaagctgaggtgaggccaggcacggtggctcacgcctgtattcccagcactttgggaggtcaaggcgggtggatgacctgaggtcaggagttcaagaccagcctggccaacatggtgaaaccccatctgtataaaaatacaaaaattagctgggcatgatagcaggtgcctgtaattccagctactcaggaggctgaggtgggagaa

SEQ ID NO: 8 - DNA, 1351 bp, HOMO SAPIENS
cagagattccctcagggccacatttgtccacctgactccagggtctcatcttcgtgtgttgctagtgtgattacctggggatgagaaatcctgctgggggagttgaggttaagaggatgaggactccaggtgctgtggctcacgcctgtaatcccagcactttgggaggccaaggcaggtggatcaggagtttgaggtcaggagtttgagaccagcctggccaacatggtgaaaccctgtctctactaaaaatgcaaaaattagccaggtgtggtggcaggcgcctgtaatcccagctactcgggaggctgaggcaggagaatcacttgagcccgggaggtggaggttgcagtgagccgaacgaaattgagccacttcaccccagcctgggcaaaagagtgaaattccattcaaaaaaaaaaaaaaaaaaaaaaggatgaggactgggatgaactggtggctgggtgtggggaaaatggaagtgaaggaaggccaaaagagacagagaaggcctggcgcggcgactcacgcctataatcccagcactttgggaggctgagaagggggattgcttgaggccagaagttgaataccagtctgggcagcatagcaagaccctgcctctacaaaaaaaaaattttttttaattagccaggcttggtgacatgcatctgtagtctactcaagaagctgaggtgaggccaggcayggtggctcacgcctgtattcccagcactttgggaggtcaaggcgggtggatgacctgaggtcaggagttcaagaccagcctggccaacatggtgaaaccccatctgtataaaaatacaaaaattagctgggcatgatagcaggtgcctgtaattccagctactcaggaggctgaggtgggagaatctattgaacccgggagggggaggttgcagtgagccgagatcatgccattgcactccagcctgggcgacagagtgagactccttctcaaaacaaacaaacaaacaaacaaacaaaatacagaagctgaggcgggaggaacatttgaaccggattcggaggctgcagtgagctatgattgcaccactgcgctccagtctgtgtgacagtgagaccctgtctcttacacacacacacacacacacacacatgcacacacacagagagagagaaattagaagatactgaattggcagaagagaagggaaatagaaattaaaatactgaataggggagcagtgaacaggggatacccaaaagccaagagcgagagagagcctggcttccagaaatagtggagaagccaggagaactaggtgaaaacccagtgctgggttgccatcagcgagagctggagcca

SEQ ID NO: 9 - DNA, 601 bp, HOMO SAPIENS
cggattcggaggctgcagtgagctatgattgcaccactgcgctccagtctgtgtgacagtgagaccctgtctcttacacacacacacacacacacacacacacatgcacacacacagagagagagaaattagaagatactgaattggcagaagagaagggaaatagaaattaaaatactgaataggggagcagtgaacaggggatacccaaaagccaagagcgagagagagcctggcttccagaaatagtggagaagccaggagaactaggtgaaaacccagtgctgggttgccatcagcragagctggagccatttccaacgaaccatcttgtcgtcttcacagctgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagtaagccaccccgctatctccccgacctaccaacccctctctcctggctccctaaag

SEQ ID NO: 10 - DNA, 601 bp, HOMO SAPIENS
caggctgaataaaattatgctgaaactactgtcttatttgaggaaagtaattagtcataggtgggagggggtggggagattgcagaagaatgttcatgaatattaggattttcagctctaaggggggactttgtaaacagctttagaagaagaaccaggccggctgggtgtggtggctcatgcctgtaatctcagcatttggggaggccaaggcgggcggatcacttgaggtcaggagtttgagaccagcctggccaacatggtgaaaccctgtctctattaaaaatacaaaaattagccrgccgtggtagcgagcgcctatgatcccagctactccggaggctgaggccagagaatcacatgaacctgggaggtggaggctgcagtgagccgagatcacgccactgcactccagcctgggggacagagcaagaatctgtttcaaaaaaaaaaaaagaaaaataggaaggaaggaaggaaaggaaaggaaagaagagagagagaaagaaagagagagaagaaagaaagaaagaaagaaagaaagaaagaaagaaagaaagaaagaaagaaagaaaaagaaaggaaagaaagaacgaacgaa

SEQ ID NO: 11 - DNA, 601 bp, HOMO SAPIENS
gctgaataaaattatgctgaaactactgtcttatttgaggaaagtaattagtcataggtgggagggggtggggagattgcagaagaatgttcatgaatattaggattttcagctctaaggggggactttgtaaacagctttagaagaagaaccaggccggctgggtgtggtggctcatgcctgtaatctcagcatttggggaggccaaggcgggcggatcacttgaggtcaggagtttgagaccagcctggccaacatggtgaaaccctgtctctattaaaaatacaaaaattagccagcygtggtagcgagcgcctatgatcccagctactccggaggctgaggccagagaatcacatgaacctgggaggtggaggctgcagtgagccgagatcacgccactgcactccagcctgggggacagagcaagaatctgtttcaaaaaaaaaaaaagaaaaataggaaggaaggaaggaaaggaaaggaaagaagagagagagaaagaaagagagagaagaaagaaagaaagaaagaaagaaagaaagaaagaaagaaagaaagaaagaaagaaaaagaaaggaaagaaagaacgaacgaacca

SEQ ID NO: 12 - DNA, 511 bp, HOMO SAPIENS
aaaaatgtttcaaaaaaataataaaataaataaatacgaagaatatgtcaggacagtcactgccttcaccttctccatttcacaccggtggtacaagaaatcagaagcctaggccaggtgtggtggttcatgcctgtaatcccagcactttgggaagccgaggtgggtggatcacctaaggtcaggagtttgagaccagcctggacaacatggtgaaaccccgtctctactaaaaatacaaaaattagccgggcgyggtggctggcgcctgtaatcccagctactcgggaggctgaggcaggagaatcacttgaagccaggaggcagaggttgcagtgagctgagattgcaccactgaactccaggctgggtggcagagcgagactccctctcaaaaaacaacaactacaaagacaacaacaaacccagaatcaaaatcctgttggtccatagacctcatgggtggaagagaccttcctacatccaggttggcccaacatgggggagtcca

SEQ ID NO: 13 - DNA, 656 bp, HOMO SAPIENS
ataaactttccacttatgcagatgggcctgctcgtaagtcactgtcactgtgggttcccaactctgttcatgacacttccttccagcaccaaatgcttcccacccctctactcccactccccattcttcaaacccagctcaagttccagttcctccacctaggacttcccatggatccagccaatatcactctcaggtccrgcgcagtggcccacgcctgtaatctcagcactttgggaggccggggcaggaagattgcttgaggccaggagtttcagaccagcctggacaacatagtgagactcttcctctaagaaaagaaagagagaaagagagagagaaagacagaggaaagagagagaaaaataaagaaagagagaaagaaagaaagaaaaagaaaaagaaagaaagaacgaaagaaagaaagaaagaaagaaagaaagaaagaaagaaagaaagaaagaaagaaagaaagaaagaaagaaagaaaaagaaaggaaggaaggaaggaaaggagggagggagggagggaaggaaggaaggaaggaaggaaggggatcaaaacatcatcactctcaactcgacactgactgagttttcttctccctggtctgtaacagtgcttggattctcgagtgttccttagctttgtgtgt

SEQ ID NO: 14 - DNA, 1464 bp, HOMO SAPIENS
aacaagatctgcctccagctaccgttattgtacatttcttgtaaacctcaaacagtggaattatttatctgctcatctgtgggcaaagagtatgaatgttgtaaatcccttaatggtaaaaatattcagtcaaaagcaaatttgtcaaggagacaccatctttttttgaaatcatttttttaggtggtttctcaagtaagstttgttgaaataatagtagcaagcggctgggcgtggtggctcaggcctgtaatcccagcactttaggaggccaaggtgggcggatcacctgaggtcaggagtttgagaccagcctggccaacaaggtgaaaccctgtctctactaaaaatacaaaaattaggccgggtgtggtggctcacacctgtaatcccagcactttgggaggccaaggcgggtggatcacgaggtcaggagttcaagaccagcctggccaggttggtgaaaccccgtctccactaaaaacacacaaaaaattaacgaggcgcagtggcgggtgcctgtagtcccagctactcaggaggctgaggcaggagaatggcgtgaaaccaggaggcagagcttgcaacgagccgagatcacgccactggactccagcctgggcaacagagtgagactccgtctcaaaaaaaaaaaaaaaaaaaaaagtagcaagcactcaggaaaactgagacacagagaggttgagtaacttgatctcattattatcattattttagagagggggtctctgttgtttaggctggagtgcagtggtgcaatcacagctcactgcagccttgaactgctgggctcaaacaatcctgtctcagcctcccaagcagctgggactacaggtgcgcaccactcactacccagctaattcgttttctttcttttttttttttttttaaaaaaaagtctcgctctgtcacccatgctggaatgcaatggcttgatctctctgctcactacaacctccgcctcccgggttcaagagattctcctttctcagcctcctgagtagctgggattacaggcatgtgccaccatgcccggttaatttttgtatttttagtagagatagggttttgccatgttggccaggctggtctcgagcttctgacctcaggtaatccacctgcctcagcctcccaaagtgctgagattacaggtgtgagccactgcacctggccccagctaattttttaattgtattttttgtagacacagggtctcactatgttgtccaagctggtctcgaactcctgggctcagcctcccaaagtgctgggattacaggcgtgagccactgcacccggccccagctaattttttaattttattttttgtagagacagggtctcactatgttgttcaagctggtcttgaactctggggctcagcctcccaaagtgctgggattacaagcatgagctactgtgcccagctgctgctatcatcat

SEQ ID NO: 15 - DNA, 701 bp, HOMO SAPIENS
agttccagttcctccacctaggacttcccatggatccagccaatatcactctcaggtccggcgcagtggcccacgcctgtaatctcagcactttgggaggccggggcaggaagattgcttgaggccaggagtttcagaccagcctggacaacatagtgagactcttcctctaagaaaagaaagagagaaagagagagagaaagacagaggaaagagagagaaaaataaagaaagagagaaagaaagaaagaaaaagaaaaagaaagaaagaacgaaagaaagaaagaaagaaagaaagaaagaaagaaagaaagaaagaaagaaagaaagaaagaaagaaagaaagaaaaagaaaggaaggaaggaaggaaaggagggagggagggagggaaggaaggaaggaaggaaggaaggggatcaaaacatcatcactctcaactcgacactgactgagttttcttctccctggtctgtaacagtgcttggattctcgagtgttcyttagctttgtgtgtgtgtgtcttaccctccccaagcccatcaaggtatcaggtttcttgaaaacaaggctctcttgttatatatactctagcatcttcttgaaaatggcttcctgaaaagtcttttattatctttctccagcatctcgaaggcttttggcagagggcacacttccctccattagtttctgttcaaatatt

SEQ ID NO: 16 - DNA, 1180 bp, HOMO SAPIENS
cgtgcagcgtcctcctccatgtagcctggctgcgtttttctctgacgttgtccggcgtgcatcgcatttccctctttacccccttgcttccttgaggagagaacagaatcccgattctgccttcttctatattttcctttttatgcattttaatcaaatttatatatgtatgaaactttaaaaatcagagttttacaactyttacatttcagcatgctgttccttggcatgggtccttttttcattcattttcattaaaaggtggacccttttaatgtggaaattcctatcttctgcctctagggacatttatcacttatttcttctacaatctcccctttacttcctctattttctctttctggacctcccattattcagacctctttcctctagttttattgtctcttctatttcccatctctttgactttgtgttttctttcagggaactttcttttttttctttttttttgagatggagtttcactcttgttgtcccaggctggagtgcaatgacgtgatctcagctcaccacaacctccgcctcctggattcaagcgattctcctgccgcagcctcccgagtagctgggattacaggcatgcgccaccacgcccagctaattttgtgtttttagtagagaaggggtttctccgtgttggtcaagctggtcttgaactcctgacctcaggtgatccacctgccttggcctcctaaagtgctgggattacaggcgtgagccaccgcgcccagcctctttcagggaactttctacaactttataattcaattcttctgcagaaaaaaatttttggccaggctcagtagctcagaccaataattccagcactttgagaggctgaggtgggaggattgcttgagcttgggagtttgagactagcctgggcaacacagtgagaccctgtctctatttttaaaaaaagtaaaaaaagatctaaaaatttaactttttattttgaaataattagatatttccaggaagctgcaaagaaatgcctggtgggcctgttggcctgtgggtttcctgcaaggccttgggaaggccctgtcattggcagaaccccagatcgtgagggctttccttttaggctgctttctaagaggactcctccaagctcttggaggatggaagacgctcacccatggtgttcggcccc

SEQ ID NO: 17 - DNA, 601 bp, HOMO SAPIENS
agcaggccttggctggcctctctgatggagcaggcatcaggcacaggccgtgggtctcaacgtgggctgggtggtcctggaccagcagcagccgccgcagcagcaaccctggtacctggttaggaacgcagaccctctgcccccatcctcccaactctgaaaaacactggcttagggaaaggcgcgatgctcaggggtcccccaaagcccgcaggcagagggagtgatgggactggaaggaggccgagtgacttggtgagggattcgggtcccttgcatgccagaggctgctgtgggagcrgacagtcgcgagagcagcactgcagctgcatggggagagggtgttgctccagggacgtgggatggaggctgggcgcgggcgggtggcgctggagggcgggggaggggcagggagcaccagctcctagcagccaacgaccatcgggcgtcgatccctgtttgtctggaagccctcccctcccctgcccgctcacccgctgccctgccccacccgggcgcgccccctccgcacaccggctgcaggagcctgacgctgcccgctctctccgcagctggccttctggtccaagcacgtcggtga

SEQ ID NO: 18 - DNA, 1180 bp, HOMO SAPIENS
cccccttgcttccttgaggagagaacagaatcccgattctgccttcttctatattttcctttttatgcattttaatcaaatttatatatgtatgaaactttaaaaatcagagttttacaacttttacatttcagcatgctgttccttggcatgggtccttttttcattcattttcattaaaaggtggacccttttaatgtggaaattcctatcttctgcctctagggacatttatcacttatttcttctacaatctcccctttacttcctctattttctctttctggacctcccattattcagacctctttcctctagttttattgtctcttctatttcccatctctttgactttgtgttttctttcagggaactttcttttttttctttttttttgagatggagtttcactcttgttgtcccaggctggagtgcaatgacgtgatctcagctcaccacaacctccgcctcctggattcaagcgattctcctgccgcagcctcccgagtagctgggattacaggcatgcgccaccacgcccagctaattttgtgtttttagtagagaaggggtttctccgtgttggtcaagctggtcttgaactcctgacctcaggtgatccacctgccttggcctcctaaagtgctgggattacaggcgtgagccaccgcgcccagcctctttcagggaactttctacaactttataattcaattcttctgcagaaaaaaatttttggccaggctcagtagctcagaccaataattccagcactttgagaggctgaggtgggaggattgcttgagcttgggagtttgagactagcctgggcaacacagtgagaccctgtctctatttttaaaaaaagtaaaaaaagatctaaaaatttaactttttattttgaaataattagatatttccaggaagctgcaaagaaatgcctggtgggcctgttggcctgtgggtttcctgcaaggccktgggaaggccctgtcattggcagaaccccagatcgtgagggctttccttttaggctgctttctaagaggactcctccaagctcttggaggatggaagacgctcacccatggtgttcggcccctcagagcagggtggggcaggggagctggtgcctgtgcaggctgtggacatttgcatgactccctgtggtcagctaaga

SEQ ID NO: 19 - DNA, 601 bp, HOMO SAPIENS
tgcacactggcctcacggctgccctgccccaacccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagasagacacttcacagagcccaggagacacctggggacccttcctgggtgataggtctgtctatcctccaggtgtccctgcccaaggggagaagcatggggaatacttggttgggggaggagaggaagactggggggatgtgtcaagatggggctgcacgtggtgtactggcagaagagtgagaggatttaacttggcagcctttacagcagcagccagggcttgagtacttatctctgggccagggactgtattggatgttttacatgacggtctcatccccatgtttttggatgagtaaat

SEQ ID NO: 20 - DNA, 601 bp, HOMO SAPIENS
cacggctgccctgccccaacccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagacagacacttcacaragcccaggagacacctggggacccttcctgggtgataggtctgtctatcctccaggtgtccctgcccaaggggagaagcatggggaatacttggttgggggaggagaggaagactggggggatgtgtcaagatggggctgcacgtggtgtactggcagaagagtgagaggatttaacttggcagcctttacagcagcagccagggcttgagtacttatctctgggccagggactgtattggatgttttacatgacggtctcatccccatgtttttggatgagtaaattgaaccttagaaa

SEQ ID NO: 21 - DNA, 601 bp, HOMO SAPIENS
gtaaggccaccatgggtccagaggatgaggctcaggggcgagctggtaaccagcaggggcctcgaggagcaggtggggactcaatgctgaggccctcttaggagttgtgggggtggctgagtggagcgattaggatgctggccctatgatgtcggccaggcacatgtgactgcaagaaacagaattcaggaagaagctccaggaaagagtgtggggtgaccctaggtggggactcccaccagccacagtgtaggtggttcagtccaccctccagccactgctgagcaccactgcctccccrtcccacctcacaaagaggggacctaaagaccaccctgcttccacccatgcctctgctgatcagggtgtgtgtgtgaccgaaactcacttctgtccacataaaatcgctcactctgtgcctcacatcaaagggagaaaatctgattgttcagggggtcggaagacagggtctgtgtcctatttgtctaagggtcagagtcctttggagcccccagagtcctgtggacgtggccctaggtagtagggtgagcttggtaacggggctggcttcctgagacaaggctcagacccgctctgtccc

SEQ ID NO: 22 - DNA, 601 bp, HOMO SAPIENS
cagagtcctgtggacgtggccctaggtagtagggtgagcttggtaacggggctggcttcctgagacaaggctcagacccgctctgtccctggggatcgcttcagccactaggacctgaaaattgtgcacggcctgggcccccttccaaggcatccagggatgctttccagtggaggctttcagggcaggagaccctctggcctgcaccctctcttgccctcagcctccacctccttgactggacccccatctggacctccatccccaccacctctttccccagtggcctccctggcagacrccacagtgactttctgcaggcacatatctgatcacatcaagtccccaccgtgctcccacctcacccatggtctctcagccccagcaggccttggctggcctctctgatggagcaggcatcaggcacaggccgtgggtctcaacgtgggctgggtggtcctggaccagcagcagccgccgcagcagcaaccctggtacctggttaggaacgcagaccctctgcccccatcctcccaactctgaaaaacactggcttagggaaaggcgcgatgctcaggggtcccccaaagcccgcaggcag

SEQ ID NO: 23 - DNA, 601 bp, HOMO SAPIENS
cccccgtggggcttggcttagaattcccaggtgctcttcccagggaaccatcagtctggactgagaggaccttctctctcaggtgggacccggccctgtcctccctggcagtgccgtgttctgggggtcctcctctctgggtctcactgcccctggggtctctccagctacctttgctccacgttcctttgtggctctggtctgtgtctggggtttccaggggtctcgggcttccctgctgcccattccttctctggtctcacggctccgtgactcctgaaaaccaaccagcatcctaccyctttgggattgacacctgttggccactccttctggcaggaaaagtcaccgttgatagggttccacggcatagacaggtggctccgcgccagtgcctgggacgtgtgggtgcacagtctccgggtgaaccttcttcaggccctctgcccaggcctgcaggggcacagcagtgggtgggcctcaggaaagtgccactggggagaggctccccgcagcccactctgactgtgccctctgccctgcaggagagtatgacctgcggcgctgggagaagtgggagctggacctggacatcaaggag